SEQUENCE LISTING (81 sequences)

SEQ ID NO: 1 - DNA, 21 bp, Artificial Sequence
  302F-universal-Tm54
cacactggractgagayacgg

SEQ ID NO: 2 - DNA, 19 bp, Artificial Sequence
  333F-universal-Tm55-2X
actcctacgggaggcwgca

SEQ ID NO: 3 - DNA, 18 bp, Artificial Sequence
  528F-universal-Tm57-4X
gtgccagcagyygcggta

SEQ ID NO: 4 - DNA, 21 bp, Artificial Sequence
  802R-universal-Tm52-8X
gaytacyrgggtatctaatcc

SEQ ID NO: 5 - DNA, 22 bp, Artificial Sequence
  897R-universal-Tm53-6X
ccccgtcaatthmtttgagttt

SEQ ID NO: 6 - DNA, 22 bp, Artificial Sequence
  1072R-universal-Tm55-2X
cgttrcgggacttaacccaaca

SEQ ID NO: 7 - DNA, 18 bp, Artificial Sequence
  1166R-Tm53-4X
tcrtccycaccttcctcc

SEQ ID NO: 8 - DNA, 19 bp, Artificial Sequence
  1380R-Tm52-8X
yccgrgaacgtattcacsg

SEQ ID NO: 9 - DNA, 17 bp, Artificial Sequence
  18S-304F-2-fold-Tm50.7
ggtattggcctaccgrg

SEQ ID NO: 10 - DNA, 19 bp, Artificial Sequence
  18S-413F-0-fold-Tm51.1
tacccaatcctgacacagg

SEQ ID NO: 11 - DNA, 21 bp, Artificial Sequence
  18S-877R-2-fold-Tm53.4
gctttcgcagtrgttcgtctt

SEQ ID NO: 12 - DNA, 20 bp, Artificial Sequence
  18S-931R-0-fold-Tm51.8
cgtcttcgatcccctaactt

SEQ ID NO: 13 - DNA, 19 bp, Artificial Sequence
  18S-1018F-0-fold-Tm51.1
gactccttcagcaccttga

SEQ ID NO: 14 - DNA, 21 bp, Artificial Sequence
  18S-1619R-0-fold-Tm50.5
cgaataattcaccggatcact

SEQ ID NO: 15 - DNA, 26 bp, Artificial Sequence
  18S-1679R-0-fold-Tm51.7
agttttgtgaaccttatcacttaaag

SEQ ID NO: 16 - DNA, 17 bp, Artificial Sequence
  Mycobacterium-rpoB-259F-4XTm47.1-51.9
cacggcaayaagggygt

SEQ ID NO: 17 - DNA, 18 bp, Artificial Sequence
  Mycobacterium-rpoB-274F-6XTm45.8-50.3
gtbatcggcaagatyctc

SEQ ID NO: 18 - DNA, 17 bp, Artificial Sequence
  Mycobacterium-rpoB-697R-1XTm51.9
tcaccgggtacgggaac

SEQ ID NO: 19 - DNA, 17 bp, Artificial Sequence
  Mycobacterium-rpoB-755R-4XTm47.1-49.5
gcgtgratcttgtcrtc

SEQ ID NO: 20 - DNA, 18 bp, Artificial Sequence
  Mycobacterium-hsp65-322F-2XTm=52.6
tacgagaagatcggcgcy

SEQ ID NO: 21 - DNA, 18 bp, Artificial Sequence
  Mycobacterium-hsp65-282F-1XTm=52.6
ggtgtgtccatcgccaag

SEQ ID NO: 22 - DNA, 18 bp, Artificial Sequence
  Mycobacterium-hsp65-650R-3XTm=51.9
ctcgttgccvaccttgtc

SEQ ID NO: 23 - DNA, 18 bp, Artificial Sequence
  Mycobacterium-hsp65-670R-2XTm=52.6
ctcgacggtgatgacrcc

SEQ ID NO: 24 - DNA, 16 bp, Artificial Sequence
  Fungal-18S-SSU-forward1-4X
gtacacackccygtcg

SEQ ID NO: 25 - DNA, 23 bp, Artificial Sequence
  Fungal-18S-SSU-forward2-6X
tgyaattdttgctcttcaacgag

SEQ ID NO: 26 - DNA, 20 bp, Artificial Sequence
  Fungal-296R-4X
gctscgttcttcatcgatsc

SEQ ID NO: 27 - DNA, 21 bp, Artificial Sequence
  Fungal-350R-4X
gttcaagaytcratgattcac

SEQ ID NO: 28 - DNA, 20 bp, Artificial Sequence
  Fungal-296R-Pneumocystis
gccacgttcttcatcgacgc

SEQ ID NO: 29 - DNA, 22 bp, Artificial Sequence
  Fungal-350R-Pneumocystis
gttcaaaaattcgatgattcac

SEQ ID NO: 30 - protein, 7 aa, Artificial Sequence
  Nuclear Localization Sequence
PKKKRKV

SEQ ID NO: 31 - protein, 16 aa, Artificial Sequence
  Nuclear Localization Sequence
KRPAATKKAGQAKKKK

SEQ ID NO: 32 - protein, 9 aa, Artificial Sequence
  Nuclear Localization Sequence
PAAKRVKLD

SEQ ID NO: 33 - protein, 11 aa, Artificial Sequence
  Nuclearl Localization Sequence
RQRRNELKRSP

SEQ ID NO: 34 - protein, 38 aa, Artificial Sequence
  Nuclear Localization Sequence
NQSSNFGPMKGGNFGGRSSGPYGGGGQYFAKPRNQGGY

SEQ ID NO: 35 - protein, 42 aa, Artificial Sequence
  Nuclear Localization Sequence
RMRIZFKNKGKDTAELRRRRVEVSVELRKAKKDEQILKRRNV

SEQ ID NO: 36 - protein, 8 aa, Artificial Sequence
  Nuclear Localization Sequence
VSRKRPRP

SEQ ID NO: 37 - protein, 8 aa, Artificial Sequence
  Nuclear Localization Sequence
PPKKARED

SEQ ID NO: 38 - protein, 8 aa, Artificial Sequence
  Nuclear Localization Sequence
PQPKKKPL

SEQ ID NO: 39 - protein, 12 aa, Artificial Sequence
  Nuclear Localization Sequence
SALIKKKKKMAP

SEQ ID NO: 40 - protein, 5 aa, Artificial Sequence
  Nuclear Localization Sequence
DRLRR

SEQ ID NO: 41 - protein, 7 aa, Artificial Sequence
  Nuclear Localization Sequence
PKQKKRK

SEQ ID NO: 42 - protein, 10 aa, Artificial Sequence
  Nuclear Localization Sequence
RKLKKKIKKL

SEQ ID NO: 43 - protein, 10 aa, Artificial Sequence
  Nuclear Localization Sequence
REKKKFLKRR

SEQ ID NO: 44 - protein, 20 aa, Artificial Sequence
  Nuclear Localization Sequence
KRKGDEVDGVDEVAKKKSKK

SEQ ID NO: 45 - protein, 17 aa, Artificial Sequence
  Nuclear Localization Sequence
RKCLQAGMNLEARKTKK

SEQ ID NO: 46 - DNA, 60 bp, Artificial Sequence
  Conserved region of 16S rRNA
gacgggggcccgcacaagcggtggagcatgtggtttaattcgaagcaacgcgaagaacct

SEQ ID NO: 47 - DNA, 60 bp, Artificial Sequence
  Conserved Region of 16S rRNA
gacgggggcccgcacaagcggtggagcatgtggtttaattcgatgcaacgcgaagaacct

SEQ ID NO: 48 - DNA, 60 bp, Artificial Sequence
  Conserved Region of 16S rRNA
gacggggacccgcacaagcggtggagcatgtggtttaattcgaagcaacgcgaagaacct

SEQ ID NO: 49 - DNA, 60 bp, Artificial Sequence
  Conserved Region of 16S rRNA
gacgggggcccgcacaagcggtggagcatgtggtttaattcgatgatacgcgaggaacct

SEQ ID NO: 50 - DNA, 60 bp, Artificial Sequence; identical to SEQ ID NO: 47
  Conserved Region of 16S rRNA

SEQ ID NO: 51 - DNA, 20 bp, Artificial Sequence
  Conserved Guide 84
attcgatgatacgcgaggaa

SEQ ID NO: 52 - protein, 1129 aa, Alicyclobacillus acidoterrestris
MAVKSIKVKLRLDDMPEIRAGLWKLHKEVNAGVRYYTEWLSLLRQENLYRRSPNGDGEQECDKTAEECKAELLERLRARQVENGHRGPAGSDDELLQLARQLYELLVPQAIGAKGDAQQIARKFLSPLADKDAVGGLGIAKAGNKPRWVRMREAGEPGWEEEKEKAETRKSADRTADVLRALADFGLKPLMRVYTDSEMSSVEWKPLRKGQAVRTWDRDMFQQAIERMMSWESWNQRVGQEYAKLVEQKNRFEQKNFVGQEHLVHLVNQLQQDMKEASPGLESKEQTAHYVTGRALRGSDKVFEKWGKLAPDAPFDLYDAEIKNVQRRNTRRFGSHDLFAKLAEPEYQALWREDASFLTRYAVYNSILRKLNHAKMFATFTLPDATAHPIWTRFDKLGGNLHQYTFLFNEFGERRHAIRFHKLLKVENGVAREVDDVTVPISMSEQLDNLLPRDPNEPIALYFRDYGAEQHFTGEFGGAKIQCRRDQLAHMHRRRGARDVYLNVSVRVQSQSEARGERRPPYAAVFRLVGDNHRAFVHFDKLSDYLAEHPDDGKLGSEGLLSGLRVMSVDLGLRTSASISVFRVARKDELKPNSKGRVPFFFPIKGNDNLVAVHERSQLLKLPGETESKDLRAIREERQRTLRQLRTQLAYLRLLVRCGSEDVGRRERSWAKLIEQPVDAANHMTPDWREAFENELQKLKSLHGICSDKEWMDAVYESVRRVWRHMGKQVRDWRKDVRSGERPKIRGYAKDVVGGNSIEQIEYLERQYKFLKSWSFFGKVSGQVIRAEKGSRFAITLREHIDHAKEDRLKKLADRIIMEALGYVYALDERGKGKWVAKYPPCQLILLEELSEYQFNNDRPPSENNQLMQWSHRGVFQELINQAQVHDLLVGTMYAAFSSRFDARTGAPGIRCRRVPARCTQEHNPEPFPWWLNKFVVEHTLDACPLRADDLIPTGEGEIFVSPFSAEEGDFHQIHADLNAAQNLQQRLWSDFDISQIRLRCDWGEVDGELVLIPRLTGKRTADSYSNKVFYTNTGVTYYERERGKKRRKVFAQEKLSEEEAELLVEADEAREKSVVLMRDPSGIINRGNWTRQKEFWSMVNQRIEGYLVKQIRSRVPLQDSACENTGDI

SEQ ID NO: 53 - protein, 872 aa, Alicyclobacillus contaminans
MGFNTAELLRKVEEEMRKTSVGFDTDNPFAHRITRRAIRGWDRIAEAWRRLPPDAPESEYIEAFKDIQRKNPRKIGSEPLFKNLAAPGVRSELLNNPQVLITFAKYNELQRQLAKAKQFAQKTLPHPVFHPVWVRYDKLGGNLHHYQIEPAVHANDTHKVKFSSLLLPQEDGSYAEVKDVTVSLAPSLQFPTGLVHPKVTTPPRTGLVTVMDEEAGKPVVCYRDRGHDALVPVAFGGAKLQFNRAHLSAGYRKGVLSAGGGGSIYFNVTLDVQVPNERDVSKTFSFSRDRDLVSLKAEELKRYMETKPLGMPGVRVMSVDLGVRYGAAISVFEVKPFAEVRKDKLHYPITGCEGFVAEHERSVILKLPGEGVRTAGKQSERKQALAAIRAEMSILRKWLRVSQVTEEDRAKAVRGLLEDERGGGWTMDPGEDSDHQPLQQFLHEARLAVGELVNLVHLSPAEWERAVIERHRRLERITASHIRVFQTMRKVWGKRRNEDAAHTGGISLAHIEHLIQQRKLFIRWSTHARTYGEVRRLPKHEGFAKRLQKHTNHVKEDRIKKLADMIVMAARGYRFLDKRARWVKTRHAPCDLILFEDLSRYRFTMDRPPTENSQLMNWSHRELLKTVKMQAALFGIGVGTVPAAFTSRFDAQTGAPGLRCKRVTKQDKEKTPFWLIQFAEITGVNVTNVEPGQLIPVDGGEWFVSPKGPRAADGLKCVHADINAAHNLQRRFWIPRLPSVKCRRYVEAEGFAAVPSSTAFMKVHGKGAFVSVDGEFYEYQKGRRVAVNRADRTSSTLDEDEGDIGEEMLVSSNGAGEFVRMFYDESGYVGYGRWMDSKVFWGKVRQIVHRAIQDQVEKRAAARGENGATSSR

SEQ ID NO: 54 - protein, 1149 aa, Desulfovibrio inopinatus
MPTRTINLKLVLGKNPENATLRRALFSTHRLVNQATKRIEEFLLLCRGEAYRTVDNEGKEAEIPRHAVQEEALAFAKAAQRHNGCISTYEDQEILDVLRQLYERLVPSVNENNEAGDAQAANAWVSPLMSAESEGGLSVYDKVLDPPPVWMKLKEEKAPGWEAASQIWIQSDEGQSLLNKPGSPPRWIRKLRSGQPWQDDFVSDQKKKQDELTKGNAPLIKQLKEMGLLPLVNPFFRHLLDPEGKGVSPWDRLAVRAAVAHFISWESWNHRTRAEYNSLKLRRDEFEAASDEFKDDFTLLRQYEAKRHSTLKSIALADDSNPYRIGVRSLRAWNRVREEWIDKGATEEQRVTILSKLQTQLRGKFGDPDLFNWLAQDRHVHLWSPRDSVTPLVRINAVDKVLRRRKPYALMTFAHPRFHPRWILYEAPGGSNLRQYALDCTENALHITLPLLVDDAHGTWIEKKIRVPLAPSGQIQDLTLEKLEKKKNRLYYRSGFQQFAGLAGGAEVLFHRPYMEHDERSEESLLERPGAVWFKLTLDVATQAPPNWLDGKGRVRTPPEVHHFKTALSNKSKHTRTLQPGLRVLSVDLGMRTFASCSVFELIEGKPETGRAFPVADERSMDSPNKLWAKHERSFKLTLPGETPSRKEEEERSIARAEIYALKRDIQRLKSLLRLGEEDNDNRRDALLEQFFKGWGEEDVVPGQAFPRSLFQGLGAAPFRSTPELWRQHCQTYYDKAEACLAKHISDWRKRTRPRPTSREMWYKTRSYHGGKSIWMLEYLDAVRKLLLSWSLRGRTYGAINRQDTARFGSLASRLLHHINSLKEDRIKTGADSIVQAARGYIPLPHGKGWEQRYEPCQLILFEDLARYRFRVDRPRRENSQLMQWNHRAIVAETTMQAELYGQIVENTAAGFSSRFHAATGAPGVRCRFLLERDFDNDLPKPYLLRELSWMLGNTKVESEEEKLRLLSEKIRPGSLVPWDGGEQFATLHPKRQTLCVIHADMNAAQNLQRRFFGRCGEAFRLVCQPHGDDVLRLASTPGARLLGALQQLENGQGAFELVRDMGSTSQMNRFVMKSLGKKKIKPLQDNNGDDELEDVLSVLPEEDDTGRITVFRDSSGIFFPCNVWIPAKQFWPAVRAMIWKVMASHSLG

SEQ ID NO: 55 - protein, 1194 aa, Desulfonatronum thiodismutans
MVLGRKDDTAELRRALWTTHEHVNLAVAEVERVLLRCRGRSYWTLDRRGDPVHVPESQVAEDALAMAREAQRRNGWPVVGEDEEILLALRYLYEQIVPSCLLDDLGKPLKGDAQKIGTNYAGPLFDSDTCRRDEGKDVACCGPFHEVAGKYLGALPEWATPISKQEFDGKDASHLRFKATGGDDAFFRVSIEKANAWYEDPANQDALKNKAYNKDDWKKEKDKGISSWAVKYIQKQLQLGQDPRTEVRRKLWLELGLLPLFIPVFDKTMVGNLWNRLAVRLALAHLLSWESWNHRAVQDQALARAKRDELAALFLGMEDGFAGLREYELRRNESIKQHAFEPVDRPYVVSGRALRSWTRVREEWLRHGDTQESRKNICNRLQDRLRGKFGDPDVFHWLAEDGQEALWKERDCVTSFSLLNDADGLLEKRKGYALMTFADARLHPRWAMYEAPGGSNLRTYQIRKTENGLWADVVLLSPRNESAAVEEKTFNVRLAPSGQLSNVSFDQIQKGSKMVGRCRYQSANQQFEGLLGGAEILFDRKRIANEQHGATDLASKPGHVWFKLTLDVRPQAPQGWLDGKGRPALPPEAKHFKTALSNKSKFADQVRPGLRVLSVDLGVRSFAACSVFELVRGGPDQGTYFPAADGRTVDDPEKLWAKHERSFKITLPGENPSRKEEIARRAAMEELRSLNGDIRRLKAILRLSVLQEDDPRTEHLRLFMEAIVDDPAKSALNAELFKGFGDDRFRSTPDLWKQHCHFFHDKAEKVVAERFSRWRTETRPKSSSWQDWRERRGYAGGKSYWAVTYLEAVRGLILRWNMRGRTYGEVNRQDKKQFGTVASALLHHINQLKEDRIKTGADMIIQAARGFVPRKNGAGWVQVHEPCRLILFEDLARYRFRTDRSRRENSRLMRWSHREIVNEVGMQGELYGLHVDTTEAGFSSRYLASSGAPGVRCRHLVEEDFHDGLPGMHLVGELDWLLPKDKDRTANEARRLLGGMVRPGMLVPWDGGELFATLNAASQLHVIHADINAAQNLQRRFWGRCGEAIRIVCNQLSVDGSTRYEMAKAPKARLLGALQQLKNGDAPFHLTSIPNSQKPENSYVMTPTNAGKKYRAGPGEKSSGEEDELALDIVEQAEELAQGRKTFFRDPSGVFFAPDRWLPSEIYWSRIRRRIWQVTLERNSSGRQERAEMDEMPY

SEQ ID NO: 56 - protein, 1388 aa, Opitutaceae bacterium TAV5
MSLNRIYQGRVAAVETGTALAKGNVEWMPAAGGDEVLWQHHELFQAAINYYLVALLALADKNNPVLGPLISQMDNPQSPYHVWGSFRRQGRQRTGLSQAVAPYITPGNNAPTLDEVFRSILAGNPTDRATLDAALMQLLKACDGAGAIQQEGRSYWPKFCDPDSTANFAGDPAMLRREQHRLLLPQVLHDPAITHDSPALGSFDTYSIATPDTRTPQLTGPKARARLEQAITLWRVRLPESAADFDRLASSLKKIPDDDSRLNLQGYVGSSAKGEVQARLFALLLFRHLERSSFTLGLLRSATPPPKNAETPPPAGVPLPAASAADPVRIARGKRSFVFRAFTSLPCWHGGDNIHPTWKSFDIAAFKYALTVINQIEEKTKERQKECAELETDFDYMHGRLAKIPVKYTTGEAEPPPILANDLRIPLLRELLQNIKVDTALTDGEAVSYGLQRRTIRGFRELRRIWRGHAPAGTVFSSELKEKLAGELRQFQTDNSTTIGSVQLFNELIQNPKYWPIWQAPDVETARQWADAGFADDPLAALVQEAELQEDIDALKAPVKLTPADPEYSRRQYDFNAVSKFGAGSRSANRHEPGQTERGHNTFTTEIAARNAADGNRWRATHVRIHYSAPRLLRDGLRRPDTDGNEALEAVPWLQPMMEALAPLPTLPQDLTGMPVFLMPDVTLSGERRILLNLPVTLEPAALVEQLGNAGRWQNQFFGSREDPFALRWPADGAVKTAKGKTHIPWHQDRDHFTVLGVDLGTRDAGALALLNVTAQKPAKPVHRIIGEADGRTWYASLADARMIRLPGEDARLFVRGKLVQEPYGERGRNASLLEWEDARNIILRLGQNPDELLGADPRRHSYPEINDKLLVALRRAQARLARLQNRSWRLRDLAESDKALDEIHAERAGEKPSPLPPLARDDAIKSTDEALLSQRDIIRRSFVQIANLILPLRGRRWEWRPHVEVPDCHILAQSDPGTDDTKRLVAGQRGISHERIEQIEELRRRCQSLNRALRHKPGERPVLGRPAKGEEIADPCPALLEKINRLRDQRVDQTAHAILAAALGVRLRAPSKDRAERRHRDIHGEYERFRAPADFVVIENLSRYLSSQDRARSENTRLMQWCHRQIVQKLRQLCETYGIPVLAVPAAYSSRFSSRDGSAGFRAVHLTPDHRHRMPWSRILARLKAHEEDGKRLEKTVLDEARAVRGLFDRLDRFNAGHVPGKPWRTLLAPLPGGPVFVPLGDATPMQADLNAAINIALRGIAAPDRHDIHHRLRAENKKRILSLRLGTQREKARWPGGAPAVTLSTPNNGASPEDSDALPERVSNLFVDIAGVANFERVTIEGVSQKFATGRGLWASVKQRAWNRVARLNETVTDNNRNEEEDDIPM

SEQ ID NO: 57 - protein, 1132 aa, Tuberibacillus calidus
MATKSFILKMKTKNNPQLRLSLWKTHELFNFGVAYYMDLLSLFRQKDLYMHNDEDPDHPVVLKKEEIQERLWMKVRETQQKNGFHGEVSKDEVLETLRALYEELVPSAVGKSGEANQISNKYLYPLTDPASQSGKGTANSGRKPRWKKLKEAGDPSWKDAYEKWEKERQEDPKLKILAALQSFGLIPLFRPFTENDHKAVISVKWMPKSKNQSVRKFDKDMFNQAIERFLSWESWNEKVAEDYEKTVSIYESLQKELKGISTKAFEIMERVEKAYEAHLREITFSNSTYRIGNRAIRGWTEIVKKWMKLDPSAPQGNYLDVVKDYQRRHPRESGDFKLFELLSRPENQAAWREYPEFLPLYVKYRHAEQRMKTAKKQATFTLCDPIRHPLWVRYEERSGTNLNKYRLIMNEKEKVVQFDRLICLNADGHYEEQEDVTVPLAPSQQFDDQIKFSSEDTGKGKHNFSYYHKGINYELKGTLGGARIQFDREHLLRRQGVKAGNVGRIFLNVTLNIEPMQPFSRSGNLQTSVGKALKVYVDGYPKVVNFKPKELTEHIKESEKNTLTLGVESLPTGLRVMSVDLGQRQAAAISIFEVVSEKPDDNKLFYPVKDTDLFAVHRTSFNIKLPGEKRTERRMLEQQKRDQAIRDLSRKLKFLKNVLNMQKLEKTDEREKRVNRWIKDREREEENPVYVQEFEMISKVLYSPHSVWVDQLKSIHRKLEEQLGKEISKWRQSISQGRQGVYGISLKNIEDIEKTRRLLFRWSMRPENPGEVKQLQPGERFAIDQQNHLNHLKDDRIKKLANQIVMTALGYRYDGKRKKWIAKHPACQLVLFEDLSRYAFYDERSRLENRNLMRWSRREIPKQVAQIGGLYGLLVGEVGAQYSSRFHAKSGAPGIRCRVVKEHELYITEGGQKVRNQKFLDSLVENNIIEPDDARRLEPGDLIRDQGGDKFATLDERGELVITHADINAAQNLQKRFWTRTHGLYRIRCESREIKDAVVLVPSDKDQKEKMENLFGIGYLQPFKQENDVYKWVKGEKIKGKKTSSQSDDKELVSEILQEASVMADELKGNRKTLFRDPSGYVFPKDRWYTGGRYFGTLEHLLKRKLAERRLFDGGSSRRGLFNGTDSNTNVE

SEQ ID NO: 58 - protein, 658 aa, Methylobacterium nodulans
MLTKQDKQQKITYCTNMNEVFEAKLGSADLLLNWDHLRGRIRDRVDAGDIGSAFLKLALDVAHVLPDGVDDQLARAAFHFQSAKGAKSKHADSVQAGLRVLSIDLGVRSFATCSVFELKDTAPTTGVAFPLAEFRLWAVHERSFTLELPGENVGAAGQQWRAQADAELRQLRGGLNRHRQLLRAATVQKGERDAYLTDLREAWSAKELWPFEASLLSELERCSTVADPLWQDTCKRAARLYRTEFGAVVSEWRSRTRSREDRKYAGKSMWSVQHLTDVRRFLQSWSLAGRASGDIRRLDRERGGVFAKDLLDHIDALKDDRLKTGADLIVQAARGFQRNEFGYWVQKHAPCHVILFEDLSRYRMRTDRPRRENSQLMQWAHRGVPDMVGMQGEIYGIQDRRDPDSARKHARQPLAAFCLDTPAAFSSRYHASTMTPGIRCHPLRKREFEDQGFLELLKRENEGLDLNGYKPGDLVPLPGGEVFVCLNANGLSRIHADINAAQNLQRRFWTQHGDAFRLPCGKSAVQGQIRWAPLSMGKRQAGALGGFGYLEPTGHDSGSCQWRKTTEAEWRRLSGAQKDRDEAAAAEDEELQGLEEELLERSGERVVFFRDPSGVVLPTDLWFPSAAFWSIVRAKTVGRLRSHLDAQAEASYAVAAGL

SEQ ID NO: 59 - protein, 864 aa, Brevibacillus sp. CF112
MPKILRGHKWISLLEQYEENRERELRENMTAANDKYRITKRQMKGWNELYELWSTFPASASHEQYKEALKRVQQRLRGRFGDAHFFQYLMEEKNRLIWKGNPQRIHYFVARNELTKRLEEAKQSATMTLPNARKHPLWVRFDARGGNLQDYYLTAEADKPRSRRFVTFSQLIWPSESGWMEKKDVEVELALSRQFYQQVKLLKNDKGKQKIEFKDKGSGSTFNGHLGGAKLQLERGDLEKEEKNFEDGEIGSVYLNVVIDFEPLQEVKNGRVQAPYGQVLQLIRRPNEFPKVTTYKSEQLVEWIKASPQHSAGVESLASGFRVMSIDLGLRAAAATSIFSVEESSDKNAADFSYWIEGTPLVAVHHRSYMLRLPGEQVEKQVMEKRDERFQLHQRVKFQIRVLAQIMRMANKQYGDRWDELDSLKQAVEQKKSPLDQTDRTFWEGIVCDLTKVLPRNEADWEQAVVQIHRKAEEYVGKAVQAWRKRFAADERKGIAGLSMWNIEELEGLRKLLISWSRRSRNPQEVNRFERGHTSHQRLLTHIQNVKEDRLKQLSHAIVMTALGYVYDERKQEWCAEYPACQVILFENLSQYRSNLDRSTKENSTLMKWAHRSIPKYVHMQAEPYGIQIGDVRAEYSSRFYAKTGTPGIRCKKVRGQDLQGRRFENLQKRLVNEQFLTEEQVKQLRPGDIVPDDSGELFMTLTDGSGSKEVVFLQADINAAHNLQKRFWQRYNELFKVSCRVIVRDEEEYLVPKTKSVQAKLGKGLFVKKSDTAWKDVYVWDSQAKLKGKTTFTEESESPEQLEDFQEIIEEAEEAKGTYRTLFRDPSGVFFPESVWYPQKDFWGEVKRKLYGKLRERFLTKAR

SEQ ID NO: 60 - protein, 1108 aa, Bacillus sp. NSP2.1
MAIRSIKLKLKTHTGPEAQNLRKGIWRTHRLLNEGVAYYMKMLLLFRQESTGERPKEELQEELICHIREQQQRNQADKNTQALPLDKALEALRQLYELLVPSSVGQSGDAQIISRKFLSPLVDPNSEGGKGTSKAGAKPTWQKKKEANDPTWEQDYEKWKKRREEDPTASVITTLEEYGIRPIFPLYTNTVTDIAWLPLQSNQFVRTWDRDMLQQAIERLLSWESWNKRVQEEYAKLKEKMAQLNEQLEGGQEWISLLEQYEENRERELRENMTAANDKYRITKRQMKGWNELYELWSTFPASASHEQYKEALKRVQQRLRGRFGDAHFFQYLMEEKNRLIWKGNPQRIHYFVARNELTKRLEEAKQSATMTLPNARKHPLWVRFDARGGNLQDYYLTAEADKPRSRRFVTFSQLIWPSESGWMEKKDVEVELALSRQFYQQVKLLKNDKGKQKIEFKDKGSGSTFNGHLGGAKLQLERGDLEKEEKNFEDGEIGSVYLNVVIDFEPLQEVKNGRVQAPYGQVLQLIRRPNEFPKVTTYKSEQLVEWIKASPQHSAGVESLASGFRVMSIDLGLRAAAATSIFSVEESSDKNAADFSYWIEGTPLVAVHQRSYMLRLPGEQVEKQVMEKRDERFQLHQRVKFQIRVLAQIMRMANKQYGDRWDELDSLKQAVEQKKSPLDQTDRTFWEGIVCDLTKVLPRNEADWEQAVVQIHRKAEEYVGKAVQAWRKRFAADERKGIAGLSMWNIEELEGLRKLLISWSRRTRNPQEVNRFERGHTSHQRLLTHIQNVKEDRLKQLSHAIVMTALGYVYDERKQEWCAEYPACQVILFENLSQYRSNLDRSTKENSTLMKWAHRSIPKYVHMQAEPYGIQIGDVRAEYSSRFYAKTGTPGIRCKKVRGQDLQGRRFENLQKRLVNEQFLTEEQVKQLRPGDIVPDDSGELFMTLTDGSGSKEVVFLQADINAAHNLQKRFWQRYNELFKVSCRVIVRDEEEYLVPKTKSVQAKLGKGLFVKKSDTAWKDVYVWDSQAKLKGKTTFTEESESPEQLEDFQEIIEEAEEAKGTYRTLFRDPSGVFFPESVWYPQKDFWGEVKRKLYGKLRERFLTKAR

SEQ ID NO: 61 - protein, 1489 aa, Desulfatirhabdium butyrativorans
MPLSNNPPVTQRAYTLRLRGADPSDLSWREALWHTHEAVNKGAKVFGDWLLTLRGGLDHTLADTKVKGGKGKPDRDPTPEERKARRILLALSWLSVESKLGAPSSYIVASGDEPAKDRNDNVVSALEEILQSRKVAKSEIDDWKRDCSASLSAAIRDDAVWVNRSKVFDEAVKSVGSSLTREEAWDMLERFFGSRDAYLTPMKDPEDKSSETEQEDKAKDLVQKAGQWLSSRYGTSEGADFCRMSDIYGKIAAWADNASQGGSSTVDDLVSELRQHFDTKESKATNGLDWIIGLSSYTGHTPNPVHELLRQNTSLNKSHLDDLKKKANTRAESCKSKIGSKGQRPYSDAILNDVESVCGFTYRVDKDGQPVSVADYSKYDVDYKWGTARHYIFAVMLDHAARRISLAHKWIKRAEAERHKFEEDAKRIANVPARAREWLDSFCKERSVTSGAVEPYRIRRRAVDGWKEVVAAWSKSDCKSTEDRIAAARALQDDSEIDKFGDIQLFEALAEDDALCVWHKDGEATNEPDFQPLIDYSLAIEAEFKKRQFKVPAYRHPDELLHPVFCDFGKSRWKINYDVHKNVQAPFYRGLCLTLWTGSEIKPVPLCWQSKRLTRDLALGNNHRNDAASAVTRADRLGRAASNVTKSDMVNITGLFEQADWNGRLQAPRQQLEAIAVVRDNPRLSEQERNLRMCGMIEHIRWLVTFSVKLQPQGPWCAYAEQHGLNTNPQYWPHADTNRDRKVHARLILPRLPGLRVLSVDLGHRYAAACAVWEAVNTETVKEACQNVGRDMPKEHDLYLHIKVKKQGIGKQTEVDKTTIYRRIGADTLPDGRPHPAPWARLDRQFLIKLQGEEKDAREASNEEIWALHQMECKLDRTKPLIDRLIASGWGLLKRQMARLDALKELGWIPAPDSSENLSREDGEAKDYRESLAVDDLMFSAVRTLRLALQRHGNRARIAYYLISEVKIRPGGIQEKLDENGRIDLLQDALALWHELFSSPGWRDEAAKQLWDSRIATLAGYKAPEENGDNVSDVAYRKKQQVYREQLRNVAKTLSGDVITCKELSDAWKERWEDEDQRWKKLLRWFKDWVLPSGTQANNATIRNVGGLSLSRLATITEFRRKVQVGFFTRLRPDGTRHEIGEQFGQKTLDALELLREQRVKQLASRIAEAALGIGSEGGKGWDGGKRPRQRINDSRFAPCHAVVIENLANYRPDETRTRLENRRLMTWSASKVHKYLSEACQLNGLYLCTVSAWYTSRQDSRTGAPGIRCQDVSVREFMQSPFWRKQVKQAEAKHDENKGDARERFLCELNKTWKAKTPAEWKKAGFVRIPLRGGEIFVSADSKSPSAKGIHADLNAAANIGLRALTDPDWPGKWWYVPCDPVSFESKMDYVKGCAAVKVGQPLRQPAQTNADGAASKIRKGKKNRTAGTSKEKVYLWRDISAFPLESNEIGEWKETSAYQNDVQYRVIRMLKEHIKSLDNRTGDNVEG

SEQ ID NO: 62 - protein, 1228 aa, Lachnospiraceae bacterium ND2006
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 63 - protein, 1307 aa, Acidaminococcus sp. Bv3L6
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLANLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 64 - protein, 1300 aa, Francisella novicida U112
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFQDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 65 - protein, 1246 aa, Porphyromonas macacae
MKTQHFFEDFTSLYSLSKTIRFELKPIGKTLENIKKNGLIRRDEQRLDDYEKLKKVIDEYHEDFIANILSSFSFSEEILQSYIQNLSESEARAKIEKTMRDTLAKAFSEDERYKSIFKKELVKKDIPVWCPAYKSLCKKFDNFTTSLVPFHENRKNLYTSNEITASIPYRIVHVNLPKFIQNIEALCELQKKMGADLYLEMMENLRNVWPSFVKTPDDLCNLKTYNHLMVQSSISEYNRFVGGYSTEDGTKHQGINEWINIYRQRNKEMRLPGLVFLHKQILAKVDSSSFISDTLENDDQVFCVLRQFRKLFWNTVSSKEDDAASLKDLFCGLSGYDPEAIYVSDAHLATISKNIFDRWNYISDAIRRKTEVLMPRKKESVERYAEKISKQIKKRQSYSLAELDDLLAHYSEESLPAGFSLLSYFTSLGGQKYLVSDGEVILYEEGSNIWDEVLIAFRDLQVILDKDFTEKKLGKDEEAVSVIKKALDSALRLRKFFDLLSGTGAEIRRDSSFYALYTDRMDKLKGLLKMYDKVRNYLTKKPYSIEKFKLHFDNPSLLSGWDKNKELNNLSVIFRQNGYYYLGIMTPKGKNLFKTLPKLGAEEMFYEKMEYKQIAEPMLMLPKVFFPKKTKPAFAPDQSVVDIYNKKTFKTGQKGFNKKDLYRLIDFYKEALTVHEWKLFNFSFSPTEQYRNIGEFFDEVREQAYKVSMVNVPASYIDEAVENGKLYLFQIYNKDFSPYSKGIPNLHTLYWKALFSEQNQSRVYKLCGGGELFYRKASLHMQDTTVHPKGISIHKKNLNKKGETSLFNYDLVKDKRFTEDKFFFHVPISINYKNKKITNVNQMVRDYIAQNDDLQIIGIDRGERNLLYISRIDTRGNLLEQFSLNVIESDKGDLRTDYQKILGDREQERLRRRQEWKSIESIKDLKDGYMSQVVHKICNMVVEHKAIVVLENLNLSFMKGRKKVEKSVYEKFERMLVDKLNYLVVDKKNLSNEPGGLYAAYQLTNPLFSFEELHRYPQSGILFFVDPWNTSLTDPSTGFVNLLGRINYTNVGDARKFFDRFNAIRYDGKGNILFDLDLSRFDVRVETQRKLWTLTTFGSRIAKSKKSGKWMVERIENLSLCFLELFEQFNIGYRVEKDLKKAILSQDRKEFYVRLIYLFNLMMQIRNSDGEEDYILSPALNEKNLQFDSRLIEAKDLPVDADANGAYNVARKGLMVVQRIKRGDHESIHRIGRAQWLRYVQEGIVE

SEQ ID NO: 66 - protein, 1373 aa, Moraxella bovoculi 237
MLFQDFTHLYPLSKTVRFELKPIDRTLEHIHAKNFLSQDETMADMHQKVKVILDDYHRDFIADMMGEVKLTKLAEFYDVYLKFRKNPKDDELQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFDKINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADKDYFEFHIDYAKFTDKAKNSRQIWTICSHGDKRYVYDKTANQNKGAAKGINVNDELKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 67 - protein, 1251 aa, Moraxella bovoculi
MLFQDFTHLYPLSKTVRFELKPIGRTLEHIHAKNFLSQDETMADMYQKVKVILDDYHRDFIADMMGEVKLTKLAEFYDVYLKFRKNPKDDELQKQLKDLQAVLRKESVKPIGNGGKYKAGHDRLFGAKLFKDGKELGDLAKFVIAQEGKSSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNRIIGEVNGYTNKHNQICHKSERIAKLRPLHKQILSDGMGVSFLPSKFADDSEMYQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIKHHTARHDDESVQAGKLGQYFKHGLAGVDNPIQKIHNNHSTIKGFLERERPAGERALPKIKSGKNPEMTQLRQLKELLDNALNVAHFAKLLMTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKNVYQKMIYKLLPGPNKMLPKVFFAKSNLGYYNPSAELLDKYAQGTHKKGDNFNLKDCHALIDFFKAGINKHPEWQHFGFKFSPTSSYRDLSDFYREVEPQGYQVKFVDINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQVTTPYHKILDKREIERLNARVGWGEIETIKELKSGYLSHVVHQISQLMLKHNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQVFFGKFDKICYNADKDYFEFHIDYAKFTDKAKNSRQIWTICSHGDKRYVYDKTANQNKGAAKGINVNDELKSLFARHHINDKQPNLVMDICQNNDKEFHKSLMYLLKTLLALRYSNASSDEDFILSPVANDGGVFFNSALADDTQPQNADANGAYHIALKGLWLLNELKDSDDLNKVKLAIDNQTWLNFAQNR

SEQ ID NO: 68 - protein, 1261 aa, Moraxella bovoculi
MLFQDFTHLYPLSKTVRFELKPIGKTLEHIHAKNFLNQDETMADMYQKVKAILDDYHRDFIADMMGEVKLTKLAEFYDVYLKFRKNPKDDGLQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILATIKQKHSALYDQIINELTASGLDVSLASHLDGYHKLLTQEGITAYNTLLGGISGEAGSRKIQGINELINSHHNQHCHKSERIAKLRPLHKQILSDGMGVSFLPSKFADDSEVCQAVNEFYRHYADVFAKVQSLFDGFDDYQKDGIYVEYKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSDKSPEIRQLKELLDNALNVAHFAKLLTTKTTLHNQDGNFYGEFGALYDELAKIATLYNKVRDYLSQKPFSTEKYKLNFGNPTLLNGWDLNKEKDNFGVILQKDGCYYLALLDKAHKKVFDNAPNTGKSVYQKMIYKLLPGPNKMLPKVFFAKSNLDYYNPSAELLDKYAQGTHKKGDNFNLKDCHALIDFFKAGINKHPEWQHFGFKFSPTSSYQDLSDFYREVEPQGYQVKFVDINADYINELVEQGQLYLFQIYNKDFSPKAHGKPNLHTLYFKALFSEDNLVNPIYKLNGEAEIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQMTTPYHKILDKREIERLNARVGWGEIETIKELKSGYLSHVVHQISQLMLKYNAIVVLEDLNFGFKRGCFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADRGYFEFHIDYAKFNDKAKNSRQIWKICSHGDKRYVYDKTANQNKGATIGVNVNDELKSLFTRYHINDKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 69 - protein, 1298 aa, Thiomicrospira sp. XS5
MTKTFDSEFFNLYSLQKTVRFELKPVGETASFVEDFKNEGLKRVVSEDERRAVDYQKVKEIIDDYHRDFIEESLNYFPEQVSKDALEQAFHLYQKLKAAKVEEREKALKEWEALQKKLREKVVKCFSDSNKARFSRIDKKELIKEDLINWLVAQNREDDIPTVETFNNFTTYFTGFHENRKNIYSKDDHATAISFRLIHENLPKFFDNVISFNKLKEGFPELKFDKVKEDLEVDYDLKHAFEIEYFVNFVTQAGIDQYNYLLGGKTLEDGTKKQGMNEQINLFKQQQTRDKARQIPKLIPLFKQILSERTESQSFIPKQFESDQELFDSLQKLHNNCQDKFTVLQQAILGLAEADLKKVFIKTSDLNALSNTIFGNYSVFSDALNLYKESLKTKKAQEAFEKLPAHSIHDLIQYLEQFNSSLDAEKQQSTDTVLNYFIKTDELYSRFIKSTSEAFTQVQPLFELEALSSKRRPPESEDEGAKGQEGFEQIKRIKAYLDTLMEAVHFAKPLYLVKGRKMIEGLDKDQSFYEAFEMAYQELESLIIPIYNKARSYLSRKPFKADKFKINFDNNTLLSGWDANKETANASILFKKDGLYYLGIMPKGKTFLFDYFVSSEDSEKLKQRRQKTAEEALAQDGESYFEKIRYKLLPGASKMLPKVFFSNKNIGFYNPSDDILRIRNTASHTKNGTPQKGHSKVEFNLNDCHKMIDFFKSSIQKHPEWGSFGFTFSDTSDFEDMSAFYREVENQGYVISFDKIKETYIQSQVEQGNLYLFQIYNKDFSPYSKGKPNLHTLYWKALFEEANLNNVVAKLNGEAEIFFRRHSIKASDKVVHPANQAIDNKNPHTEKTQSTFEYDLVKDKRYTQDKFFFHVPISLNFKAQGVSKFNDKVNGFLKGNPDVNIIGIDRGERHLLYFTVVNQKGEILVQESLNTLMSDKGHVNDYQQKLDKKEQERDAARKSWTTVENIKELKEGYLSHVVHKLAHLIIKYNAIVCLEDLNFGFKRGRFKVEKQVYQKFEKALIDKLNYLVFKEKELGEVGHYLTAYQLTAPFESFKKLGKQSGILFYVPADYTSKIDPTTGFVNFLDLRYQSVEKAKQLLSDFNAIRFNSVQNYFEFEIDYKKLTPKRKVGTQSKWVICTYGDVRYQNRRNQKGHWETEEVNVTEKLKALFASDSKTTTVIDYANDDNLIDVILEQDKASFFKELLWLLKLTMTLRHSKIKSEDDFILSPVKNEQGEFYDSRKAGEVWPKDADANGAYHIALKGLWNLQQINQWEKGKTLNLAIKNQDWFSFIQEKPYQE

SEQ ID NO: 70 - protein, 1206 aa, Butyrivibrio sp NC3005
MYYQNLTKKYPVSKTIRNELIPIGKTLENIRKNNILESDVKRKQDYEHVKGIMDEYHKQLINEALDNYMLPSLNQAAEIYLKKHVDVEDREEFKKTQDLLRREVTGRLKEHENYTKIGKKDILDLLEKLPSISEEDYNALESFRNFYTYFTSYNKVRENLYSDEEKSSTVAYRLINENLPKFLDNIKSYAFVKAAGVLADCIEEEEQDALFMVETFNMTLTQEGIDMYNYQIGKVNSAINLYNQKNHKVEEFKKIPKMKVLYKQILSDREEVFIGEFKDDETLLSSIGAYGNVLMTYLKSEKINIFFDALRESEGKNVYVKNDLSKTTMSNIVFGSWSAFDELLNQEYDLANENKKKDDKYFEKRQKELKKNKSYTLEQMSNLSKEDISPIENYIERISEDIEKICIYNGEFEKIVVNEHDSSRKLSKNIKAVKVIKDYLDSIKELEHDIKLINGSGQELEKNLVVYVGQEEALEQLRPVDSLYNLTRNYLTKKPFSTEKVKLNFNKSTLLNGWDKNKETDNLGILFFKDGKYYLGIMNTTANKAFVNPPAAKTENVFKKVDYKLLPGSNKMLPKVFFAKSNIGYYNPSTELYSNYKKGTHKKGPSFSIDDCHNLIDFFKESIKKHEDWSKFGFEFSDTADYRDISEFYREVEKQGYKLTFTDIDESYINDLIEKNELYLFQIYNKDFSEYSKGKLNLHTLYFMMLFDQRNLDNVVYKLNGEAEVFYRPASIAENELVIHKAGEGIKNKNPNRAKVKETSTFSYDIVKDKRYSKYKFTLHIPITMNFGVDEVRRFNDVINNALRTDDNVNVIGIDRGERNLLYVVVINSEGKILEQISLNSIINKEYDIETNYHALLDEREDDRNKARKDWNTIENIKELKTGYLSQVVNVVAKLVLKYNAIICLEDLNFGFKRGRQKVEKQVYQKFEKMLIEKLNYLVIDKSREQVSPEKMGGALNALQLTSKFKSFAELGKQSGIIYYVPAYLTSKIDPTTGFVNLFYIKYENIEKAKQFFDGFDFIRFNKKDDMFEFSFDYKSFTQKACGIRSKWIVYTNGERIIKYPNPEKNNLFDEKVINVTDEIKGLFKQYRIPYENGEDIKEIIISKAEADFYKRLFRLLHQTLQMRNSTSDGTRDYIISPVKNDRGEFFCSEFSEGTMPKDADANGAYNIARKGLWVLEQIRQKDEGEKVNLSMTNAEWLKYAQLHLL

SEQ ID NO: 71 - protein, 1129 aa, Alicyclbacillus acidoterrestris; identical to SEQ ID NO: 52

SEQ ID NO: 72 - RNA, 20 nt, Artificial Sequence
  crRNA repeat LbCas12a
aauuucuacuaaguguagau

SEQ ID NO: 73 - RNA, 19 nt, Artificial Sequence
  crRNA repeast AsCas12a
aauuucuacucuuguagau

SEQ ID NO: 74 - RNA, 19 nt, Artificial Sequence
  crRNA repeast FnCas12a
aauuucuacuguuguagau

SEQ ID NO: 75 - RNA, 19 nt, Artificial Sequence
  crRNA repeat PmCas12a
aauuucuacuauuguagau

SEQ ID NO: 76 - RNA, 20 nt, Artificial Sequence
  crRNA repeat MbCas12a/ Mb2Cas12a/Mb3Cas12a
aauuucuacuguuuguagau

SEQ ID NO: 77 - RNA, 19 nt, Artificial Sequence
  crRNA repeat TsCas12a
aauuucuacuguuguagau

SEQ ID NO: 78 - RNA, 19 nt, Artificial Sequence
  crRNA repeast BsCas12a
aauuucuacuauuguagau

SEQ ID NO: 79 - RNA, 91 nt, Artificial Sequence
  AacCas12b single guide RNA
gucuagaggacagaauuuuucaacgggugugccaauggccacuuuccagguggcaaagcccguugagcuucucaaaucugagaaguggcac

SEQ ID NO: 80 - protein, 986 aa, Deltaproteobacteria bacterium
MEKRINKIRKKLSADNATKPVSRSGPMKTLLVRVMTDDLKKRLEKRRKKPEVMPQVISNNAANNLRMLLDDYTKMKEAILQVYWQEFKDDHVGLMCKFAQPASKKIDQNKLKPEMDEKGNLTTAGFACSQCGQPLFVYKLEQVSEKGKAYTNYFGRCNVAEHEKLILLAQLKPEKDSDEAVTYSLGKFGQRALDFYSIHVTKESTHPVKPLAQIAGNRYASGPVGKALSDACMGTIASFLSKYQDIIIEHQKVVKGNQKRLESLRELAGKENLEYPSVTLPPQPHTKEGVDAYNEVIARVRMWVNLNLWQKLKLSRDDAKPLLRLKGFPSFPVVERRENEVDWWNTINEVKKLIDAKRDMGRVFWSGVTAEKRNTILEGYNYLPNENDHKKREGSLENPKKPAKRQFGDLLLYLEKKYAGDWGKVFDEAWERIDKKIAGLTSHIEREEARNAEDAQSKAVLTDWLRAKASFVLERLKEMDEKEFYACEIQLQKWYGDLRGNPFAVEAENRVVDISGFSIGSDGHSIQYRNLLAWKYLENGKREFYLLMNYGKKGRIRFTDGTDIKKSGKWQGLLYGGGKAKVIDLTFDPDDEQLIILPLAFGTRQGREFIWNDLLSLETGLIKLANGRVIEKTIYNKKIGRDEPALFVALTFERREVVDPSNIKPVNLIGVDRGENIPAVIALTDPEGCPLPEFKDSSGGPTDILRIGEGYKEKQRAIQAAKEVEQRRAGGYSRKFASKSRNLADDMVRNSARDLFYHAVTHDAVLVFENLSRGFGRQGKRTFMTERQYTKMEDWLTAKLAYEGLTSKTYLSKTLAQYTSKTCSNCGFTITTADYDGMLVRLKKTSDGWATTLNNKELKAEGQITYYNRYKRQTVEKELSAELDRLSEESGNNDISKWTKGRRDEALFLLKKRFSHRPVQEQFVCLDCGHEVHADEQAALNIARSWLFLNSNSTEFKSYKSGKQPFVGAWQAFYKRRLKEVWKPNA

SEQ ID NO: 81 - protein, 978 aa, Planctomycetes bacterium
MQEIKRINKIRRRLVKDSNTKKAGKTGPMKTLLVRVMTPDLRERLENLRKKPENIPQPISNTSRANLNKLLTDYTEMKKAILHVYWEEFQKDPVGLMSRVAQPAPKNIDQRKLIPVKDGNERLTSSGFACSQCCQPLYVYKLEQVNDKGKPHTNYFGRCNVSEHERLILLSPHKPEANDELVTYSLGKFGQRALDFYSIHVTRESNHPVKPLEQIGGNSCASGPVGKALSDACMGAVASFLTKYQDIILEHQKVIKKNEKRLANLKDIASANGLAFPKITLPPQPHTKEGIEAYNNVVAQIVIWVNLNLWQKLKIGRDEAKPLQRLKGFPSFPLVERQANEVDWWDMVCNVKKLINEKKEDGKVFWQNLAGYKRQEALLPYLSSEEDRKKGKKFARYQFGDLLLHLEKKHGEDWGKVYDEAWERIDKKVEGLSKHIKLEEERRSEDAQSKAALTDWLRAKASFVIEGLKEADKDEFCRCELKLQKWYGDLRGKPFAIEAENSILDISGFSKQYNCAFIWQKDGVKKLNLYLIINYFKGGKLRFKKIKPEAFEANRFYTVINKKSGEIVPMEVNFNFDDPNLIILPLAFGKRQGREFIWNDLLSLETGSLKLANGRVIEKTLYNRRTRQDEPALFVALTFERREVLDSSNIKPMNLIGIDRGENIPAVIALTDPEGCPLSRFKDSLGNPTHILRIGESYKEKQRTIQAAKEVEQRRAGGYSRKYASKAKNLADDMVRNTARDLLYYAVTQDAMLIFENLSRGFGRQGKRTFMAERQYTRMEDWLTAKLAYEGLPSKTYLSKTLAQYTSKTCSNCGFTITSADYDRVLEKLKKTATGWMTTINGKELKVEGQITYYNRYKRQNVVKDLSVELDRLSEESVNNDISSWTKGRSGEALSLLKKRFSHRPVQEKFVCLNCGFETHADEQAALNIARSWLFLRSQEYKKYQTNKTTGNTDKRAFVETWQSFYRKKLKEVWKPAV

(19) United States
(12) Patent Application Publication
(10) Pub. No.: US 2023/0159986 A1
(43) Pub. Date: May 25, 2023

(54) METHODS FOR DETECTING AND SEQUENCING A TARGET NUCLEIC ACID

(71) Applicants: The Regents of the University of California, Oakland, CA (US); Mammoth Biosciences, Inc., Brisbane, CA (US)

(72) Inventors: Charles Chiu, Oakland, CA (US); Janice Sha Chen, Brisbane, CA (US); Xin Miao, Brisbane, CA (US)

(21) Appl. No.: 17/920,571

(22) PCT Filed: Apr. 22, 2021
(86) PCT No.: PCT/US2021/028636

§ 371 Date: Oct. 21, 2022

Related U.S. Application Data
(60) Provisional application No. 63/014,076, filed on Apr. 22, 2020.

(51) Int. Cl. C12Q 1/6806; C12Q 1/689; C12Q 1/6893; C12Q 1/6895

(52) U.S. Cl. CPC C12Q 1/6806; C12Q 1/689; C12Q 1/6893; C12Q 1/6895

(57) ABSTRACT

The disclosure provides methods for characterizing a target DNA present in a sample. The methods involve contacting the sample with one or more universal primers to amplify target DNA; contacting the amplified target DNA with a type V CRISPR/Cas effector protein and one or more guide RNAs, where the contacting generates a cleavage product comprising a 5′ overhang; and ligating a double-stranded nucleic acid adapter to the cleavage product, to generate a ligation product. The ligation product includes the target DNA, which can be sequenced. The sample can be subjected to one or more amplification steps prior to the contacting step, with primers that provide for amplification of nucleic acids of, e.g., specific pathogens, categories of pathogens, two or more different pathogens, or two or more different categories of pathogens.

### CROSS REFERENCE TO RELATED APPLICATIONS

This application claims priority under 35 U.S.C. § 119 from Provisional Application Ser. No. 63/014,076, filed Apr. 22, 2020, the disclosures of which are incorporated herein by reference for all purposes.

### STATEMENT REGARDING FEDERALLY SPONSORED RESEARCH

This invention was made with Government support under Grant No: W81XWH-17-1-0681, awarded by the Department of Defense and Grant No. R33AI120977, awarded by the National Institutes of Health. The Government has certain rights in the invention.

### INCORPORATION BY REFERENCE OF SEQUENCE LISTING

Accompanying this filing is a Sequence Listing entitled “Sequence-Listing_ST25.txt”, created on Apr. 22, 2021 and having 233,847 bytes of data, machine formatted on IBM-PC, MS-Windows operating system. The sequence listing is hereby incorporated herein by reference in its entirety for all purposes.

### FIELD OF THE INVENTION

The disclosure relates to the field of genomics and diagnostics, and more particularly to the detection and genomic characterization of microorganisms in a sample.

### BACKGROUND

Bacterial adaptive immune systems employ CRISPRs (clustered regularly interspaced short palindromic repeats) and CRISPR-associated (Cas) proteins for RNA-guided nucleic acid cleavage. The CRISPR-Cas systems thereby confer adaptive immunity in bacteria and archaea via RNA-guided nucleic acid interference. To provide anti-viral immunity, processed CRISPR array transcripts (crRNAs) assemble with Cas protein-containing surveillance complexes that recognize nucleic acids bearing sequence complementarity to the virus derived segment of the crRNAs, known as the spacer.

Class 2 CRISPR-Cas systems are streamlined versions in which a single Cas protein (an effector protein, e.g., a type V Cas effector protein such as Cpf1) bound to RNA is responsible for binding to and cleavage of a targeted sequence. The programmable nature of these minimal systems has facilitated their use as a versatile technology that continues to revolutionize the field of genome manipulation.

### SUMMARY

The disclosure provides a method for characterizing a target DNA present in a sample, the method comprising: amplifying nucleic acids in the sample using at least one universal primer for a particular taxonomic rank of a desired organism to be detected to obtain amplified target DNA; contacting the sample with: a type V CRISPR/Cas effector protein; and one or more guide RNAs, wherein the one or more guide RNAs comprise: i) a region that binds to the type V CRISPR/Cas effector protein; and ii) a guide sequence that hybridizes with the amplified target DNA, and a plurality of detector DNAs; wherein said contacting generates a protospacer adjacent motif (PAM)-distal cleavage product comprising a 5′ overhang; and optionally ligating a double-stranded nucleic acid adapter to the cleavage product, wherein the adapter comprises a 5′overhang that comprises a stretch of from 3 to 15 contiguous nucleotides that are complementary to a contiguous stretch of nucleotides of the same length in the 5′ overhang of the PAM-distal cleavage product, wherein said ligating generates a ligation product comprising the adapter and the PAM-distal cleavage product; and determining the nucleotide sequence of the PAM-distal cleavage product present in the ligation product. In one embodiment, the type V CRISPR/Cas effector protein is a Cas12 protein. In another embodiment, the type V CRISPR/Cas effector protein is a Cas12a (Cpf1) protein. In still another embodiment, the type V CRISPR/Cas effector protein is a Cas12b (C2c1) protein. In yet another embodiment, the type V CRISPR/Cas effector protein is a Cas12d protein. In another embodiment, the type V CRISPR/Cas effector protein is a Cas14a protein. In yet another or further embodiment of any of the foregoing embodiments, the amplified target DNA is single stranded. In yet another or further embodiment of any of the foregoing embodiments, the amplified target DNA is double stranded. In yet another or further embodiment of any of the foregoing embodiments, the amplified target DNA is bacterial DNA. In yet another or further embodiment of any of the foregoing embodiments, the amplified target DNA is mycobacterium DNA. In yet another or further embodiment of any of the foregoing embodiments, the amplified target DNA is Babesia DNA. In yet another or further embodiment of any of the foregoing embodiments, the amplified target DNA is fungal DNA. In yet another or further embodiment of any of the foregoing embodiments, the at least one universal primer is selected from oligonucleotides having the sequence of SEQ ID NOs:1-7, 8, or any two or more of SEQ ID NOs:1-8. In yet another or further embodiment of any of the foregoing embodiments, the method is used to detect bacteria. In yet another or further embodiment of any of the foregoing embodiments, the at least one universal primer is selected from oligonucleotides having the sequence of SEQ ID NOs:9-14, 15, or any two or more of SEQ ID NOs:9-15. In yet another or further embodiment of any of the foregoing embodiments, the method is used to detect babesia. In yet another or further embodiment of any of the foregoing embodiments, the at least one universal primer is selected from oligonucleotides having the sequence of SEQ ID NOs:16-22, 23, or any two or more of SEQ ID NOs:16. In yet another or further embodiment of any of the foregoing embodiments, the method is used to detect mycobacteria. In yet another or further embodiment of any of the foregoing embodiments, the at least one universal primer is selected from oligonucleotides having the sequence of SEQ ID NOs:24-28, 29, or any two or more of SEQ ID NOs:24. In yet another or further embodiment of any of the foregoing embodiments, the composition is used to detect fungi. In yet another or further embodiment of any of the foregoing embodiments, the guide sequence of the guide RNA hybridizes to a sub-taxonomic classification of the particular taxonomic rank in the amplified target DNA. In yet another or further embodiment of any of the foregoing embodiments, comprising contacting the sample with 2 or more guide RNAs, wherein the 2 or more guide RNAs differ from one another in the guide sequence. In a further embodiment, the sample with from 2 to 10 guide RNAs. In yet another or further embodiment of any of the foregoing embodiments, the sample is a cell-free sample. In yet another or further embodiment of any of the foregoing embodiments, the sample is blood, serum, plasma, bronchoalveolar lavage, sputum, urine, cerebrospinal fluid, feces, or a biopsy sample. In yet another or further embodiment of any of the foregoing embodiments, the amplifying comprises isothermal amplification. In yet another or further embodiment of any of the foregoing embodiments, the amplification comprises contacting the sample with 1 or more pairs of forward and reverse primers, wherein at least one primer is selected from primers of Table 1. In yet another or further embodiment of any of the foregoing embodiments, the adapter comprises a 3′ deoxyadenosine overhang. In yet another or further embodiment of any of the foregoing embodiments, wherein sequence determination is carried out by nanopore sequencing. In yet another or further embodiment of any of the foregoing embodiments, the target DNA is present in the sample at a concentration as low as 200 fM. In yet another or further embodiment of any of the foregoing embodiments, the detector DNA is single stranded and does not hybridize with the guide sequence of the guide RNA; and measuring a detectable signal produced by cleavage of the detector DNA by the type V CRISPR/Cas effector protein, thereby detecting the target DNA. In yet another or further embodiment of any of the foregoing embodiments, the detector DNA comprises a fluorescence-emitting dye pair. In yet another or further embodiment of any of the foregoing embodiments, the fluorescence-emitting dye pair is a fluorescence resonance energy transfer (FRET) pair. In yet another or further embodiment of any of the foregoing embodiments, the fluorescence-emitting dye pair is a quencher/fluor pair. In yet another or further embodiment of any of the foregoing embodiments, the detector DNA comprises a modified nucleobase, a modified sugar moiety, and/or a modified nucleic acid linkage.

The disclosure also provides a kit for characterizing a target DNA present in a sample, the system comprising: one or more universal primers or primer pairs provided in Table 1; a type V CRISPR/Cas effector protein; one or more guide RNAs, wherein the one or more guide RNAs comprise: i) a region that binds to the type V CRISPR/Cas effector protein; and ii) a guide sequence that hybridizes with the target DNA; a plurality of detector DNAs; and optionally a double-stranded nucleic acid adapter, wherein the adapter comprises a 5′overhang that comprises a stretch of from 3 to 15 contiguous nucleotides that are complementary to a contiguous stretch of nucleotides of the same length in the 5′ overhang of protospacer adjacent motif(PAM)-distal cleavage product generated by action of the type V CRISPR/Cas effector protein and the one or more guide RNAs on the target DNA. In one embodiment, the kit further comprises one or more reagents for determining the nucleotide sequence of a ligation product formed by ligating the adapter and the PAM-distal cleavage product. In yet another embodiment, the kit further comprises one or more reagents for amplifying the target DNA.

### BRIEF DESCRIPTION OF THE DRAWINGS

FIG. 1 provides a schematic for Cas12 cleavage and adapter ligation for nanopore sequencing. Following Cas12a cleavage and DNA detection (via DETECTR), the PAM-distal product containing a 5′ overhang is released from the complex, which is simultaneously ligated to a “clicker” molecule that can be directly coupled with next-generation sequencing protocols (Oxford Nanopore, Illumina, etc). The “clicker” contains a sequence complementary to the cleaved product for selective ligation and a 3′ dA for adapter ligation.

FIG. 2A-V provides amino acid sequences of various Type V CRISPR/Cas effector proteins (depicted are Cas12b sequences) (FIG. 2A-2J); amino acid sequences of various Type V CRISPR/Cas effector proteins (depicted are Cas12a and Cas12b sequences) (FIG. 2K-2T); and example guide RNA sequences (e.g., crRNA repeat sequences and an example single guide RNA sequence) and example PAM sequences (FIG. 2U and FIG. 2V).

FIG. 3A-B provides amino acid sequences of Type V CRISPR/Cas effector proteins (depicted are Cas12e sequences).

FIG. 4 is a graph showing kinetic curve of the DETECTR reaction

FIG. 5 is a graph that shows the kinetic curve of the DETECTR assay.

FIG. 6 is a graph showing the time to results of the samples in the DETECTR assay.

### DETAILED DESCRIPTION

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4^{th} ed. 2007); Sambrook et al., MOLECULAR CLONING, A LABORATORY MANUAL, Cold Spring Harbor Lab Press (Cold Spring Harbor, N.Y. 1989), both of which are incorporated herein by reference. All patents, patent applications, and publications mentioned herein are incorporated herein by reference in their entireties for all purposes.

The term “a”, “an” or “the” is intended to mean “one or more”, e.g., a pathogen refers to one or more pathogenic microorganisms unless otherwise made clear from the context of the text.

The term “comprise,” and variations thereof such as “comprises” and “comprising,” when preceding the recitation of a step or an element, are intended to mean that the addition of further steps or elements is optional and not excluded.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

As used herein, the term “amplifying” refers to the process of synthesizing nucleic acid molecules that are complementary to one (or both strands) of a template nucleic acid molecule. Amplifying a nucleic acid molecule typically includes denaturing the template nucleic acid, particularly if the template nucleic acid is double-stranded, annealing one or more primers to the template nucleic acid at a temperature that is below the melting temperatures of the primers, and enzymatically elongating from the primers to generate an amplification product. Generally, synthesis initiates at the 3′ end of a primer and proceeds in a 5′ to 3′ direction along the template nucleic acid strand. Amplification typically requires the presence of deoxyribonucleoside triphosphates, a polymerase enzyme (e.g., DNA or RNA polymerase or T7 for in vitro transcription in TMA) and an appropriate buffer and/or co-factors for optimal activity of the polymerase enzyme (e.g., MgCl₂ and/or KCl).

“Binding” as used herein (e.g. with reference to an RNA-binding domain of a polypeptide, binding to a target nucleic acid, and the like) refers to a non-covalent interaction between macromolecules (e.g., between a protein and a nucleic acid; between a guide RNA and a target nucleic acid; and the like). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (Kd) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower Kd.

By “binding domain” it is meant a protein domain that is able to bind non-covalently to another molecule. A binding domain can bind to, for example, an RNA molecule (an RNA-binding domain) and/or a protein molecule (a protein-binding domain). In the case of a protein having a protein-binding domain, it can in some cases bind to itself (to form homodimers, homotrimers, etc.) and/or it can bind to one or more regions of a different protein or proteins.

As used herein, the term “complement thereof” or “complementary” refers to a nucleic acid molecule that is optionally the same length as a target molecule of interest and possesses a structural (e.g., nucleotide) composition that is complementary (i.e., capable of conventional hydrogen base pairing) with the target molecule of interest, unless otherwise specified. Substantial complementarity refers to a nucleic acid molecule that is optionally the same length as the target molecule of interest but is greater than 90% complementary and less than 100% complementary to the target molecule of interest.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

With respect to the term “different taxon of pathogens”, the term is distinct from the “particular taxon of pathogens”. Here, the different taxon of pathogenic microorganisms does not overlap with the particular taxon of pathogens. For example, if a particular taxon of pathogenic microorganisms includes the family of Flavivirus, the different taxon of pathogenic microorganisms does not include Flavivirus but can include another family of viruses, such as Alphaviruses, bacterial, fungal, archaea, algal, protozoan, and/or parasitic pathogens. If the particular taxon of pathogenic microorganisms and different taxon of pathogenic microorganisms are from the same domain (e.g., bacterial domain), the two taxa identified by the method are distinct.

As used herein, the terms “extension”, “extend” or “elongation” when used with respect to nucleic acid molecules refers to a biological process by which additional nucleotides (or nucleotide analogs) are incorporated into nucleic acid molecules. For example, a nucleic acid can be extended by a nucleotide incorporating enzyme, such as a polymerase or reverse transcriptase that typically adds sequentially, a nucleotide to the 3′ terminal end of the nucleic acid molecule (e.g., the freely available 3′-OH group).

As used herein, “hybridization”, “hybridizing”, “anneal” and “annealing”, and the like, refer to a process of combining two complementary (or substantially complementary (e.g., at least 90%) single-stranded DNA or RNA molecules so as to form a double-stranded molecule (DNA/DNA, DNA/RNA, RNA/RNA) through conventional hydrogen base pairing. Hybridization stringency is typically determined by the hybridization temperature and salt concentration of the hybridization buffer; e.g., high temperature and low salt provide high stringency hybridization conditions. Examples of salt concentration ranges and temperature ranges for different hybridization conditions are as follows: high stringency, approximately 0.01 M to approximately 0.05 M salt, hybridization temperature 5° C. to 10° C. below Tₘ; moderate stringency, approximately 0.16 M to approximately 0.33 M salt, hybridization temperature 20° C. to 29° C. below Tₘ; and low stringency, approximately 0.33 M to approximately 0.82 M salt, hybridization temperature 40° C. to 48° C. below Tₘ of duplex nucleic acids is calculated by standard methods well-known in the art (see, e.g., Maniatis, T., et al., Molecular Cloning: A Laboratory Manual, Cold Spring Harbor Laboratory Press: New York (1982); Casey, J., et al., Nucleic Acids Research 4:1539-1552 (1977); Bodkin, D. K., et al., Journal of Virological Methods 10(1):45-52 (1985); Wallace, R. B., et al., Nucleic Acids Research 9(4):879-894 (1981)). Algorithm prediction tools to estimate Tₘ are also publicly available (see, e.g., [http://] [tmcalculator.neb.com]). High stringency conditions for hybridization typically refer to conditions under which a nucleic acid molecule having complementarity (or substantial complementarity, e.g., greater than 90%, 95%, 98%, 99% complementarity) to a target sequence predominantly hybridizes with the target sequence and does not hybridize to non-target or off-target sequences.

In some embodiments, hybridizing refers to the annealing of a primer to a complementary (or substantially complementary (e.g., greater than 90% complementary)) template (or target) RNA or DNA sequence obtained from a pathogen. In another embodiment, hybridizing can include annealing at least one probe to an amplification product (e.g., cDNA molecule) derived from a pathogen. Hybridization conditions typically include a temperature below the melting temperature of the primers or probes to reduced non-specific hybridization of the primers/probes. Accordingly, in some embodiments of the disclosure, hybridization conditions are of moderate stringency or high stringency.

By “hybridizable” or “complementary” or “substantially complementary” it is meant that a nucleic acid (e.g., RNA, DNA) comprises a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine/adenosine) (A) pairing with thymidine/thymidine (T), A pairing with uracil/uridine (U), and guanine/guanosine) (G) pairing with cytosine/cytidine (C). In addition, for hybridization between two RNA molecules (e.g., dsRNA), and for hybridization of a DNA molecule with an RNA molecule (e.g., when a DNA target nucleic acid base pairs with a guide RNA, etc.): G can also base pair with U. For example, G/U base-pairing is partially responsible for the degeneracy (i.e., redundancy) of the genetic code in the context of tRNA anti-codon base-pairing with codons in mRNA. Thus, in the context of this disclosure, a G (e.g., of a protein-binding segment (e.g., dsRNA duplex) of a guide RNA molecule; of a target nucleic acid (e.g., target DNA) base pairing with a guide RNA) is considered complementary to both a U and to C. For example, when a G/U base-pair can be made at a given nucleotide position of a protein-binding segment (e.g., dsRNA duplex) of a guide RNA molecule, the position is not considered to be non-complementary, but is instead considered to be complementary.

Hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tₘ) for hybrids of nucleic acids having those sequences. Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more).

It is understood that the sequence of a polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure, a ‘bulge’, and the like). A polynucleotide can comprise 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. The remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

As used herein, the terms “identical” or “percent identity” in the context of two or more nucleic acid sequences, refers to two or more sequences that are the same or have a specified percentage of nucleotides that are the same (i.e., identical), when compared and aligned for maximum correspondence, e.g., as measured using one of the sequence comparison algorithms or by visual inspection. An exemplary algorithm that is suitable for determining percent sequence identity and sequence similarity is the BLAST program, which are described in Altschul et al. (1990) “Basic local alignment search tool” J. Mol. Biol. 215:403-410, Gish et al. (1993) “Identification of protein coding regions by database similarity search” Nature Genet. 3:266-272, Madden et al. (1996) “Applications of network BLAST server” Meth. Enzymol. 266:113-141, Altschul et al. (1997) “Gapped BLAST and PSI-BLAST: a new generation of protein database search programs” Nucleic Acids Res. 25:3389-3402, and Zhang et al. (1997) “PowerBLAST: A new network BLAST application for interactive or automated sequence analysis and annotation” Genome Res. 7:649-656.

Other exemplary multiple sequence alignment computer programs include MAFFT ([https://] [mafft.cbrcjp/alignment/software/]), MUSCLE ([https://] [www.ebi.ac.uk/Tools/msa/muscle/]), and CLUSTALW ([https://] [www.ebi.ac.uk/Tools/msa/clustalw2/]). Percent identity between two nucleic acid sequences is generally calculated using standard default parameters of the various methods or computer programs. A high degree of sequence identity, as used herein, between two nucleic acid molecules is typically at least 90% identity, at least 91% identity, at least 92% identity, at least 93% identity, at least 94% identity, at least 95% identity, at least 96% identity, at least 97% identity, at least 98% identity, at least 99% identity, at least 99.5% identity, or any range of percent identity that includes or is between any two of the foregoing percentages (e.g., between 90% identity and 100% identity, between 95% identity and 98% identity, etc.). A moderate degree of sequence identity, as used herein, between two nucleic acid molecules is typically at least 80% identity, at least 82% identity, at least 83% identity, at least 84% identity, at least 85% identity, at least 86% identity, at least 87% identity, at least 88% identity, at least 89% identity, or any range of percent identity that includes or is between any two of the foregoing percentages (e.g., between 80% identity and 90% identity, between 85% identity and 89% identity, etc.). A low degree of sequence identity, as used herein, between two nucleic acid molecules is typically at least 50% identity, at least 55% identity, at least 60% identity, at least 65% identity, at least 70% identity, at least 75% identity, at least 79% identity, or any range of percent identity that includes or is between any two of the foregoing percentages (e.g., between 50% identity and 70% identity, 55% identity and 75% identity). For example, a sample from a subject, (e.g., suspected of being infected with Zika virus) can have a high degree of sequence identity to a reference taxon of pathogenic microorganisms (e.g., Flavivirus) and a low degree of sequence identity to bacterial pathogenic microorganisms (e.g., Streptococcus, Clostridium, Salmonella and Mycobacterium).

The terms “joining” and “ligation” as used herein, with respect to two polynucleotides, such as an adapter nucleic acid and a PAM-distal cleavage product, refers to the covalent attachment of two separate polynucleotides to produce a single larger polynucleotide with a contiguous backbone. Methods for joining two polynucleotides are known in the art, and include without limitation, enzymatic and non-enzymatic (e.g. chemical) methods. Examples of ligation reactions that are non-enzymatic include the non-enzymatic ligation techniques described in U.S. Pat. Nos. 5,780,613 and 5,476,930, which are incorporated herein by reference. In some cases, an adapter nucleic acid is joined to a target polynucleotide (e.g., a PAM-distal cleavage product) by a ligase, for example a DNA ligase or RNA ligase. Multiple ligases, each having characterized reaction conditions, are known in the art, and include, without limitation NAD+-dependent ligases including tRNA ligase, Taq DNA ligase, Thermus filiformis DNA ligase, Escherichia coli DNA ligase, Tth DNA ligase, Thermus scotoductus DNA ligase (I and II), thermostable ligase, Ampligase thermostable DNA ligase, VanC-type ligase, 9° N DNA Ligase, Tsp DNA ligase, and novel ligases discovered by bioprospecting; ATP-dependent ligases including T4 RNA ligase, T4 DNA ligase, T3 DNA ligase, T7 DNA ligase, Pfu DNA ligase, DNA ligase 1, DNA ligase III, DNA ligase IV, and novel ligases discovered by bioprospecting; and wild-type, mutant isoforms, and genetically engineered variants thereof.

Ligation can be between polynucleotides having hybridizable sequences, such as complementary overhangs. Ligation can also be between two blunt ends. Generally, a 5′ phosphate is utilized in a ligation reaction. The 5′ phosphate can be provided by the target polynucleotide, the adapter oligonucleotide, or both. 5′ phosphates can be added to or removed from polynucleotides to be joined, as needed. Methods for the addition or removal of 5′ phosphates are known in the art, and include without limitation enzymatic and chemical processes. Enzymes useful in the addition and/or removal of 5′ phosphates include kinases, phosphatases, and polymerases. In some cases, both of the two ends joined in a ligation reaction (e.g. an adapter end and a target polynucleotide end) provide a 5′ phosphate, such that two covalent linkages are made in joining the two ends. In some cases, only one of the two ends joined in a ligation reaction (e.g. only one of an adapter end and a target polynucleotide end) provides a 5′ phosphate, such that only one covalent linkage is made in joining the two ends. In some cases, only one strand at one or both ends of a target polynucleotide is joined to an adapter nucleic acid. In some cases, both strands at one or both ends of a target polynucleotide (e.g., a PAM-distal cleavage product) are joined to an adapter nucleic acid. In some cases, 3′ phosphates are removed prior to ligation. In some cases, an adapter nucleic acid is added to only one end of a target polynucleotide (e.g., a PAM-distal cleavage product). When both strands at both ends are joined to an adapter oligonucleotide, joining can be followed by a cleavage reaction that leaves a 5′ overhang that can serve as a template for the extension of the corresponding 3′ end, which 3′ end may or may not include one or more nucleotides derived from the adapter oligonucleotide. In some cases, a target polynucleotide (e.g., a PAM-distal cleavage product) is joined to a first adapter nucleic acid on one end and a second adapter oligonucleotide on the other end. In some cases, two ends of a target polynucleotide are joined to the opposite ends of a single adapter oligonucleotide. In some cases, the target polynucleotide and the adapter nucleic acid to which it is joined comprise blunt ends. In some cases, separate ligation reactions are carried out for each sample, using a different first adapter nucleic acid comprising at least one barcode sequence for each sample, such that no barcode sequence is joined to the target polynucleotides of more than one sample. A target polynucleotide that has an adapter nucleic acid joined to it is considered “tagged” by the joined adapter.

The term “microorganism” or “microbial organism” is used in its broadest sense and includes Gram negative aerobic bacteria, Gram positive aerobic bacteria, Gram negative microaerophillic bacteria, Gram positive microaerophillic bacteria, Gram negative facultative anaerobic bacteria, Gram positive facultative anaerobic bacteria, Gram negative anaerobic bacteria, Gram positive anaerobic bacteria, Gram positive asporogenic bacteria, Actinomycetes, fungal microorganism, protazoan microorganism and the like.

As used herein, a “modified nucleotide” or “nucleotide analog” in the context of an oligonucleotide, primer or probe, refers to incorporation of a non-naturally occurring nucleotide (e.g., a nucleotide other than A, G, T, C or U) within the oligonucleotide, primer or probe, and whereby incorporation of the modified nucleotide or nucleotide analog does not hinder or prevent nucleic acid extension or elongation under suitable amplification conditions. Examples of nucleic acid modifications are described in, e.g., U.S. Pat. No. 6,001,611. Other modified nucleotide substitutions may alter the stability of the oligonucleotide (e.g., modulate its Tm), or provide other desirable features (e.g., nuclease resistance).

As used herein, the terms “nucleic acid”, “polynucleotide” and “oligonucleotide” refer to a polymeric form of nucleotides. The nucleotides may be deoxyribonucleotides (DNA), ribonucleotides (RNA), analogs thereof, or combinations thereof, and may be of any length. Polynucleotides may perform any function and may have any secondary and tertiary structures (e.g., hairpins, stem loop structures). Oligonucleotides refer to polymeric form of nucleotides typically having much shorter lengths than polynucleotides (e.g., ≤50 nt). The terms encompass known analogs of natural nucleotides and nucleotides that are modified in the base, sugar and/or phosphate moieties. Preferably, analogs of a particular nucleotide have the same base-pairing specificity (e.g., an analog of A base pairs with T). An oligonucleotide may comprise one modified nucleotide or multiple modified nucleotides. Examples of modified nucleotides include fluorinated nucleotides, methylated nucleotides, and nucleotide analogs. The nucleotide structure may be modified before or after a polymer is assembled. The terms also encompass nucleic acids comprising modified backbone residues or linkages that are synthetic, naturally occurring, and non-naturally occurring, and have similar binding properties as a reference polynucleotide (e.g., DNA or RNA). Examples of such analogs include, but are not limited to, phosphorothioates, phosphoramidates, methyl phosphonates, chiral-methyl phosphonates, 2-O-methyl ribonucleotides, peptide-nucleic acids (PNAs), Locked Nucleic Acid (LNA) and morpholino structures. Thus, terms “polynucleotide” and “nucleic acid” encompass single-stranded DNA; double-stranded DNA; multi-stranded DNA; single-stranded RNA; double-stranded RNA; multi-stranded RNA; genomic DNA; cDNA; DNA-RNA hybrids; and a polymer comprising purine and pyrimidine bases or other natural, chemically or biochemically modified, non-natural, or derivatized nucleotide bases.

As used herein, the term “pathogen” refers to a virus, bacterium, protozoa, prion, archaea, fungus, algae, parasite, or other microbe (helminth) that causes or induces disease or illness in a subject or that may be found in biological and/or environmental samples. The term includes both the disease-causing organism per se and toxins produced by the pathogen (e.g., Shiga toxins) present in a sample. Detection of a pathogen as set forth in the methods disclosed herein includes detection of a portion of the genome of the pathogen or a nucleic acid molecule that is complementary or substantially complementary (i.e., at least 90% complementary) to a portion of the genome of the pathogen.

With respect to the term “particular taxon of pathogens”, the term refers to classification or taxonomy of pathogens. Accordingly, a “particular taxon of pathogens” can include pathogenic microorganisms classified at various levels of taxonomic rank, e.g., by Realm (Riboviria), Domain/SubRealm (e.g., Bacteria, Arachaea), by Kingdom (e.g., Protista, Fungi, etc.), by Phylum (e.g., Vira, Chlamydiae, etc.), by Class (e.g., Chlamydiales, Parachlamydiales, etc.), by Order (e.g., caudovirales, herpesvirales, ligamenvirales, mononegavirales, etc.), by Family (e.g., Reoviridae, Caliciviridae, Flaviviridae, Orthomyxoviridae, Picornaviridae, Togaviridae, Paramyxoviridae, Bunyaviridae, Rhabdoviridae, Filoviridae, Coronaviridae, Astroviridae, Bornaviridae, Arteriviridae, Hepeviridae, Retroviridae, etc.), or by Genus (e.g., Hepacivirus, flavivirus, pegivirus, pestivirus, etc.). Thus, “a particular taxon of pathogens” refers to a group of related species that share significant properties, but may differ in host range and virulence. An exemplary taxonomic classification system of viruses suitable for use with the disclosure is the international committee on Taxonomy of viruses (ICTV) which organizes viruses based on the structure and composition of viruses. For example, the ICTV database, freely available at [https://]talk.ictvonline.org/ictv-reports/ictv_online_report/ (note the “https” has been bracketed to remove active hyperlinks) classifies viruses as either ssDNA viruses, ssDNA/dsRNA viruses, dsDNA viruses, dsRNA viruses, reverse transcribing DNA and RNA viruses, negative sense RNA viruses and positive sense RNA viruses. For purposes of this disclosure, “a particular taxon of viruses” will refer to a Family or Genus taxonomic level of related viruses. Typically, viral genus names end in the suffix -virus.

Bacteria and fungi are also routinely classified or ranked based on different taxa corresponding to genus, family, and species identification. For example, fungal taxon contemplated by the disclosure include any of the fungal taxon provided in List 1 or List 2. It will be apparent to one of ordinary skill in the art that List 1 and List 2 are not exhaustive and are provided as exemplary lists.

List 1: Fungal Genera:

Anaeromyces, Caecomyces, Allomyces, Entyloma, Diskagma, Blastocladia, Funneliformis, Entylomella, Coelomomyces, Glomus (fungus), Fusidium, Heptameria, Holmiella, Homostegia, Hyalocrea, Hyalosphaera, Hypholoma, Hypobryon, Hysteropsis, Koordersiella, Karschia, Kirschsteiniothelia, Lembosiopeltis, Kullhemia, Kusanobotrys, Leptodothiorella, Lanatosphaera, Lasiodiplodia, Leveillina, Lepidopterella, Lepidostroma, Lollipopaia, Leptosphaerulina, Leptospora, Macrovalsaria, Lichenostigma, Licopolia, Massariola, Lopholeptosphaeria, Maireella, Microdothella, Macroventuria, Microcyclella, Mycoglaena, Melanodothis, Montagnella, Mycoporopsis, Moniliella, Mycopepon, Myriangium, Mycomicrothelia, Mycothyridium, Mytilostoma, Mycosphaerella, Mytilinidion, Neofusicoccum, Myriostigmella, Neocallimastix, Oomyces, Neopeckia, Orpinomyces, Ostreichnion, Ophiosphaerella, Paropodia, Passeriniella, Passerinula, Pedumispora, Peyronellaea, Phaeoacremonium, Phaeocyrtidula, Phaeoglaena, Phaeopeltosphaeria, Phaeoramularia, Phaeosperma, Phaneromyces, Phialophora, Philonectria, Phragmocapnias, Phragmosperma, Piedraia, Piromyces, Placocrea, Placostromella, Plagiostromella, Plejobolus, Pleostigma, Polychaeton, Pseudocercospora, Pseudocryptosporella, Pseudogymnoascus, Pseudothis, Pycnocarpon, Rhytidhysteron, Rhizophagus (fungus), Rhopographus, Rosellinula, Rhytisma, Robillardiella, Roussoëllopsis, Rosenscheldia, Rostafinskia, Sarcopodium, Savulescua, Saksenaeaceae, Scolecobonaria, Scolicotrichum, Schizoparme, Semifissispora, Septoria, Scorias, Sphaceloma, Sphaerellothecium, Spathularia, Stagonosporopsis, Stenella (fungus), Sphaerulina, Stigmina (fungus), Stioclettia, Stigmidium, Sydowia, Tephromela, Stuartella, Teichosporella, Thalloloma, Taeniolella, Thalassoascus, Togninia, Teratosphaeria, Thyrospora, Thyridaria, Yarrowia, Wettsteinina, Valsaria, Ustilaginoidea, Yoshinagella, Wernerella (fungus), and Vismya.

List 2: Fungi Species:

Absidia corymbifera, Absidia ramose, Achorion gallinae, Actinomadura spp., Ajellomyces dermatididis, Aleurisma brasiliensis, Allersheria boydii, Arthroderma spp., Aspergillus flavus, Aspergillus fumigatu, Basidiobolus spp, Blastomyces spp, Cadophora spp, Candida albicans, Cercospora apii, Chrysosporium spp, Cladosporium spp, Cladothrix asteroids, Coccidioides immitis, Cryptococcus albidus, Cryptococcus gattii, Cryptococcus laurentii, Cryptococcus neoformans, Cunninghamella elegans, Dematium wernecke, Discomyces israelii, Emmonsia spp, Emmonsiella capsulate, Endomyces geotrichum, Entomophthora coronate, Epidermophyton floccosum, Filobasidiella neoformans, Fonsecaea spp., Geotrichum candidum, Glenospora khartoumensis, Gymnoascus gypseus, Haplosporangium parvum, Histoplasma, Histoplasma capsulatum, Hormiscium dermatididis, Hormodendrum spp., Keratinomyces spp, Langeronia soudanense, Leptosphaeria senegalensis, Lichtheimia corymbifera, Lobmyces loboi., Loboa loboi, Lobomycosis, Madurella spp., Malassezia furfur, Micrococcus pelletieri, Microsporum spp, Monilia spp., Mucor spp., Mycobacterium tuberculosis, Nannizzia spp., Neotestudina rosatii, Nocardia spp., Oidium albicans, Oospora lactis, Paracoccidioides brasiliensis, Petriellidium boydii, Phialophora spp., Piedraia hortae, Pityrosporum furfur, Pneumocystis jirovecii (or Pneumocystis carinii), Pullularia gougerotii, Pyrenochaeta romeroi, Rhinosporidium seeberi, Sabouraudites (Microsporum), Sartorya fumigate, Sepedonium, Sporotrichum spp., Stachybotrys, Stachybotrys chartarum, Streptomyce spp., Tinea spp., Torula spp, Trichophyton spp, Trichosporon spp, and Zopfia rosatii.

Additionally, bacterial taxon contemplated by the disclosure include any of the bacterial taxon provided in List 3 or List 4. It will be apparent to one of ordinary skill in the art that List 3 and List 4 are not exhaustive and are provided as exemplary lists.

List 3: Bacterial Genera:

Heliobacter, Aerobacter, Rhizobium, Agrobacterium, Bacillus, Clostridium, Pseudomonas, Xanthomonas, Nitrobacteriaceae, Nitrobacter, Nitrosomonas, Thiobacillus, Spirillum, Vibrio, Bacteroides, Corynebacterium, Listeria, Escherichia, Klebsiella, Salmonella, Serratia, Shigella, Erwinia, Rickettsia, Chlamydia, Mycoplasma, Actinomyces, Streptomyces, Mycobacterium, Polyangium, Micrococcus, Staphylococcus, Lactobacillus, Diplococcus, Streptococcus, and Campylobacter.

List 4: Bacterial Species:

Actinomyces israelii, Bacillus anthracis, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, and Yersinia pseudotuberculosis.

Pathogens include viruses, fungi, helminths, protozoa, malarial parasites, Plasmodium parasites, Toxoplasma parasites, Schistosoma parasites, and the like. “Helminths” include roundworms, heartworms, and phytophagous nematodes (Nematoda), flukes (Tematoda), Acanthocephala, and tapeworms (Cestoda). Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, Plasmodium vivax, Trypanosoma cruzi and Toxoplasma gondii. Fungal pathogens include, but are not limited to: Cryptococcus neoformans, Histoplasma capsulatum, Coccidioides immitis, Blastomyces dermatitidis, Chlamydia trachomatis, and Candida albicans. Pathogenic viruses include, e.g., immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogenic viruses can include DNA viruses such as: a papovavirus (e.g., human papillomavirus (HPV), polyomavirus); a hepadnavirus (e.g., Hepatitis B Virus (HBV)); a herpesvirus (e.g., herpes simplex virus (HSV), varicella zoster virus (VZV), epstein-barr virus (EBV), cytomegalovirus (CMV), herpes lymphotropic virus, Pityriasis Rosea, kaposi's sarcoma-associated herpesvirus); an adenovirus (e.g., atadenovirus, aviadenovirus, ichtadenovirus, mastadenovirus, siadenovirus); a poxvirus (e.g., smallpox, vaccinia virus, cowpox virus, monkeypox virus, orf virus, pseudocowpox, bovine papular stomatitis virus; tanapox virus, yaba monkey tumor virus; molluscum contagiosum virus (MCV)); a parvovirus (e.g., adeno-associated virus (AAV), Parvovirus B19, human bocavirus, bufavirus, human parv4 G1); Geminiviridae; Nanoviridae; Phycodnaviridae; and the like. Pathogens can include, e.g., DNA viruses [e.g.: a papovavirus (e.g., human papillomavirus (HPV), polyomavirus); a hepadnavirus (e.g., Hepatitis B Virus (HBV)); a herpesvirus (e.g., herpes simplex virus (HSV), varicella zoster virus (VZV), Epstein-barr virus (EBV), cytomegalovirus (CMV), herpes lymphotropic virus, Pityriasis Rosea, kaposi's sarcoma-associated herpesvirus); an adenovirus (e.g., atadenovirus, aviadenovirus, ichtadenovirus, mastadenovirus, siadenovirus); a poxvirus (e.g., smallpox, vaccinia virus, cowpox virus, monkeypox virus, orf virus, pseudocowpox, bovine papular stomatitis virus; tanapox virus, yaba monkey tumor virus; molluscum contagiosum virus (MCV)); a parvovirus (e.g., adeno-associated virus (AAV), Parvovirus B19, human bocavirus, bufavirus, human parv4 G1); Geminiviridae; Nanoviridae; Phycodnaviridae; and the like], Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae.

The terms “peptide” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

As used herein, the term “primer” refers to oligomeric compounds, primarily to oligonucleotides containing naturally occurring nucleotides such as adenine, guanine, cytosine, thymine and/or uracil, but may also include modified oligonucleotides (e.g., modified nucleotides, nucleosides, synthetic nucleotides having modified base moieties and/or modified sugar moieties (See, Protocols for Oligonucleotide Conjugates, Methods in Molecular Biology, Vol 26, (Sudhir Agrawal, Ed., Humana Press, Totowa, N.J., (1994)); and Oligonucleotides and Analogues, A Practical Approach (Fritz Eckstein, Ed., IRL Press, Oxford University Press, Oxford) that are able to prime polynucleotde (e.g., DNA) synthesis by an enzyme, typically in a template-dependent manner, i.e., the 3′ end of the primer provides a free 3′-OH group to which further nucleotides are attached by the enzyme (e.g., DNA polymerase or reverse transcriptase) establishing a 3′ to 5′ phosphodiester linkage whereby nucleoside triphosphates are used and pyrophosphate is released. Oligonucleotides can be prepared by any suitable method, including, for example, cloning and restriction of appropriate sequences and direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Lett. 22:1859-1862; and the solid support method of U.S. Pat. No. 4,458,066. A review of synthesis methods is provided in Goodchild, 1990, Bioconjugate Chemistry 1(3):165-187.

A primer is typically a single-stranded deoxyribonucleic acid. The appropriate length of a primer depends on the intended use of the primer but typically ranges from 6 to 50 nucleotides. Short primer molecules (e.g., having a length within a range of 11-17 nucleotides) generally require cooler temperatures to form sufficiently stable hybrid complexes with a template (or target) nucleic acid.

As used herein, a “reagent” refers broadly to any agent used in a reaction, other than the analyte (e.g., nucleic acid molecule being analyzed). Illustrative reagents for a nucleic acid amplification reaction or sequencing assay include, but are not limited to, buffer, metal ions, polymerase, reverse transcriptase, primers, probes, template nucleic acid, nucleotides, labels, dyes, nucleases, adapters, oligo-coated beads, microparticles or droplets, and the like. Generally, reagents for enzymatic reactions include, for example, substrates, cofactors, buffers, metal ions, inhibitors, and/or activators.

The term “sample” is used herein to mean any sample that includes DNA (e.g., in order to determine whether a target DNA is present among a population of DNAs). As noted above, nucleic acids include dsDNAs; or in the case of a ssDNA, a dsDNA prepared from a ssDNA, e.g., by second strand synthesis using the ssDNA as a template; or in the case of an RNA, a dsDNA prepared from an RNA, e.g., by reverse transcription using the RNA as a template to generate a cDNA, and second strand synthesis using the cDNA as a template. The sample can be derived from any source, e.g., the sample can be a synthetic combination of purified DNAs; the sample can be a cell lysate, a DNA-enriched cell lysate, or DNAs isolated and/or purified from a cell lysate, or DNAs isolated and/or purified from an environmental sample. The sample can be from a patient (e.g., for the purpose of diagnosis). The sample can be from permeabilized cells. The sample can be from crosslinked cells. The sample can be in tissue sections. The sample can be from tissues prepared by crosslinking followed by delipidation and adjustment to make a uniform refractive index. Examples of tissue preparation by crosslinking followed by delipidation and adjustment to make a uniform refractive index have been described in, for example, Shah et al., Development (2016) 143, 2862-2867 doi:10.1242/dev.138560.

As used herein, the term “sample” refers to a sample collected from a subject including, but not limited to, human and non-human animal subjects, that may be affected by or are suspected of infection by a pathogen (e.g., an infectious bacterium, protozoa, prion, fungi, algae, parasite or other microbe). The term also includes samples collected from the environment including, but not limited to, surface samples, water samples, soil samples and the like. A sample includes but is not limited to, a cell, cell lysate, isolated DNA, isolated RNA, tissue section, tissue biopsy, liquid biopsy, blood, or other biological fluid (e.g., cerebrospinal fluid) obtained from a subject. A sample includes blood samples (e.g., whole peripheral blood, serum or plasma), tissue samples (e.g., fresh, frozen or Fixed Formalin Paraffin Embedded (FFPE) samples, biopsy samples (e.g., fine needle aspirates (FNAs)), excretions and secretions such as, saliva, sputum, urine, stool, plasma/serum, breast milk, sperm, semen, vaginal secretions, sweat, mucus, bile, and oral and genital mucosal swabs. The sample can include a clinical sample (e.g., a patient sample) for the purpose of diagnosis, detection, epidemiology, treatment, disease monitoring, and the like. In some instances, the sample comprises isolated RNA and/or DNA from a mammal (e.g., pig, cow, goat, sheep, rodent, rat, mouse, dog, cat, non-human primate or human). A tissue sample typically includes one or more cells obtained from a tissue of the subject or cells derived from a tissue obtained from the subject (e.g., cells in tissue culture). It will be apparent to one of ordinary skill in the art that a tissue sample can include cells obtained from a somatic tissue (e.g., liver, kidney, spleen, gall bladder, stomach, bladder, uterus, intestines, pancreas, colon, lung, heart, brain, muscle, bone, pharynx and larynx).

A “sample” can include a target DNA and a plurality of non-target DNAs. In some cases, the target DNA is present in the sample at one copy per 10 non-target DNAs, one copy per 20 non-target DNAs, one copy per 25 non-target DNAs, one copy per 50 non-target DNAs, one copy per 100 non-target DNAs, one copy per 500 non-target DNAs, one copy per 10³ non-target DNAs, one copy per 5×10³ non-target DNAs, one copy per 10⁴ non-target DNAs, one copy per 5×10⁴ non-target DNAs, one copy per 10⁵ non-target DNAs, one copy per 5×10⁵ non-target DNAs, one copy per 10⁶ non-target DNAs, or less than one copy per 10⁶ non-target DNAs. In some cases, the target DNA is present in the sample at from one copy per 10 non-target DNAs to 1 copy per 20 non-target DNAs, from 1 copy per 20 non-target DNAs to 1 copy per 50 non-target DNAs, from 1 copy per 50 non-target DNAs to 1 copy per 100 non-target DNAs, from 1 copy per 100 non-target DNAs to 1 copy per 500 non-target DNAs, from 1 copy per 500 non-target DNAs to 1 copy per 10³ non-target DNAs, from 1 copy per 10³ non-target DNAs to 1 copy per 5×10³ non-target DNAs, from 1 copy per 5×10³ non-target DNAs to 1 copy per 10⁴ non-target DNAs, from 1 copy per 10⁴ non-target DNAs to 1 copy per 10⁵ non-target DNAs, from 1 copy per 10⁵ non-target DNAs to 1 copy per 10⁶ non-target DNAs, or from 1 copy per 10⁶ non-target DNAs to 1 copy per 10⁷ non-target DNAs.

Suitable samples include, but are not limited to, saliva, blood, serum, plasma, urine, aspirate, water, swabs of surfaces, food, and biopsy samples. Thus, the term “sample” with respect to a patient encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as cancer cells. The definition also includes sample that have been enriched for particular types of molecules, e.g., DNAs. The term “sample” encompasses biological samples such as a clinical sample such as blood, plasma, serum, aspirate, cerebrospinal fluid (CSF), a bronchoalveolar lavage sample, or sputum; and also includes tissue obtained by surgical resection, tissue obtained by biopsy, cells in culture, cell supernatants, cell lysates, tissue samples, organs, bone marrow, and the like. A “biological sample” includes biological fluids derived therefrom (e.g., cancerous cell, infected cell, etc.), e.g., a sample comprising DNAs that is obtained from such cells (e.g., a cell lysate or other cell extract comprising DNAs).

A sample can comprise, or can be obtained from, any of a variety of cells, tissues, organs, or acellular fluids. Suitable sample sources include eukaryotic cells, bacterial cells, and archaeal cells. Suitable sample sources include single-celled organisms and multi-cellular organisms. Suitable sample sources include single-cell eukaryotic organisms; a plant or a plant cell; an algal cell, e.g., Botryococcus braunii, Chlamydomonas reinhardtii, Nannochloropsis gaditana, Chlorella pyrenoidosa, Sargassum patens, C. agardh, and the like; a fungal cell (e.g., a yeast cell); an animal cell, tissue, or organ; a cell, tissue, or organ from an invertebrate animal (e.g. fruit fly, cnidarian, echinoderm, nematode, an insect, an arachnid, etc.); a cell, tissue, fluid, or organ from a vertebrate animal (e.g., fish, amphibian, reptile, bird, mammal); a cell, tissue, fluid, or organ from a mammal (e.g., a human; a non-human primate; an ungulate; a feline; a bovine; an ovine; a caprine; etc.). Suitable sample sources include nematodes, protozoans, and the like. Suitable sample sources include parasites such as helminths, malarial parasites, etc.

Suitable sample sources include a cell, tissue, or organism of any of the six kingdoms, e.g., Bacteria (e.g., Eubacteria); Archaebacteria; Protista; Fungi; Plantae; and Animalia. Suitable sample sources include plant-like members of the kingdom Protista, including, but not limited to, algae (e.g., green algae, red algae, glaucophytes, cyanobacteria); fungus-like members of Protista, e.g., slime molds, water molds, etc.; animal-like members of Protista, e.g., flagellates (e.g., Euglena), amoeboids (e.g., amoeba), sporozoans (e.g, Apicomplexa, Myxozoa, Microsporidia), and ciliates (e.g., Paramecium). Suitable sample sources include include members of the kingdom Fungi, including, but not limited to, members of any of the phyla: Basidiomycota (club fungi; e.g., members of Agaricus, Amanita, Boletus, Cantherellus, etc.); Ascomycota (sac fungi, including, e.g., Saccharomyces); Mycophycophyta (lichens); Zygomycota (conjugation fungi); and Deuteromycota. Suitable sample sources include include members of the kingdom Plantae, including, but not limited to, members of any of the following divisions: Bryophyta (e.g., mosses), Anthocerotophyta (e.g., hornworts), Hepaticophyta (e.g., liverworts), Lycophyta (e.g., club mosses), Sphenophyta (e.g., horsetails), Psilophyta (e.g., whisk ferns), Ophioglossophyta, Pterophyta (e.g., ferns), Cycadophyta, Gingkophyta, Pinophyta, Gnetophyta, and Magnoliophyta (e.g., flowering plants). Suitable sample sources include include members of the kingdom Animalia, including, but not limited to, members of any of the following phyla: Porifera (sponges); Placozoa; Orthonectida (parasites of marine invertebrates); Rhombozoa; Cnidaria (corals, anemones, jellyfish, sea pens, sea pansies, sea wasps); Ctenophora (comb jellies); Platyhelminthes (flatworms); Nemertina (ribbon worms); Ngathostomulida (jawed worms)p Gastrotricha; Rotifera; Priapulida; Kinorhyncha; Loricifera; Acanthocephala; Entoprocta; Nemotoda; Nematomorpha; Cycliophora; Mollusca (mollusks); Sipuncula (peanut worms); Annelida (segmented worms); Tardigrada (water bears); Onychophora (velvet worms); Arthropoda (including the subphyla: Chelicerata, Myriapoda, Hexapoda, and Crustacea, where the Chelicerata include, e.g., arachnids, Merostomata, and Pycnogonida, where the Myriapoda include, e.g., Chilopoda (centipedes), Diplopoda (millipedes), Paropoda, and Symphyla, where the Hexapoda include insects, and where the Crustacea include shrimp, krill, barnacles, etc.; Phoronida; Ectoprocta (moss animals); Brachiopoda; Echinodermata (e.g. starfish, sea daisies, feather stars, sea urchins, sea cucumbers, brittle stars, brittle baskets, etc.); Chaetognatha (arrow worms); Hemichordata (acorn worms); and Chordata. Suitable members of Chordata include any member of the following subphyla: Urochordata (sea squirts; including Ascidiacea, Thaliacea, and Larvacea); Cephalochordata (lancelets); Myxini (hagfish); and Vertebrata, where members of Vertebrata include, e.g., members of Petromyzontida (lampreys), Chondrichthyces (cartilaginous fish), Actinopterygii (ray-finned fish), Actinista (coelocanths), Dipnoi (lungfish), Reptilia (reptiles, e.g., snakes, alligators, crocodiles, lizards, etc.), Ayes (birds); and Mammalian (mammals). Suitable plants include any monocotyledon and any dicotyledon.

Suitable sources of a sample include cells, fluid, tissue, or organ taken from an organism; from a particular cell or group of cells isolated from an organism; etc. For example, where the organism is a plant, suitable sources include xylem, the phloem, the cambium layer, leaves, roots, etc. Where the organism is an animal, suitable sources include particular tissues (e.g., lung, liver, heart, kidney, brain, spleen, skin, fetal tissue, etc.), or a particular celltype (e.g., neuronal cells, epithelial cells, endothelial cells, astrocytes, hepatocytes, cardiac cells, macrophages, glial cells, islet cells, T lymphocytes, B lymphocytes, etc.).

In some cases, the source of the sample is a (or is suspected of being a diseased cell, fluid, tissue, or organ. In some cases, the source of the sample is a normal (non-diseased) cell, fluid, tissue, or organ. In some cases, the source of the sample is a (or is suspected of being a pathogen-infected cell, tissue, or organ. For example, the source of a sample can be an individual who may or may not be infected—and the sample could be any biological sample (e.g., blood, saliva, biopsy, plasma, serum, bronchoalveolar lavage, sputum, a fecal sample, cerebrospinal fluid, a fine needle aspirate, a swab sample (e.g., a buccal swab, a cervical swab, a nasal swab), interstitial fluid, synovial fluid, nasal discharge, tears, buffy coat, a mucous membrane sample, an epithelial cell sample (e.g., epithelial cell scraping), etc.) collected from the individual. In some cases, the sample is a cell-free liquid sample. In some cases, the sample is a liquid sample that can comprise cells.

In one embodiment, the target nucleic acids is 16S ribosomal RNA (rRNA) where the target nucleic acid is a nucleic acid of a bacterium, 18S and 28S regions of fungi, 18S of Babesia, and rpoB and hsp65 of mycobacteria etc. In a further embodiment, the target nucleic acids is first amplified with a universal primer as set forth in Table 1. In a further embodiment, the amplified product resulting from the use of one or more universal primers of Table 1 are then contacted with specific sequences for a subset or specific species of oganism using a CRISPR/Cas system of the disclosure.

In some cases, e.g., where the target nucleic acid is a nucleic acid of a parasite (e.g., Acanthamoeba, Angiostrongylus, Ascaris, Babesia, Balamuthia, Blastocytis, Brugia, Cyclospora, Echinococcus, Entamoeba, Fasciola, Giardia, Leishmania, Loa Loa, Naegleria, Schistosoma, Strongyloides, Taenia, Toxoplasma, Trichinella, Trypanosoma, Plasmodium, and the like), a target nucleic acid can be an 18S rRNA.

A sample includes a nucleic acid. As noted above, nucleic acids include: i) dsDNAs; ii) ssDNA, where a dsDNA can be prepared from a ssDNA, e.g., by second strand synthesis using the ssDNA as a template; and iii) RNA, where a dsDNA can be prepared from an RNA, e.g., by reverse transcription using the RNA as a template to generate a cDNA, and second strand synthesis using the cDNA as a template. For simplicity, the discussion herein refers to “DNA” or “DNAs”; however, the nucleic acid being detected can be a dsDNA that is prepared from a ssDNA or from an RNA.

A subject sample includes nucleic acid (e.g., a plurality of nucleic acids). The term “plurality” is used herein to mean two or more. In some cases, a sample includes two or more (e.g., 3 or more, 5 or more, 10 or more, 20 or more, 50 or more, 100 or more, 500 or more, 1,000 or more, or 5,000 or more) nucleic acids (e.g., dsDNAs; or in the case of a ssDNA, a dsDNA prepared from a ssDNA, e.g., by second strand synthesis using the ssDNA as a template; or in the case of an RNA, a dsDNA prepared from an RNA, e.g., by reverse transcription using the RNA as a template to generate a cDNA, and second strand synthesis using the cDNA as a template). In some cases, the sample includes 5 or more DNAs (e.g., 10 or more, 20 or more, 50 or more, 100 or more, 500 or more, 1,000 or more, or 5,000 or more DNAs) that differ from one another in sequence. In some cases, the sample includes 10 or more, 20 or more, 50 or more, 100 or more, 500 or more, 10³ or more, 5×10³ or more, 10⁴ or more, 5×10⁴ or more, 10⁵ or more, 5×10⁵ or more, 10⁶ or more 5×10⁶ or more, or 10⁷ or more, DNAs. In some cases, the sample comprises from 10 to 20, from 20 to 50, from 50 to 100, from 100 to 500, from 500 to 10³ from 10³ to 5×10³, from 5×10³ to 10⁴, from 10⁴ to 5×10⁴, from 5×10⁴ to 10⁵, from 10⁵ to 5×10⁵, from 5×10⁵ to 10⁶, from 10⁶ to 5×10⁶, or from 5×10⁶ to 10⁷, or more than 10⁷, DNAs. In some cases, the sample comprises from 5 to 10⁷ DNAs (e.g., that differ from one another in sequence) (e.g., from 5 to 10⁶, from 5 to 10⁵, from 5 to 50,000, from 5 to 30,000, from 10 to 10⁶, from 10 to 10⁵, from 10 to 50,000, from 10 to 30,000, from 20 to 10⁶, from 20 to 10⁵, from 20 to 50,000, or from 20 to 30,000 DNAs). In some cases, the sample includes 20 or more DNAs that differ from one another in sequence. In some cases, the sample includes DNAs from a cell lysate (e.g., a eukaryotic cell lysate, a mammalian cell lysate, a human cell lysate, a prokaryotic cell lysate, a plant cell lysate, and the like). For example, in some cases, the sample includes DNA from a cell such as a eukaryotic cell, e.g., a mammalian cell such as a human cell.

As used herein, the term “subject” refers to any member of the class animals, including, without limitation, humans and other primates, including non-human primates such as rhesus macaques, chimpanzees and other monkey and ape species; farm animals, such as cattle, sheep, pigs, goats and horses; domestic mammals, such as dogs and cats; laboratory animals, including rabbits, mice, rats and guinea pigs; birds and other reptiles, including domestic, wild, and game birds, such as chickens, turkeys, geese, ducks, lizards, alligators, and snakes; amphibians, including frogs, toads, salamanders, and newts; fish, such as salmon, and tilapia; and insects. The term does not denote a particular age or gender. Thus, adult, young, and newborn subjects are intended to be included as well as male and female subjects. In most instances, the subject is a host to the pathogen and the pathogen may rely on its ability to infect the host, for example the production of toxins, to enter cells and tissues within the host, and acquire host nutrients to maintain infectiousness. The term includes subjects who are experiencing or have experienced illness or disease associated with a particular taxon of pathogenic microorganisms or subjects who are infected (or suspected of being infected) with a particular taxon of pathogen but are not experiencing or demonstrating symptoms of illness or disease associated with the pathogen.

As used herein, a “target” refers to a molecule of interest to be detected in a sample. In some embodiments, the target is a nucleic acid molecule. In a one embodiment, the target is a target DNA, target RNA or target nucleic acid from a pathogen. In some embodiments, the target is a polynucleotide, such as dsDNA or ssDNA; RNA, such as ssRNA or dsRNA, or a DNA-RNA hybrid. In some embodiments, two or more target molecules are detected in a single sample. In some embodiments, the two or more target molecules may be related to each other (e.g., nucleic acids from the same taxon, genus or species of pathogens). In another embodiment, a first target molecule is from a first taxon of pathogenic microorganisms and a second target molecule is from a second taxon of pathogens. In some embodiments, the target nucleic can be from the host subject and not a pathogen.

In some instances, a target sequence or target nucleic acid molecule refers to a region, subsequence, or complete nucleic acid molecule which is to be amplified (e.g., RNA to cDNA, or amplification of DNA) or detected using the method, kits and compositions disclosed herein. Accordingly, amplification of one or more target sequences can include detection of one or more pathogenic microorganisms in a single sample, such as but not limited to, the detection and/or identification of a co-infection in the sample. For example, a clinical sample from a subject (e.g., a serum or urine sample from a human subject) can be evaluated for the presence (or absence) of an amplified target sequence present in the genome of a microorganism. Identification of two target sequences from distinct taxa from different domains (e.g., bacterial and fungal domains) would be indicative that the subject is infected by both pathogenic microorganisms (e.g., a fungal pathogen and a bacterial pathogen). Identification of the target sequence in the sample can be useful for the modulation of the form, dosage, or regime of treatment for the subject affected by the pathogen.

As used herein, the term “thermostable polymerase” refers to a polymerase enzyme that is heat stable, i.e., the enzyme catalyzes the formation of a primer extension product complementary to a template nucleic acid, and is not irreversibly denatured when subjected to elevated temperatures for the time needed to effect denaturation of double-stranded template nucleic acids (e.g., between 95° C.-99° C.). Thermostable polymerases have been isolated from Thermus flavus, T. ruber, T. thermophilus, T. aquaticus, T. lacteus, T. rubens, Bacillus stearothermophilus, and Methanothermus fervidus. Additionally, polymerases that are not thermostable can be employed in the PCR assays disclosed herein, for example by replenishing the polymerase between synthesis/extension and denaturation steps as it becomes denatured. Any polymerase or thermostable polymerase known in the art is suitable for use in the method disclosed herein.

The disclosure provides methods for characterizing a target DNA that is present in a sample. The methods involve contacting the sample with one or a plurality of universal primers to amplify a taxon of a target microorganism(s) in the sample. The method further includes contacting the sample with a type V CRISPR/Cas effector protein and one or more guide RNAs. The type V CRISPR/Cas proteins, e.g., Cas12 proteins such as Cpf1 (Cas12a) and C2c1 (Cas12b) can promiscuously cleave non-targeted single stranded DNA (ssDNA) once activated by detection of a target DNA (double or single stranded). Once a type V CRISPR/Cas effector protein (e.g., a Cas12 protein such as Cas12a, Cas12b, Cas12c, Cas12d, Cas12e) is activated by a guide RNA, which occurs when the guide RNA hybridizes to a target sequence of a target DNA (i.e., the sample includes the targeted DNA), the protein becomes a nuclease that promiscuously cleaves ssDNAs (i.e., the nuclease cleaves non-target ssDNAs, i.e., ssDNAs to which the guide sequence of the guide RNA does not hybridize). Thus, when the target DNA is present in the sample (e.g., in some cases above a threshold amount), the result is cleavage of ssDNAs in the sample, which can be detected using any convenient detection method (e.g., using a labeled single stranded detector DNA) (see, e.g., U.S. Pat. Publ. No. 20190241954, which is incorporated herein by reference for all purposes). In some embodiments, the guide RNA(s) are complementary to a specific taxon or microorganism.

The sample can be subjected to one or more amplification steps prior to the contacting step, with universal primers that provide for amplification of nucleic acids of, e.g., specific pathogens, categories of pathogens, two or more different pathogens, or two or more different categories of pathogens. The pre-amplified sample can also be contacted with a ssDNA reporter molecule that provides a readout when the type V CRISPR/Cas effector contacts the target DNA. The disclosure provides a kit comprising components for carrying out a method of the disclosure. A kit or method of the disclosure finds use in a wide variety of areas, including, e.g., infectious disease identification, and the like.

In some cases, the primers are universal primers, as described in the Examples. In some cases, a method of the disclosure comprises use of metagenomic sequencing with universal primers.

As noted above, a sample comprising a target DNA can be subjected to one or more nucleic acid amplification steps before the contacting step. The sample can be subjected to one or more amplification steps prior to the contacting step, with primers that provide for amplification of nucleic acids of, e.g., a population of organisms belonging to a particular taxa.

A sample comprising a target DNA can be amplified using a method comprising contacting the sample with one or more pairs of nucleic acid primers. For example, in some cases, the sample is contacted with a single pair of nucleic acid primers (also referred to herein as “oligonucleotide primers” or, simply, “primers”). In some cases, the sample is contacted with two or more different pairs of primers; e.g., the sample is contacted with 2, 3, 4, 5, 6, 7, 8, 9, or 10, or more than 10 (e.g., from 10 to 15, from 15 to 20, from 20 to 25, or from 25 to 30), different pairs of primers. The term “different pairs of primers” refers to primer pairs that differ from one another in nucleotide sequence. For example, a first primer pair differs from a second primer pair in nucleotide sequence, where the first and second primer pairs are “different pairs of primers.”

In some cases, the two or more different primer pairs provide for amplification of DNA from two or more different pathogens. In some cases, the two or more different primer pairs provide for amplification of DNA from two or more (e.g., 2, 3, 4, 5, 6, 7, 8, 9, 10, or more than 10 (e.g., from 10 to 15, from 15 to 20, from 20 to 25, or from 25 to 30)) different pathogens. In some cases, the two or more pathogens are bacterial pathogens. In some cases, the two or more pathogens are helminths. In some cases, the two or more pathogens are protozoa. In some cases, the two or more pathogens are fungal pathogens.

In some case, the two or more different primer pairs provide for amplification of DNA from two or more categories of pathogens. For example, in some cases, the two or more different primer pairs provide for amplification of DNA of two or more different tick-borne pathogens. As another example, in some cases, the two or more different primer pairs provide for amplification of DNA of two or more different mosquito-borne pathogens. As another example, in some cases, the two or more different primer pairs provide for amplification of DNA of two or more antibiotic-resistant pathogens.

For example, forward and reverse primers that can be used in connection with detection of a population of a particular taxa. The disclosure provides a number of universal primers that comprise, consist essential of, or consist of the sequences as set forth in Table 1 for the detection of various microbial populations as set forth in Table 1. The primers of Table 1 can comprise 1-5 additional nucleotides at either end or 1-5 fewer nucleotides in some instances.

TABLE 1
UNIVERSAL BACTERIAL PRIMERS

| | |
|---|---|
| 302F-universal-Tm54 | CACACTGGRACTGAGAYACGG (SEQ ID NO: 1) |
| 333F-universal-Tm55-2X | ACTCCTACGGGAGGCWGCA (SEQ ID NO: 2) |
| 528F-universal-Tm57-4X | GTGCCAGCAGYYGCGGTA(SEQ ID NO: 3) |
| 802R-universal-Tm52-8X | GAYTACYRGGGTATCTAATCC(SEQ ID NO: 4) |
| 897R-universal-Tm53-6X | CCCCGTCAATTHMTTTGAGTTT(SEQ ID NO: 5) |
| 1072R-universal-Tm55-2X | CGTTRCGGGACTTAACCCAACA(SEQ ID NO: 6) |
| 1166R-Tm53-4X | TCRTCCYCACCTTCCTCC(SEQ ID NO: 7) |
| 1380R-Tm52-8X | YCCGRGAACGTATTCACSG(SEQ ID NO: 8) |

BABESIA PRIMERS

| | |
|---|---|
| 18S-304F-2-fold-Tm50.7 | GGTATTGGCCTACCGRG(SEQ ID NO: 9) |
| 18S-413F-0-fold-Tm51.1 | TACCCAATCCTGACACAGG(SEQID NO: 10) |
| 18S-877R-2-fold-Tm53.4 | GCTTTCGCAGTRGTTCGTCTT(SEQ ID NO: 11) |
| 18S-931R-0-fold-Tm51.8 | CGTCTTCGATCCCCTAACTT(SEQ ID NO: 12) |
| 18S-1018F-0-fold-Tm51.1 | GACTCCTTCAGCACCTTGA(SEQ ID NO: 13) |
| 18S-1619R-0-fold-Tm50.5 | CGAATAATTCACCGGATCACT(SEQ ID NO: 14) |
| 18S-1679R-0-fold-Tm51.7 | AGTTTTGTGAACCTTATCACTTAAAG(SEQ ID NO: 15) |

MYCOBACTERIAL PRIMERS

| | |
|---|---|
| Mycobacterium-rpoB-259F-4XTm47.1-51.9 | CACGGCAAYAAGGGYGT(SEQ ID NO: 16) |
| Mycobacterium-rpoB-274F-6XTm45.8-50.3 | GTBATCGGCAAGATYCTC(SEQ ID NO: 17) |
| Mycobacterium-rpoB-697R-1XTm51.9 | TCACCGGGTACGGGAAC(SEQ ID NO: 18) |
| Mycobacterium-rpoB-755R-4XTm47.1-49.5 | GCGTGRATCTTGTCRTC(SEQ ID NO: 19) |
| Mycobacterium-hsp65-322F-2XTm = 52.6 | TACGAGAAGATCGGCGCY(SEQ ID NO: 20) |
| Mycobacterium-hsp65-282F-1XTm = 52.6 | GGTGTGTCCATCGCCAAG(SEQ ID NO: 21) |
| Mycobacterium-hsp65-650R-3XTm = 51.9 | CTCGTTGCCVACCTTGTC(SEQ ID NO: 22) |
| Mycobacterium-hsp65-670R-2XTm = 52.6 | CTCGACGGTGATGACRCC(SEQ ID NO: 23) |

FUNGAL PRIMERS

| | |
|---|---|
| Fungal-18S-SSU-forward1-4X | GTACACACKCCYGTCG(SEQ ID NO: 24) |
| Fungal-18S-SSU-forward2-6X | TGYAATTDTTGCTCTTCAACGAG(SEQ ID NO: 25) |
| Fungal-296R-4X | GCTSCGTTCTTCATCGATSC(SEQ ID NO: 26) |
| Fungal-350R-4X | GTTCAAGAYTCRATGATTCAC(SEQ ID NO: 27) |
| Fungal-296R-Pneumocystis | GCCACGTTCTTCATCGACGC(SEQID NO: 28) |
| Fungal-350R-Pneumocystis | GTTCAAAAATTCGATGATTCAC(SEQID NO: 29) |

R = A or G; Y = C or T; W = A or T; K = G or T; M = A or C; B = C or G or T; H = A or C or T; V = A or C or G; S = G or C; D = A, G or T
Tm = melting temperature in Celsius
F = forward primer
R = reverse primer
1X, 2X, 4X, 6X, 8X refer to degeneracy of the primer and input concentration must account for the degeneracy (i.e., 2X degenerate primer is added at 2X the concentration of a 1X primer (non-degenerate) etc.)

It should be recognized that any of the sequences of Table 1 can have “T” replaced by “U” for RNA.

The universal primers of Table 1 are useful for identification and/or amplification of nucleic acid associated with the microbial class for which they are directed. For example, if one of skill in the art wanted to determine the presence of a fungal organism in a sample, primers having SEQ ID NOs: 24-29 would be used to identify and/or amplify nucleic acids from the sample. Thus, determining the presence of a fungal organism being present in the sample. Further sequencing could be used to determine a specific taxa (e.g. species) of the fungal organism, thus identifying, for example, an infection by a fungal organism.

In one embodiment, the assay comprises at least one primer selected from any of SEQ ID NOs: 1-8 for bacterial detection; SEQ ID NOs: 9-15 for Babesia detection; SEQ ID NOs: 16-23 for mycobacterium detection and SEQ ID NOs: 24-29 for fungi detection. It will be readily apparent that where co-infection or multiple organism may be present any combination of SEQ ID NOs: 1-29 may be used.

Various amplification methods and components will be known to one of ordinary skill in the art and any convenient method can be used (see, e.g., Zanoli and Spoto, Biosensors (Basel). 2013 March; 3(1): 18-43; Gill and Ghaemi, Nucleosides, Nucleotides, and Nucleic Acids, 2008, 27: 224-243; Craw and Balachandrana, Lab Chip, 2012, 12, 2469-2486; which are herein incorporated by reference in their entirety). Nucleic acid amplification can comprise polymerase chain reaction (PCR), reverse transcription PCR (RT-PCR), quantitative PCR (qPCR), reverse transcription qPCR (RT-qPCR), nested PCR, multiplex PCR, asymmetric PCR, touchdown PCR, random primer PCR, hemi-nested PCR, polymerase cycling assembly (PCA), colony PCR, ligase chain reaction (LCR), digital PCR, methylation specific-PCR (MSP),co-amplification at lower denaturation temperature-PCR (COLD-PCR), allele-specific PCR, intersequence-specific PCR (ISS-PCR), whole genome amplification (WGA), inverse PCR, and thermal asymmetric interlaced PCR (TAIL-PCR).

In some cases, the amplification is isothermal amplification. The term “isothermal amplification” indicates a method of nucleic acid (e.g., DNA) amplification (e.g., using enzymatic chain reaction) that can use a single temperature incubation thereby obviating the need for a thermal cycler. Isothermal amplification is a form of nucleic acid amplification which does not rely on the thermal denaturation of the target nucleic acid during the amplification reaction and hence may not require multiple rapid changes in temperature. Isothermal nucleic acid amplification methods can therefore be carried out inside or outside of a laboratory environment. By combining with a reverse transcription step, these amplification methods can be used to isothermally amplify RNA.

Examples of isothermal amplification methods include, but are not limited to, loop-mediated isothermal amplification (LAMP), helicase-dependent Amplification (HDA), recombinase polymerase amplification (RPA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification (SPIA), signal mediated amplification of RNA technology (SMART), self-sustained sequence replication (3SR), genome exponential amplification reaction (GEAR) and isothermal multiple displacement amplification (IMDA).

In some cases, the amplification is recombinase polymerase amplification (RPA) (see, e.g., U.S. Pat. Nos. 8,030,000; 8,426,134; 8,945,845; 9,309,502; and 9,663,820, which are hereby incorporated by reference in their entirety). Recombinase polymerase amplification (RPA) uses two opposing primers (much like PCR) and employs three enzymes—a recombinase, a single-stranded DNA-binding protein (SSB) and a strand-displacing polymerase. The recombinase pairs oligonucleotide primers with homologous sequence in duplex DNA, SSB binds to displaced strands of DNA to prevent the primers from being displaced, and the strand displacing polymerase begins DNA synthesis where the primer has bound to the target DNA. Adding a reverse transcriptase enzyme to an RPA reaction can facilitate detection RNA as well as DNA, without the need for a separate step to produce cDNA. One example of components for an RPA reaction is as follows (see, e.g., U.S. Pat. Nos. 8,030,000; 8,426,134; 8,945,845; 9,309,502; 9,663,820): 50 mM Tris pH 8.4, 80 mM Potassium actetate, 10 mM Magnesium acetate, 2 mM DTT, 5% PEG compound (Carbowax-20M), 3 mM ATP, 30 mM Phosphocreatine, 100 ng/μl creatine kinase, 420 ng/μl gp32, 140 ng/μl UvsX, 35 ng/μl UvsY, 2000M dNTPs, 300 nM each oligonucleotide, 35 ng/μl Bsu polymerase, and a nucleic acid-containing sample).

In a transcription-mediated amplification (TMA) method, an RNA polymerase is used to make RNA from a promoter engineered in the primer region; then a reverse transcriptase synthesizes cDNA from the primer. A third enzyme, e.g., Rnase H, can then be used to degrade the RNA target from cDNA without the heat-denatured step. This amplification technique is similar to Self-Sustained Sequence Replication (3SR) and Nucleic Acid Sequence Based Amplification (NASBA), but varies in the enzymes employed. As another example, helicase-dependent amplification (HDA) utilizes a thermostable helicase (Tte-UvrD) rather than heat to unwind dsDNA to create single-strands that are then available for hybridization and extension of primers by polymerase. As yet another example, a loop-mediated amplification (LAMP) method employs a thermostable polymerase with strand displacement capabilities and a set of four or more specific designed primers. Each primer is designed to have hairpin ends that, once displaced, snap into a hairpin to facilitate self-priming and further polymerase extension. In a LAMP reaction, though the reaction proceeds under isothermal conditions, an initial heat denaturation step is required for double-stranded targets. In addition, amplification yields a ladder pattern of various length products. As yet another example, a strand displacement amplification (SDA) combines the ability of a restriction endonuclease to nick the unmodified strand of its target DNA and an exonuclease-deficient DNA polymerase to extend the 3′ end at the nick and displace the downstream DNA strand.

In some cases, a target DNA present in a sample is generated from an RNA template. Any known method of generating DNA from an RNA template can be used. For example, a reverse transcriptase can be used to generate a target DNA from a target RNA.

In some instances, the sample is dehosted of non-target nucleic acid sequences. The disclosure also provides embodiments directed to dehosting a sample prior to the identification of a taxon or taxa of pathogenic microorganisms in a sample. Such dehosting techniques and compositions relate to the selective cleavage of non-microbial nucleic acids in a sample containing both pathogen-based nucleic acids and non-pathogen-based nucleic acids (e.g., nucleic acids from a subject), so that the sample becomes greatly enriched with microbial nucleic acids. Examples of dehosting methods include those described in Feehery et al., PLoS ONE 8:e76096 (2013); Sachse et al., Journal of Clinical Microbiology 47:1050-1057 (2009); Barnes et al., PLoS ONE 9(10):e109061 (2014); Leichty et al., Genetics 198(2):473-81 (2014)); Hasan et al., J Clin Microbiol 54(4):919-27 (2016); and Liu et al., PLoS ONE 11(1):e0146064 (2016). Additionally, commercial kits for carrying out dehosting are also available, including the NEBNext Microbiome DNA Enrichment™ Kit, the Molzym MolYsis Basic™ kit, and MICROBEEnrich™ Kit.

In some embodiments, the dehosting methods and compositions disclosed herein takes advantage of properties associated with non-pathogen-based nucleic acids, including methylation at CpG residues, and associations with DNA-binding proteins, such as histones. For example, in a particular embodiment the dehosting methods and compositions can utilizes a nucleic acid binding protein that selectively binds with non-pathogen-based nucleic acids (e.g., histones, restriction enzymes). In a further embodiment, the dehosting methods and compositions can comprise a recombinant protein that selectively binds with non-pathogen-based nucleic acids, and which also selectively degrades non-pathogen-based nucleic acids, i.e., the recombinant protein comprises both a nonmicrobial nucleic acid binding domain and a nuclease domain. In a particular embodiment, the nucleic acid binding protein is a histone. Histones are found in the nuclei of eukaryotic cells, and in certain Archaea, namely Thermoproteales and Euryarchaea, but not in bacteria or viruses. In a further embodiment, histone bound non-pathogen-based nucleic acids can then be removed from the sample by use of a substrate which comprises an affinity agent that selectively binds to a histone protein, i.e., a histone-binding domain. Examples of affinity agents that can bind to a histone protein include, but are not limited to, chromodomain, Tudor, Malignant Brain Tumor (MBT), plant homeodomain (PHD), bromodomain, SANT, YEATS, Proline-Tryptophan-Tryptophan-Proline (PWWP), Bromo Adjacent Homology (BAH), Ankryin repeat, WD40 repeat, ATRX-DNMT3A-DNMT3L (ADD), or zn-CW. In another embodiment, the histone-binding domain can include a domain which specifically binds to a histone from a protein such as HAT1, CBP/P300, PCAF/GCN5, TIP60, HB01 (ScESA1, SpMST1), ScSAS3, ScSAS2 (SpMST2), ScRTT109, SirT2 (ScSir2), SUV39H1, SUV39H2, G9a, ESET/SETDB1, EuHMTase/GLP, CLL8, SpClr4, MLL1, MLL2, MLL3, MLL4, MLL5, SET1A, SET1B, ASH1, Sc/Sp SET1, SET2 (Sc/Sp SET2) , NSD1, SYMD2, DOT1, Sc/Sp DOT1, Pr-SET 7/8, SUV4 20H1, SUV420H2, SpSet 9, EZH2, RIZ1, LSD1/BHC110, JHDM1a, JHDM1b, JHDM2a, JHDM2b, JMJD2A/JHDM3A, JMJD2B, JMJD2C/GASC1, JMJD2D, CARM1, PRMT4, PRMT5, Haspin, MSK1, MSK2, CKII, Mst1, Bmi/Ring1A, RNF20/RNF40, or ScFPR4, or a histone-binding fragment thereof.

In additional embodiment, the disclosure also provides for a nucleic acid binding protein or nucleic acid binding domain that selectively binds to DNA that comprises a methylated CpG. CG dinucleotide motifs (“CpG sites” or “CG sites”) are found in regions of DNA where a cytosine nucleotide is followed by a guanine nucleotide in the linear sequence of bases along its 5′ to 3′ direction. CpG islands (or CG islands) are regions with a high frequency of CpG sites. CpG is shorthand for 5′-C-phosphate-G-3′, that is, cytosine and guanine separated by one phosphate. Cytosines in CpG dinucleotides can be methylated to form 5-methylcytosine. Cytosine methylation occurs throughout the human genome at many CpG sites. Cytosine methylation at CG sites also occurs throughout the genomes of other eukaryotes. In mammals, for example, 70% to 80% of CpG cytosines may be methylated. In pathogenic microorganisms of interest, such as bacteria and viruses, this CpG methylation does not occur or is significantly lower than the CpG methylation in the human genome. Thus, dehosting can be achieved by selectively cleaving CpG methylated DNA.

In some embodiments, the disclosure provides for a dehosting method which comprises a nucleic acid binding protein or binding domain which binds to CpG islands or CpG sites. In another embodiment, the binding domain comprises a protein or fragment thereof that binds to methylated CpG islands. In yet another embodiment, the nucleic acid binding protein binding domain comprises a methyl-CpG-binding domain (MBD). An example of an MBD is a polypeptide of about 70 residues that folds into an alpha/beta sandwich structure comprising a layer of twisted beta sheet, backed by another layer formed by the alpha1 helix and a hairpin loop at the C terminus. These layers are both amphipathic, with the alpha1 helix and the beta sheet lying parallel and the hydrophobic faces tightly packed against each other. The beta sheet is composed of two long inner strands (beta2 and beta3) sandwiched by two shorter outer strands (beta1 and beta4). In a further embodiment, the nucleic acid binding protein or binding domain comprises a protein selected from the group consisting of MECP2, MBD1, MBD2, and MBD4, or a fragment thereof. In yet a further embodiment, the nucleic acid binding protein or binding domain comprises MBD2. In a certain embodiment, the nucleic acid binding protein or binding domain comprises a fragment of MBD2. In another embodiment, the nucleic acid binding protein or binding domain comprises MBD5, MBD6, SETDB1, SETDB2, TIP5/BAZ2A, or BAZ2B, or a fragment thereof. In yet another embodiment, the nucleic acid binding protein or binding domain comprises a CpG methylation or demethylation protein, or a fragment thereof. In a further embodiment, CpG bound nonmicrobial nucleic acids can then be removed from the sample by use of a substrate which comprises an affinity agent that selectively binds to a nucleic acid binding protein or binding domain which binds to CpG islands or CpG sites. Examples of affinity agents include antibodies or antibody fragments that selectively bind to a nucleic acid binding protein or binding domain which binds to CpG islands or CpG sites. Affinity agents comprising antibodies or antibody fragments can be bound to a substrate or alternatively may itself be bound by a second antibody which is bound to a substrate, thereby providing a means to separate and remove the nonmicrobial nucleic acids from a sample.

In another embodiment the disclosure provides for dehosting method that uses a nuclease, or a recombinant protein which comprises a nuclease domain, whereby the nuclease cleaves non-pathogen-based nucleic acids into fragments. In the latter case, the recombinant protein may also comprise a nucleic acid protein binding domain having activity for nucleic acid binding proteins (e.g., histones, methyl-CpG-binding proteins). The nuclease or nuclease can include, but are not limited to, a non-specific nuclease, an endonuclease, non-specific endonuclease, non-specific exonuclease, a homing endonuclease, and restriction endonuclease. In another embodiment, the nuclease domain is derived from any nuclease where the nuclease or nuclease domain does not itself have its own unique target. In yet another embodiment, the nuclease domain has activity when fused to other proteins. Examples of non-specific nucleases include FokI and I-TevI. In some embodiments, the nuclease domain is FokI or a fragment thereof. In a further embodiment, the nuclease domain is I-TevI or a fragment thereof. In yet a further embodiment, the FokI or I-TevI or fragment thereof is unmutated and/or wild-type. Further examples of nucleases include but are not limited to, Deoxyribonuclease I (DNase I), RecBCD endonuclease, T7 endonuclease, T4 endonuclease IV, Bal 31 endonuclease, endonucleaseI (endo I), Micrococcal nuclease, Endonuclease II (endo VI, exo III), Neurospora endonuclease, S1-nuclease, P1-nuclease, Mung bean nuclease I, Ustilago nuclease (Dnase I), AP endonuclease, and Endo R.

The disclosure provides methods for characterizing a target DNA that is present in a sample that has been processed with the universal primers described herein. The methods involve contacting the sample with a type V CRISPR/Cas effector protein and one or more guide RNAs in the presence of detector DNA. The contacting step can generate a protospacer-adjacent motif (PAM)-distal cleavage product comprising a 5′ overhang; and ligating a double-stranded nucleic acid adapter to the cleavage product, to generate a ligation product. The double-stranded nucleic acid adapter comprises a 5′ overhang that comprises a stretch of from 3 to 15 contiguous nucleotides that are complementary to a contiguous stretch of nucleotides of the same length in the 5′ overhang of the PAM-distal cleavage product. The ligation product includes the target DNA, which can be sequenced. The sample can be subjected to one or more amplification steps prior to the contacting step, with universal primers of the disclosure that provide for amplification of nucleic acids of, e.g., microorganisms of a particular taxon. The sample can also be subjected to one or more nucleic acid modification steps before contacting the sample with a type V CRISPR/Cas effector protein and/or prior to ligation. The sample can also be contacted with a ssDNA reporter molecule (a labelled single-stranded detector DNA), a guide RNA, and a type V CRISPR/Cas effector protein, such that, upon contact with a target DNA present in the sample, a signal is produced. For example, in some cases, a method of the disclosure comprises: a) contacting a sample comprising (or suspected of comprising) a target nucleic acid with: i) a type V CRISPR/Cas effector polypeptide; ii) a guide RNA; and iii) a labelled single-stranded detector DNA, where the labelled single-stranded detector DNA produces a signal when the target nucleic acid is present in the sample, and where the contacting step generates a PAM-distal cleavage product comprising a 5′ overhang; and optionally b) ligating a double-stranded nucleic acid adapter to the cleavage product, to generate a ligation product. In some cases, a method of the disclosure comprises: a) contacting a sample comprising (or suspected of comprising) a target nucleic acid with one or more amplification universal primers or universal primer pairs, thereby generating an amplification product(s); b) contacting the sample comprising the amplification product(s) with: i) a type V CRISPR/Cas effector polypeptide; ii) a guide RNA; and iii) a labelled single-stranded detector DNA, where the labelled single-stranded detector DNA produces a signal when the target nucleic acid is present in the sample, and where the contacting step generates a PAM-distal cleavage product comprising a 5′ overhang; and optionally c) ligating a double-stranded nucleic acid adapter to the cleavage product, to generate a ligation product. In some cases, a method of the disclosure comprises: a) contacting a sample comprising (or suspected of comprising) a target ribonucleic acid with a reverse transcriptase, thereby generating a target DNA; b) contacting the sample comprising the target DNA with: i) a type V CRISPR/Cas effector polypeptide; ii) a guide RNA; and iii) a labelled single-stranded detector DNA, where the labelled single-stranded detector DNA produces a signal when the target nucleic acid is present in the sample, and where the contacting step generates a PAM-distal cleavage product comprising a 5′ overhang; and optionally c) ligating a double-stranded nucleic acid adapter to the cleavage product, to generate a ligation product. In some cases, a method of the disclosure comprises: a) contacting a sample comprising (or suspected of comprising) a target ribonucleic acid with a reverse transcriptase, thereby generating a target DNA; b) contacting the sample comprising the target DNA with one or more amplification universal primers or universal primer pairs, thereby generating an amplification product(s); c) contacting the sample comprising the amplification product(s) with: i) a type V CRISPR/Cas effector polypeptide; ii) a guide RNA; and iii) a labelled single-stranded detector DNA, where the labelled single-stranded detector DNA produces a signal when the target nucleic acid is present in the sample, and where the contacting step generates a PAM-distal cleavage product comprising a 5′ overhang; and optionally d) ligating a double-stranded nucleic acid adapter to the cleavage product, to generate a ligation product.

In some cases, a subject method includes contacting a sample (e.g., a sample comprising a target DNA and a plurality of non-target ssDNAs) with: i) a Type V CRISPR/Cas effector protein (e.g., a Cas12 protein such as Cas12a, Cas12b, Cas12c, Cas12d, Cas12e); ii) a guide RNA (or precursor guide RNA array); and iii) a detector DNA that is single stranded and does not hybridize with the guide sequence of the guide RNA. For example, in some cases, a subject method includes contacting a sample with a labeled single stranded detector DNA (detector ssDNA) that includes a fluorescence-emitting dye pair; the Type V CRISPR/Cas effector protein (e.g., a Cas12 protein such as Cas12a, Cas12b, Cas12c, Cas12d, Cas12e) cleaves the labeled detector ssDNA after it is activated (by binding to the guide RNA in the context of the guide RNA hybridizing to a target DNA); and the detectable signal that is measured is produced by the fluorescence-emitting dye pair. For example, in some cases, a subject method includes contacting a sample with a labeled detector ssDNA comprising a fluorescence resonance energy transfer (FRET) pair or a quencher/fluor pair, or both. In some cases, a subject method includes contacting a sample with a labeled detector ssDNA comprising a FRET pair. In some cases, a subject method includes contacting a sample with a labeled detector ssDNA comprising a fluor/quencher pair.

Fluorescence-emitting dye pairs comprise a FRET pair or a quencher/fluor pair. In both cases of a FRET pair and a quencher/fluor pair, the emission spectrum of one of the dyes overlaps a region of the absorption spectrum of the other dye in the pair. As used herein, the term “fluorescence-emitting dye pair” is a generic term used to encompass both a “fluorescence resonance energy transfer (FRET) pair” and a “quencher/fluor pair,” both of which terms are discussed in more detail below. The term “fluorescence-emitting dye pair” is used interchangeably with the phrase “a FRET pair and/or a quencher/fluor pair.”

In some cases (e.g., when the detector ssDNA includes a FRET pair) the labeled detector ssDNA produces an amount of detectable signal prior to being cleaved, and the amount of detectable signal that is measured is reduced when the labeled detector ssDNA is cleaved. In some cases, the labeled detector ssDNA produces a first detectable signal prior to being cleaved (e.g., from a FRET pair) and a second detectable signal when the labeled detector ssDNA is cleaved (e.g., from a quencher/fluor pair). As such, in some cases, the labeled detector ssDNA comprises a FRET pair and a quencher/fluor pair.

In some cases, the labeled detector ssDNA comprises a FRET pair. FRET is a process by which radiationless transfer of energy occurs from an excited state fluorophore to a second chromophore in close proximity. The range over which the energy transfer can take place is limited to approximately 10 nanometers (100 angstroms), and the efficiency of transfer is extremely sensitive to the separation distance between fluorophores. Thus, as used herein, the term “FRET” (“fluorescence resonance energy transfer”; also known as “Forster resonance energy transfer”) refers to a physical phenomenon involving a donor fluorophore and a matching acceptor fluorophore selected so that the emission spectrum of the donor overlaps the excitation spectrum of the acceptor, and further selected so that when donor and acceptor are in close proximity (usually 10 nm or less) to one another, excitation of the donor will cause excitation of and emission from the acceptor, as some of the energy passes from donor to acceptor via a quantum coupling effect. Thus, a FRET signal serves as a proximity gauge of the donor and acceptor; only when they are in close proximity to one another is a signal generated. The FRET donor moiety (e.g., donor fluorophore) and FRET acceptor moiety (e.g., acceptor fluorophore) are collectively referred to herein as a “FRET pair”.

The donor-acceptor pair (a FRET donor moiety and a FRET acceptor moiety) is referred to herein as a “FRET pair” or a “signal FRET pair.” Thus, in some cases, a subject labeled detector ssDNA includes two signal partners (a signal pair), when one signal partner is a FRET donor moiety and the other signal partner is a FRET acceptor moiety. A subject labeled detector ssDNA that includes such a FRET pair (a FRET donor moiety and a FRET acceptor moiety) will thus exhibit a detectable signal (a FRET signal) when the signal partners are in close proximity (e.g., while on the same RNA molecule), but the signal will be reduced (or absent) when the partners are separated (e.g., after cleavage of the RNA molecule by a Type V CRISPR/Cas effector protein (e.g., a Cas12 protein such as Cas12a, Cas12b, Cas12c, Cas12d, Cas12e)).

FRET donor and acceptor moieties (FRET pairs) will be known to one of ordinary skill in the art and any convenient FRET pair (e.g., any convenient donor and acceptor moiety pair) can be used. See also: Bajar et al. Sensors (Basel). 2016 Sep. 14; 16(9); and Abraham et al. PLoS One. 2015 Aug. 3; 10(8):e0134436.

In some cases, a detectable signal is produced when the labeled detector ssDNA is cleaved (e.g., in some cases, the labeled detector ssDNA comprises a quencher/fluor pair). One signal partner of a signal quenching pair produces a detectable signal and the other signal partner is a quencher moiety that quenches the detectable signal of the first signal partner (i.e., the quencher moiety quenches the signal of the signal moiety such that the signal from the signal moiety is reduced (quenched) when the signal partners are in proximity to one another, e.g., when the signal partners of the signal pair are in close proximity).

For example, in some cases, an amount of detectable signal increases when the labeled detector ssDNA is cleaved. For example, in some cases, the signal exhibited by one signal partner (a signal moiety) is quenched by the other signal partner (a quencher signal moiety), e.g., when both are present on the same ssDNA molecule prior to cleavage by a Type V CRISPR/Cas effector protein (e.g., a Cas12 protein such as Cas12a, Cas12b, Cas12c, Cas12d, Cas12e). Such a signal pair is referred to herein as a “quencher/fluor pair”, “quenching pair”, or “signal quenching pair.” For example, in some cases, one signal partner (e.g., the first signal partner) is a signal moiety that produces a detectable signal that is quenched by the second signal partner (e.g., a quencher moiety). The signal partners of such a quencher/fluor pair will thus produce a detectable signal when the partners are separated (e.g., after cleavage of the detector ssDNA by a Type V CRISPR/Cas effector protein (e.g., a Cas12 protein such as Cas12a, Cas12b, Cas12c, Cas12d, Cas12e)), but the signal will be quenched when the partners are in close proximity (e.g., prior to cleavage of the detector ssDNA by a Type V CRISPR/Cas effector protein (e.g., a Cas12 protein such as Cas12a, Cas12b, Cas12c, Cas12d, Cas12e)).

A quencher moiety can quench a signal from the signal moiety (e.g., prior to cleave of the detector ssDNA by a Type V CRISPR/Cas effector protein (e.g., a Cas12 protein such as Cas12a, Cas12b, Cas12c, Cas12d, Cas12e)) to various degrees. In some cases, a quencher moiety quenches the signal from the signal moiety where the signal detected in the presence of the quencher moiety (when the signal partners are in proximity to one another) is 95% or less of the signal detected in the absence of the quencher moiety (when the signal partners are separated). For example, in some cases, the signal detected in the presence of the quencher moiety can be 90% or less, 80% or less, 70% or less, 60% or less, 50% or less, 40% or less, 30% or less, 20% or less, 15% or less, 10% or less, or 5% or less of the signal detected in the absence of the quencher moiety. In some cases, no signal (e.g., above background) is detected in the presence of the quencher moiety.

In some cases, the signal detected in the absence of the quencher moiety (when the signal partners are separated) is at least 1.2 fold greater (e.g., at least 1.3 fold, at least 1.5 fold, at least 1.7 fold, at least 2 fold, at least 2.5 fold, at least 3 fold, at least 3.5 fold, at least 4 fold, at least 5 fold, at least 7 fold, at least 10 fold, at least 20 fold, or at least 50 fold greater) than the signal detected in the presence of the quencher moiety (when the signal partners are in proximity to one another).

In some cases, the signal moiety is a fluorescent label. In some such cases, the quencher moiety quenches the signal (the light signal) from the fluorescent label (e.g., by absorbing energy in the emission spectra of the label). Thus, when the quencher moiety is not in proximity with the signal moiety, the emission (the signal) from the fluorescent label is detectable because the signal is not absorbed by the quencher moiety. Any convenient donor acceptor pair (signal moiety/quencher moiety pair) can be used and many suitable pairs are known in the art.

In some cases the quencher moiety absorbs energy from the signal moiety (also referred to herein as a “detectable label”) and then emits a signal (e.g., light at a different wavelength). Thus, in some cases, the quencher moiety is itself a signal moiety (e.g., a signal moiety can be 6-carboxyfluorescein while the quencher moiety can be 6-carboxy-tetramethylrhodamine), and in some such cases, the pair could also be a FRET pair. In some cases, a quencher moiety is a dark quencher. A dark quencher can absorb excitation energy and dissipate the energy in a different way (e.g., as heat). Thus, a dark quencher has minimal to no fluorescence of its own (does not emit fluorescence). Examples of dark quenchers are further described in U.S. Pat. Nos. 8,822,673 and 8,586,718; U.S. patent publications 20140378330, 20140349295, and 20140194611; and international patent applications: WO200142505 and WO200186001, all if which are hereby incorporated by reference in their entirety.

Examples of fluorescent labels include, but are not limited to: an Alexa Fluor® dye, an ATTO dye (e.g., ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATTO 740), a DyLight dye, a cyanine dye (e.g., Cy2, Cy3, Cy3.5, Cy3b, Cy5, Cy5.5, Cy7, Cy7.5), a FluoProbes dye, a Sulfo Cy dye, a Seta dye, an IRIS Dye, a SeTau dye, an SRfluor dye, a Square dye, fluorescein isothiocyanate (FITC), tetramethylrhodamine (TRITC), Texas Red, Oregon Green, Pacific Blue, Pacific Green, Pacific Orange, quantum dots, and a tethered fluorescent protein.

In some cases, a detectable label is a fluorescent label selected from: an Alexa Fluor® dye, an ATTO dye (e.g., ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATTO 740), a DyLight dye, a cyanine dye (e.g., Cy2, Cy3, Cy3.5, Cy3b, Cy5, Cy5.5, Cy7, Cy7.5), a FluoProbes dye, a Sulfo Cy dye, a Seta dye, an IRIS Dye, a SeTau dye, an SRfluor dye, a Square dye, fluorescein (FITC), tetramethylrhodamine (TRITC), Texas Red, Oregon Green, Pacific Blue, Pacific Green, and Pacific Orange.

In some cases, a detectable label is a fluorescent label selected from: an Alexa Fluor® dye, an ATTO dye (e.g., ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATTO 740), a DyLight dye, a cyanine dye (e.g., Cy2, Cy3, Cy3.5, Cy3b, Cy5, Cy5.5, Cy7, Cy7.5), a FluoProbes dye, a Sulfo Cy dye, a Seta dye, an IRIS Dye, a SeTau dye, an SRfluor dye, a Square dye, fluorescein (FITC), tetramethylrhodamine (TRITC), Texas Red, Oregon Green, Pacific Blue, Pacific Green, Pacific Orange, a quantum dot, and a tethered fluorescent protein.

Examples of ATTO dyes include, but are not limited to: ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, and ATTO 740.

Examples of AlexaFluor dyes include, but are not limited to: Alexa Fluor® 350, Alexa Fluor® 405, Alexa Fluor® 430, Alexa Fluor® 488, Alexa Fluor® 500, Alexa Fluor® 514, Alexa Fluor® 532, Alexa Fluor® 546, Alexa Fluor® 555, Alexa Fluor® 568, Alexa Fluor® 594, Alexa Fluor® 610, Alexa Fluor® 633, Alexa Fluor® 635, Alexa Fluor® 647, Alexa Fluor® 660, Alexa Fluor® 680, Alexa Fluor® 700, Alexa Fluor® 750, Alexa Fluor® 790, and the like.

Examples of quencher moieties include, but are not limited to: a dark quencher, a Black Hole Quencher® (BHQ®.) (e.g., BHQ-0, BHQ-1, BHQ-2, BHQ-3), a Qx1 quencher, an ATTO quencher (e.g., ATTO 540Q, ATTO 580Q, and ATTO 612Q), dimethylaminoazobenzenesulfonic acid (Dabsyl), Iowa Black RQ, Iowa Black FQ, IRDye QC-1, a QSY dye (e.g., QSY 7, QSY 9, QSY 21), AbsoluteQuencher, Eclipse, and metal clusters such as gold nanoparticles, and the like.

In some cases, a quencher moiety is selected from: a dark quencher, a Black Hole Quencher® (BHQ®) (e.g., BHQ-0, BHQ-1, BHQ-2, BHQ-3), a Qx1 quencher, an ATTO quencher (e.g., ATTO 540Q, ATTO 580Q, and ATTO 612Q), dimethylaminoazobenzenesulfonic acid (Dabsyl), Iowa Black RQ, Iowa Black FQ, IRDye QC-1, a QSY dye (e.g., QSY 7, QSY 9, QSY 21), AbsoluteQuencher, Eclipse, and a metal cluster.

Examples of an ATTO quencher include, but are not limited to: ATTO 540Q, ATTO 580Q, and ATTO 612Q. Examples of a Black Hole Quencher® (BHQ®) include, but are not limited to: BHQ-0 (493 nm), BHQ-1 (534 nm), BHQ-2 (579 nm) and BHQ-3 (672 nm).

In some cases, cleavage of a labeled detector ssDNA can be detected by measuring a colorimetric read-out. For example, the liberation of a fluorophore (e.g., liberation from a FRET pair, liberation from a quencher/fluor pair, and the like) can result in a wavelength shift (and thus color shift) of a detectable signal. Thus, in some cases, cleavage of a subject labeled detector ssDNA can be detected by a color-shift. Such a shift can be expressed as a loss of an amount of signal of one color (wavelength), a gain in the amount of another color, a change in the ration of one color to another, and the like.

As noted above, in some cases, the ligation product is sequenced; e.g., the target DNA present in the ligation product is sequenced. The contacting, ligating, and sequencing steps can be carried out in a single reaction container (also referred to herein as a “reaction vessel”). Thus, in a single reaction container, a target DNA can be both detected and sequenced. In some cases, the contacting and ligating steps are carried out in a first reaction container; and the sequencing step is carried out in a second reaction container.

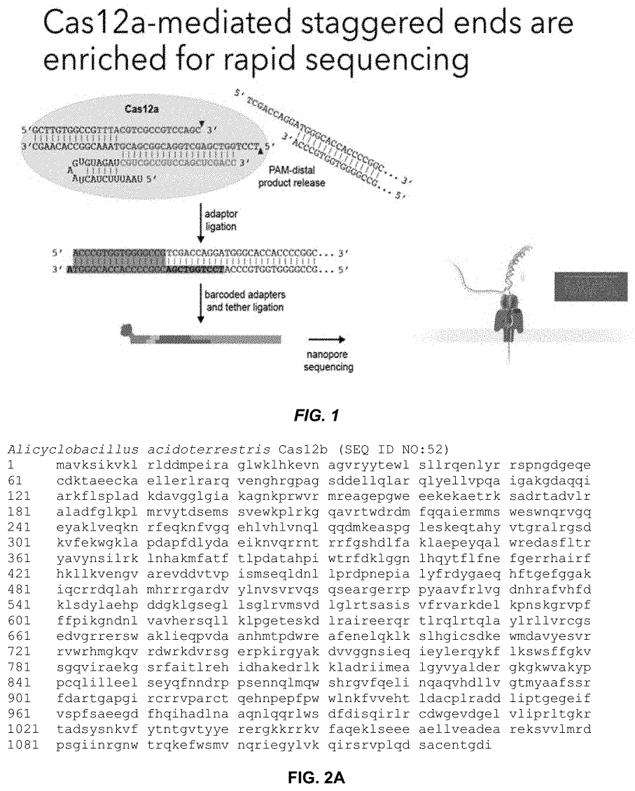

Cleavage of a target DNA with a type V CRISPR/Cas effector polypeptide (e.g., a Cas12 polypeptide) generates a cleavage product having a 5′ overhang of from about 4 nucleotides to about 12 nucleotides in length. This 5′ overhang can provide a point of hybridization for an adapter molecule (e.g., a double-stranded nucleic acid adapter) having a 5′ overhang with a nucleotide sequence that is at least partially complementary to the nucleotide sequence of the 5′ overhang of the type V CRISPR/Cas effector polypeptide cleavage product. The adapter molecule can be ligated to the type V CRISPR/Cas effector polypeptide cleavage product, generating a type V CRISPR/Cas effector polypeptide cleavage product/adapter hybrid nucleic acid. The type V CRISPR/Cas effector polypeptide cleavage product/adapter hybrid nucleic acid can be ligated to one or more additional adapters, e.g., an adapter that provides a bar code, an adapter that allows for next-generation sequencing, and the like. For example, the one or more additional adapters can include a nucleotide sequence specific for coupling to a sequencing platform; such an adapter may also include a barcode sequence. In some cases, the additional adapter comprises a nucleotide sequence that is at least 70% identical to a support-bound oligonucleotide conjugated to a solid support; in some cases, the solid support is coupled to a sequencing platform. In some cases, the additional adapter comprises a binding site for a sequencing primer. Ligation of various adapters to a type V CRISPR/Cas effector polypeptide cleavage product is depicted schematically in FIG. 1.

The contacting step of a subject method can be carried out in a composition comprising divalent metal ions. The contacting step can be carried out in an acellular environment, e.g., outside of a cell. The contacting step can be carried out inside a cell. The contacting step can be carried out in a cell in vitro. The contacting step can be carried out in a cell ex vivo. The contacting step can be carried out in a cell in vivo.

As noted above, nucleic acid(s) present in a sample can be subjected to one or more nucleic acid modification steps before contacting the sample with a type V CRISPR/Cas effector protein and/or prior to ligation. For example, in some cases, a dsDNA can be subjected to dephosphorylation prior to cleavage with a type V CRISPR/Cas effector protein. The dephosphorylation step would avoid sequencing of dsDNA not cleaved by the type V CRISPR/Cas effector protein. As another example, in some cases, a dsDNA is cleaved with a type V CRISPR/Cas effector protein and, prior to ligation with a double-stranded nucleic acid adapter, the cleavage product is subjected to Klenow repair of overhangs, e.g., to fill in a 3′ overhang.

The guide RNA can be provided as RNA or as a nucleic acid encoding the guide RNA (e.g., a DNA such as a recombinant expression vector). The Type V CRISPR/Cas effector protein (e.g., a Cas12 protein such as Cas12a, Cas12b, Cas12c, Cas12d, Cas12e) can be provided as a protein or as a nucleic acid encoding the protein (e.g., an mRNA, a DNA such as a recombinant expression vector). In some cases, two or more (e.g., 3 or more, 4 or more, 5 or more, or 6 or more) guide RNAs can be provided by (e.g., using a precursor guide RNA array, which can be cleaved by the Type V CRISPR/Cas effector protein into individual (“mature”) guide RNAs).

In some cases (e.g., when contacting with a guide RNA and a Type V CRISPR/Cas effector protein (e.g., a Cas12 protein such as Cas12a, Cas12b, Cas12c, Cas12d, Cas12e)), the sample is contacted for 2 hours or less (e.g., 1.5 hours or less, 1 hour or less, 40 minutes or less, 30 minutes or less, 20 minutes or less, 10 minutes or less, or 5 minutes or less, or 1 minute or less) prior to the ligating step. For example, in some cases, the sample is contacted for 40 minutes or less prior to the ligating step. In some cases, the sample is contacted for 20 minutes or less prior to the ligating step. In some cases, the sample is contacted for 10 minutes or less prior to the ligating step. In some cases, the sample is contacted for 5 minutes or less prior to the ligating step. In some cases, the sample is contacted for 1 minute or less prior to the ligating step. In some cases, the sample is contacted for from 50 seconds to 60 seconds prior to the ligating step. In some cases, the sample is contacted for from 40 seconds to 50 seconds prior to the ligating step. In some cases, the sample is contacted for from 30 seconds to 40 seconds prior to the ligating step. In some cases, the sample is contacted for from 20 seconds to 30 seconds prior to the ligating step. In some cases, the sample is contacted for from 10 seconds to 20 seconds prior to the ligating step.

A method of the disclosure for characterizing a target DNA (single-stranded or double-stranded) in a sample can provide for characterization of a target DNA with a high degree of sensitivity. In some cases, a method of the disclosure can be used to characterize a target DNA present in a sample comprising a plurality of DNAs (including the target DNA and a plurality of non-target DNAs), where the target DNA is present at one or more copies per 10⁷ non-target DNAs (e.g., one or more copies per 10⁶ non-target DNAs, one or more copies per 10⁵ non-target DNAs, one or more copies per 10⁴ non-target DNAs, one or more copies per 10³ non-target DNAs, one or more copies per 10² non-target DNAs, one or more copies per 50 non-target DNAs, one or more copies per 20 non-target DNAs, one or more copies per 10 non-target DNAs, or one or more copies per 5 non-target DNAs). In some cases, a method of the disclosure can be used to characterize a target DNA present in a sample comprising a plurality of DNAs (including the target DNA and a plurality of non-target DNAs), where the target DNA is present at one or more copies per 10¹⁸ non-target DNAs (e.g., one or more copies per 10¹⁵ non-target DNAs, one or more copies per 10¹² non-target DNAs, one or more copies per 10⁹ non-target DNAs, one or more copies per 10⁶ non-target DNAs, one or more copies per 10⁵ non-target DNAs, one or more copies per 10⁴ non-target DNAs, one or more copies per 10³ non-target DNAs, one or more copies per 10² non-target DNAs, one or more copies per 50 non-target DNAs, one or more copies per 20 non-target DNAs, one or more copies per 10 non-target DNAs, or one or more copies per 5 non-target DNAs).

In some cases, a method of the disclosure can characterize a target DNA present in a sample, where the target DNA is present at from one copy per 10⁷ non-target DNAs to one copy per 10 non-target DNAs (e.g., from 1 copy per 10⁷ non-target DNAs to 1 copy per 10² non-target DNAs, from 1 copy per 10⁷ non-target DNAs to 1 copy per 10³ non-target DNAs, from 1 copy per 10⁷ non-target DNAs to 1 copy per 10⁴ non-target DNAs, from 1 copy per 10⁷ non-target DNAs to 1 copy per 10⁵ non-target DNAs, from 1 copy per 10⁷ non-target DNAs to 1 copy per 10⁶ non-target DNAs, from 1 copy per 10⁶ non-target DNAs to 1 copy per 10 non-target DNAs, from 1 copy per 10⁶ non-target DNAs to 1 copy per 10² non-target DNAs, from 1 copy per 10⁶ non-target DNAs to 1 copy per 10³ non-target DNAs, from 1 copy per 10⁶ non-target DNAs to 1 copy per 10⁴ non-target DNAs, from 1 copy per 10⁶ non-target DNAs to 1 copy per 10⁵ non-target DNAs, from 1 copy per 10⁵ non-target DNAs to 1 copy per 10 non-target DNAs, from 1 copy per 10⁵ non-target DNAs to 1 copy per 10² non-target DNAs, from 1 copy per 10⁵ non-target DNAs to 1 copy per 10³ non-target DNAs, or from 1 copy per 10⁵ non-target DNAs to 1 copy per 10⁴ non-target DNAs).

In some cases, a method of the disclosure can characterize a target DNA present in a sample, where the target DNA is present at from one copy per 10¹⁸ non-target DNAs to one copy per 10 non-target DNAs (e.g., from 1 copy per 10¹⁸ non-target DNAs to 1 copy per 10² non-target DNAs, from 1 copy per 10¹⁵ non-target DNAs to 1 copy per 10² non-target DNAs, from 1 copy per 10¹² non-target DNAs to 1 copy per 10² non-target DNAs, from 1 copy per 10⁹ non-target DNAs to 1 copy per 10² non-target DNAs, from 1 copy per 10⁷ non-target DNAs to 1 copy per 10² non-target DNAs, from 1 copy per 10⁷ non-target DNAs to 1 copy per 10³ non-target DNAs, from 1 copy per 10⁷ non-target DNAs to 1 copy per 10⁴ non-target DNAs, from 1 copy per 10⁷ non-target DNAs to 1 copy per 10⁵ non-target DNAs, from 1 copy per 10⁷ non-target DNAs to 1 copy per 10⁶ non-target DNAs, from 1 copy per 10⁶ non-target DNAs to 1 copy per 10 non-target DNAs, from 1 copy per 10⁶ non-target DNAs to 1 copy per 10² non-target DNAs, from 1 copy per 10⁶ non-target DNAs to 1 copy per 10³ non-target DNAs, from 1 copy per 10⁶ non-target DNAs to 1 copy per 10⁴ non-target DNAs, from 1 copy per 10⁶ non-target DNAs to 1 copy per 10⁵ non-target DNAs, from 1 copy per 10⁵ non-target DNAs to 1 copy per 10 non-target DNAs, from 1 copy per 10⁵ non-target DNAs to 1 copy per 10² non-target DNAs, from 1 copy per 10⁵ non-target DNAs to 1 copy per 10³ non-target DNAs, or from 1 copy per 10⁵ non-target DNAs to 1 copy per 10⁴ non-target DNAs).

In some cases, a method of the disclosure can characterize a target DNA present in a sample, where the target DNA is present at from one copy per 10⁷ non-target DNAs to one copy per 100 non-target DNAs (e.g., from 1 copy per 10⁷ non-target DNAs to 1 copy per 10² non-target DNAs, from 1 copy per 10⁷ non-target DNAs to 1 copy per 10³ non-target DNAs, from 1 copy per 10⁷ non-target DNAs to 1 copy per 10⁴ non-target DNAs, from 1 copy per 10⁷ non-target DNAs to 1 copy per 10⁵ non-target DNAs, from 1 copy per 10⁷ non-target DNAs to 1 copy per 10⁶ non-target DNAs, from 1 copy per 10⁶ non-target DNAs to 1 copy per 100 non-target DNAs, from 1 copy per 10⁶ non-target DNAs to 1 copy per 10² non-target DNAs, from 1 copy per 10⁶ non-target DNAs to 1 copy per 10³ non-target DNAs, from 1 copy per 10⁶ non-target DNAs to 1 copy per 10⁴ non-target DNAs, from 1 copy per 10⁶ non-target DNAs to 1 copy per 10⁵ non-target DNAs, from 1 copy per 10⁵ non-target DNAs to 1 copy per 100 non-target DNAs, from 1 copy per 10⁵ non-target DNAs to 1 copy per 10² non-target DNAs, from 1 copy per 10⁵ non-target DNAs to 1 copy per 10³ non-target DNAs, or from 1 copy per 10⁵ non-target DNAs to 1 copy per 10⁴ non-target DNAs).

In some cases, the target DNA is present in the sample at a concentration of 10 nM or less, e.g., from about 1 attomolar (aM) to about 100 aM, from about 100 aM to about 500 aM, from about 500 aM to about 1 femtomolar (fM), from about 1 fM to about 100 fM, from about 100 fM to about 500 fM, from about 500 fM to about 1 picomolar (pM), from about 1 pM to about 100 pM, from about 100 pM to about 500 pM, from about 500 pM to about 1 nanomolar (nM), from about 1 nM to about 100 nM, from about 100 nM to about 500 nM, or from about 500 nM to about 1 μM, or more than 1 μM.

In some cases, the target DNA is present in the sample at a concentration of from 500 fM to 1 nM (e.g., from 500 fM to 500 pM, from 500 fM to 200 pM, from 500 fM to 100 pM, from 500 fM to 10 pM, from 500 fM to 1 pM, from 800 fM to 1 nM, from 800 fM to 500 pM, from 800 fM to 200 pM, from 800 fM to 100 pM, from 800 fM to 10 pM, from 800 fM to 1 pM, from 1 pM to 1 nM, from 1 pM to 500 pM, from 1 pM to 200 pM, from 1 pM to 100 pM, or from 1 pM to 10 pM).

As noted above, a method of the disclosure includes use of a Type V CRISPR/Cas effector polypeptide, where a sample that comprises, or is suspected of comprising, a target DNA is contacted with a Type V CRISPR/Cas effector polypeptide and one or more guide RNAs. Type V CRISPR/Cas effector proteins are a subtype of Class 2 CRISPR/Cas effector proteins. For examples of type V CRISPR/Cas systems and their effector proteins (e.g., Cas12 family proteins such as Cas12a), see, e.g., Shmakov et al., Nat Rev Microbiol. 2017 March; 15(3):169-182: “Diversity and evolution of class 2 CRISPR-Cas systems.” Examples include, but are not limited to: Cas12 family (Cas12a, Cas12b, Cas12c), C2c4, C2c8, C2c5, C2c10, and C2c9; as well as CasX (Cas12e) and CasY (Cas12d). Also see, e.g., Koonin et al., Curr Opin Microbiol. 2017 June; 37:67-78: “Diversity, classification and evolution of CRISPR-Cas systems.”

In some cases, a type V CRISPR/Cas effector protein suitable for use in a method of the disclosure is a Cas12 protein (e.g., Cas12a, Cas12b, Cas12c). In some cases, a suitable type V CRISPR/Cas effector protein is a Cas12 protein such as Cas12a, Cas12b, Cas12c, Cas12d, Cas12e, Cas12d, or Cas12e. In some cases, a suitable type V CRISPR/Cas effector protein is a Cas12a protein. In some cases, a suitable type V CRISPR/Cas effector protein is a Cas12b protein. In some cases, a suitable type V CRISPR/Cas effector protein is a Cas12c protein. In some cases, a suitable type V CRISPR/Cas effector protein is a Cas12d protein. In some cases, a suitable type V CRISPR/Cas effector protein is a Cas12e protein. In some cases, a suitable type V CRISPR/Cas effector protein is protein selected from: Cas12 (e.g., Cas12a, Cas12b, Cas12c, Cas12d, Cas12e), C2c4, C2c8, C2c5, C2c10, and C2c9. In some cases, a suitable type V CRISPR/Cas effector protein is protein selected from: C2c4, C2c8, C2c5, C2c10, and C2c9. In some cases, a suitable type V CRISPR/Cas effector protein is protein selected from: C2c4, C2c8, and C2c5. In some cases, a suitable type V CRISPR/Cas effector protein is protein selected from: C2c10 and C2c9.

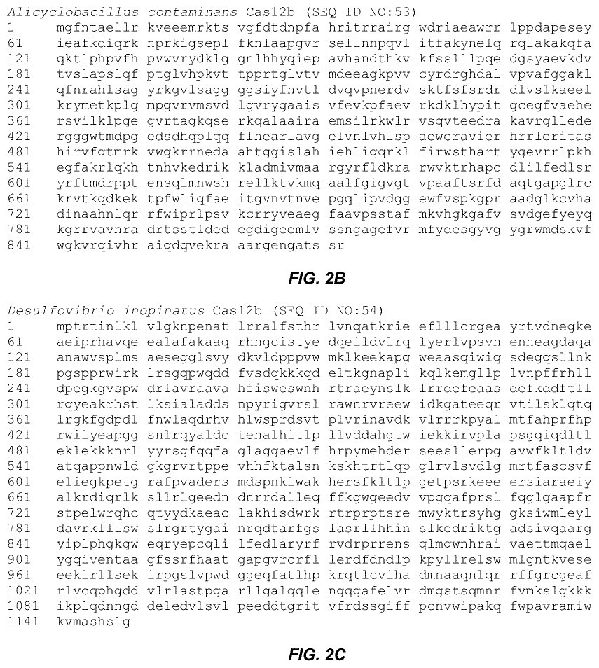

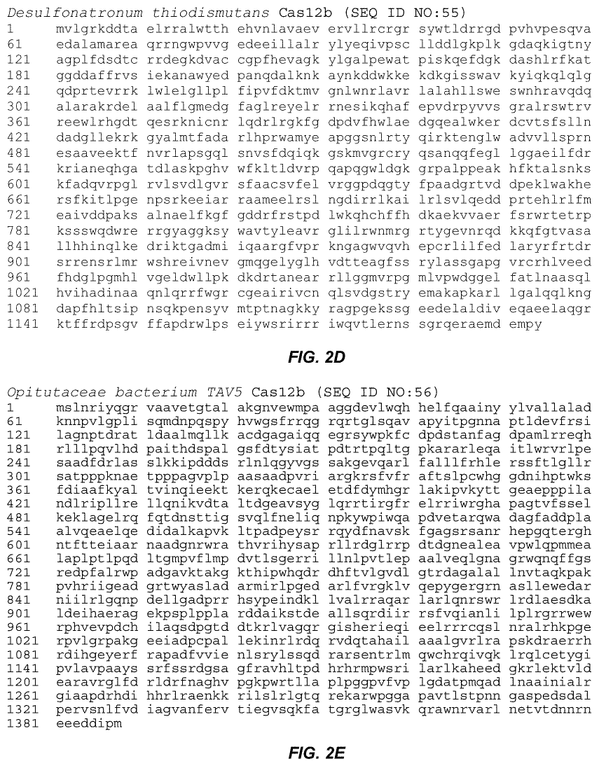

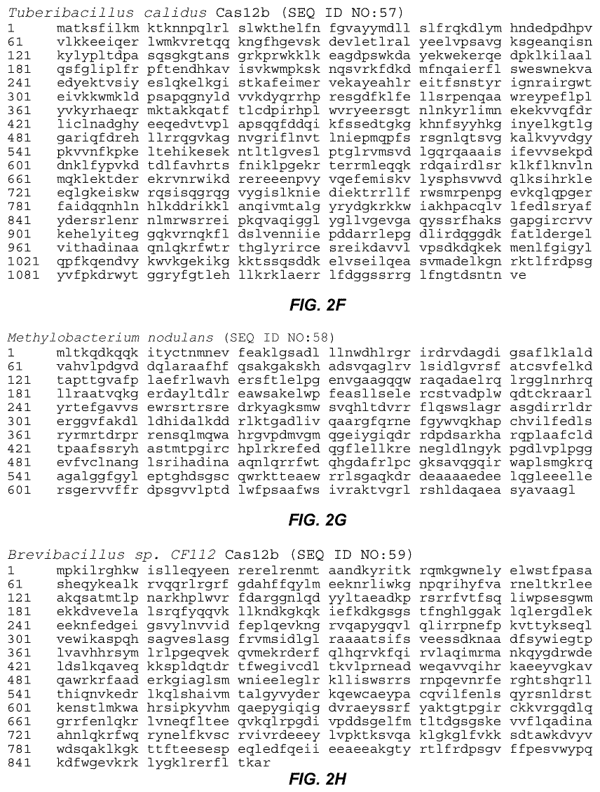

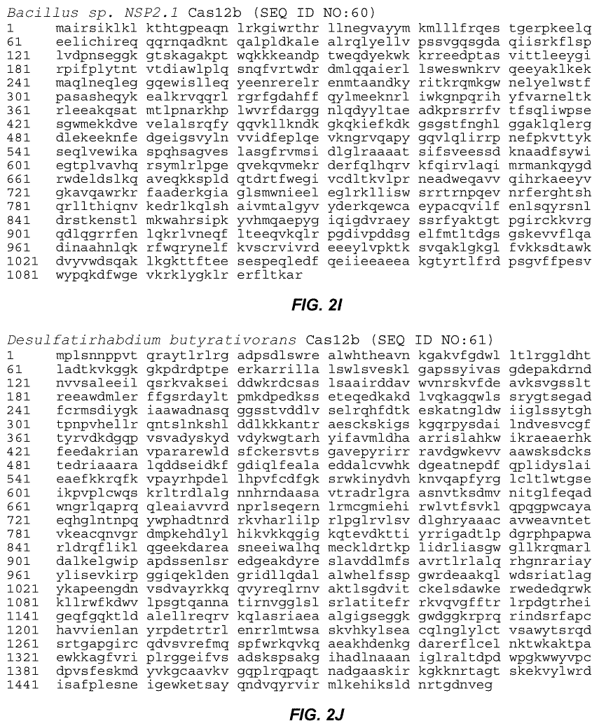

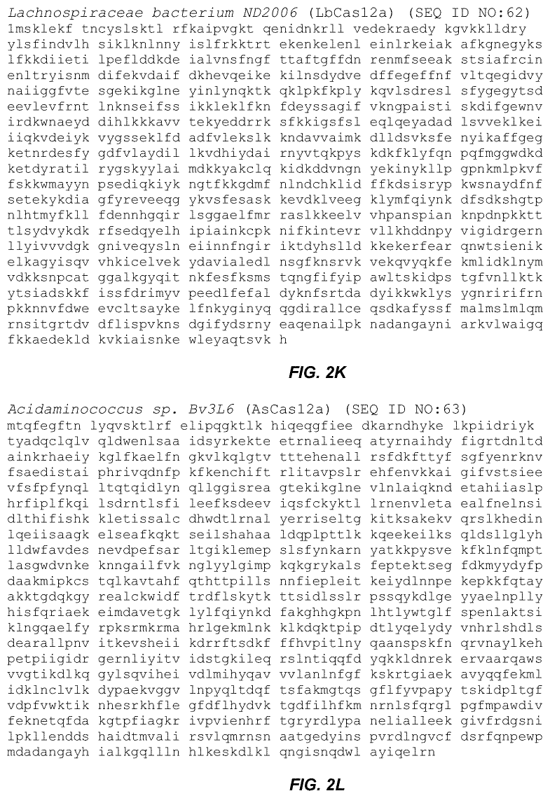

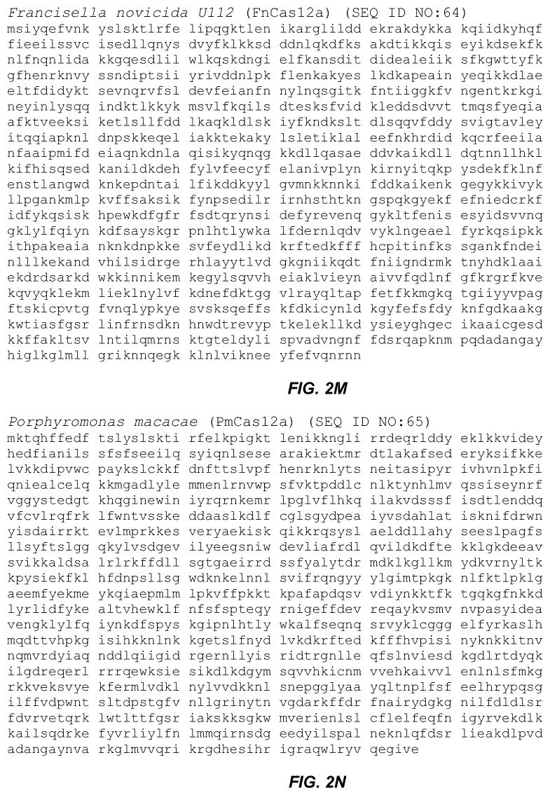

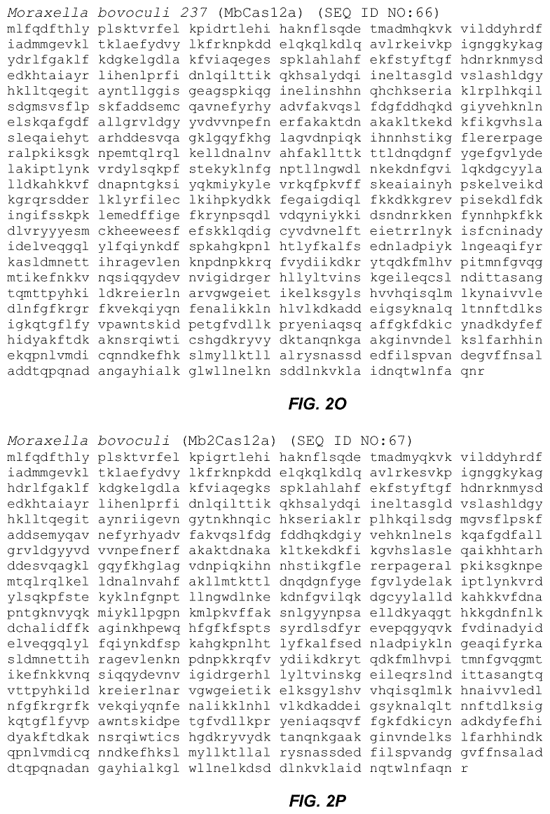

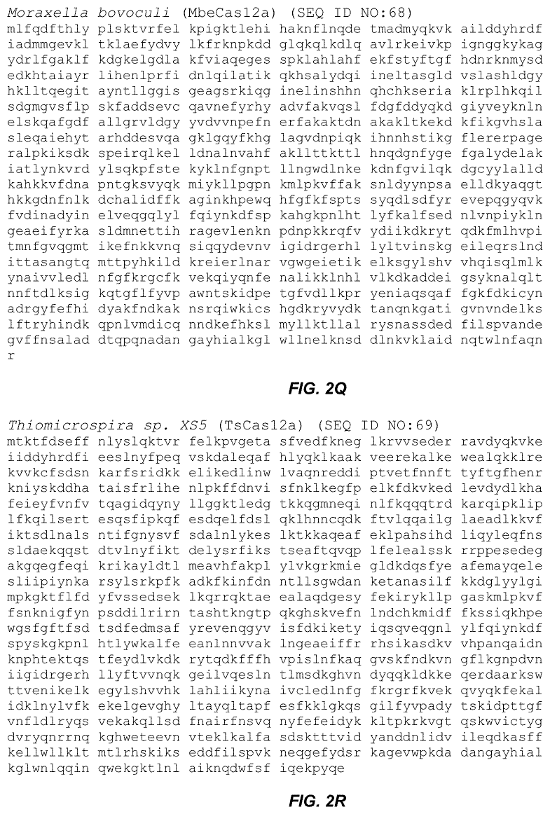

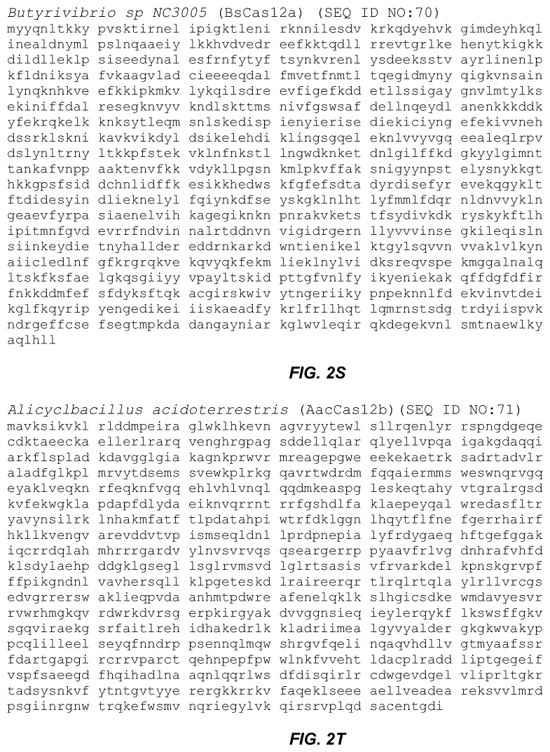

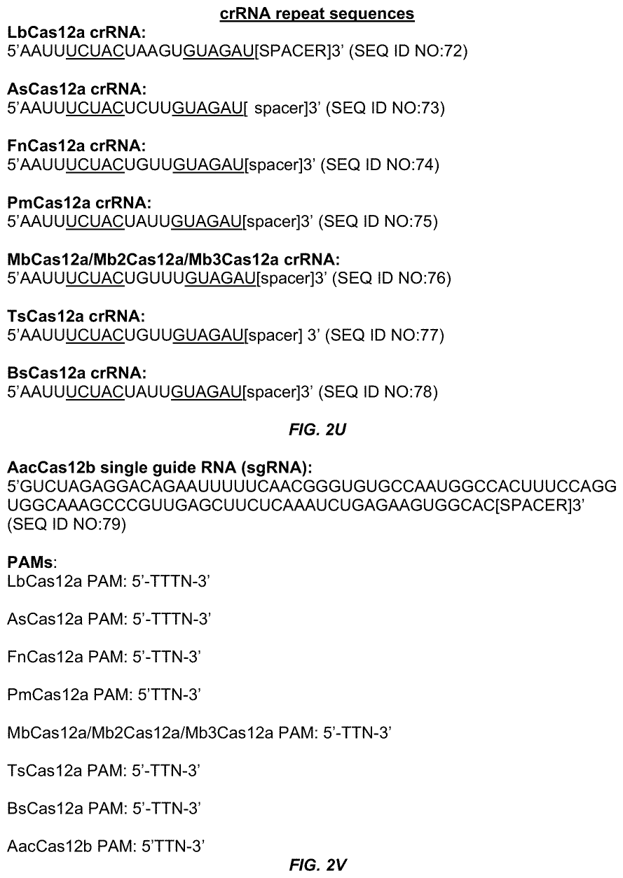

In some cases, a suitable type V CRISPR/Cas effector protein is a naturally-occurring protein (e.g., naturally occurs in prokaryotic cells). In other cases, the Type V CRISPR/Cas effector protein is not a naturally-occurring polypeptide (e.g., the effector protein is a variant protein, a chimeric protein, includes a fusion partner, and the like). Examples of naturally occurring Type V CRISPR/Cas effector proteins include, but are not limited to, those depicted in FIG. 2 (e.g., FIG. 2A-2T). Any Type V CRISPR/Cas effector protein can be suitable for the compositions (e.g., nucleic acids, kits, etc.) and methods of the disclosure (e.g., as long as the Type V CRISPR/Cas effector protein forms a complex with a guide RNA and exhibits ssDNA cleavage activity of non-target ssDNAs once it is activated (by hybridization of and associated guide RNA to its target DNA).

In some cases, a type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with a Cas12 protein (e.g., Cas12a, Cas12b, Cas12c) (e.g., a Cas12 protein depicted in any one of FIG. 2A-2T). For example, in some cases, a type V CRISPR/Cas effector protein comprises an amino acid sequence having 50% or more sequence identity (e.g., 60% or more, 70% or more, 80% or more, 85% or more, 90% or more, 95% or more, 97% or more, 98% or more, 99% or more, or 100% sequence identity) with a Cas12 protein (e.g., Cas12a, Cas12b, Cas12c) (e.g., a Cas12 protein depicted in FIG. 2). In some cases, a type V CRISPR/Cas effector protein comprises an amino acid sequence having 80% or more sequence identity (e.g., 85% or more, 90% or more, 95% or more, 97% or more, 98% or more, 99% or more, or 100% sequence identity) with a Cas12 protein (e.g., Cas12a, Cas12b, Cas12c) (e.g., a Cas12 protein depicted in any one of FIG. 2A-2T). In some cases, a type V CRISPR/Cas effector protein comprises an amino acid sequence having 90% or more sequence identity (e.g., 95% or more, 97% or more, 98% or more, 99% or more, or 100% sequence identity) with a Cas12 protein (e.g., Cas12a, Cas12b, Cas12c) (e.g., a Cas12 protein depicted in FIG. 2). In some cases, a type V CRISPR/Cas effector protein comprises a Cas12 amino acid sequence (e.g., Cas12a, Cas12b, Cas12c) depicted in any one of FIG. 2A-2T.

In some cases, a type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with a Cas12a protein (e.g., a Cas12a protein depicted in any FIG. 2). For example, in some cases, a type V CRISPR/Cas effector protein comprises an amino acid sequence having 50% or more sequence identity (e.g., 60% or more, 70% or more, 80% or more, 85% or more, 90% or more, 95% or more, 97% or more, 98% or more, 99% or more, or 100% sequence identity) with a Cas12a protein (e.g., a Cas12a protein depicted in FIG. 2). In some cases, a type V CRISPR/Cas effector protein comprises an amino acid sequence having 80% or more sequence identity (e.g., 85% or more, 90% or more, 95% or more, 97% or more, 98% or more, 99% or more, or 100% sequence identity) with a Cas12a protein (e.g., a Cas12a protein depicted in FIG. 2). In some cases, a type V CRISPR/Cas effector protein comprises an amino acid sequence having 90% or more sequence identity (e.g., 95% or more, 97% or more, 98% or more, 99% or more, or 100% sequence identity) with a Cas12a protein (e.g., a Cas12a protein depicted in FIG. 2). In some cases, a type V CRISPR/Cas effector protein comprises a Cas12a amino acid sequence depicted in FIG. 2.

In some cases, a suitable type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Lachnospiraceae bacterium ND2006 Cas12a protein amino acid sequence depicted in FIG. 2. In some cases, a suitable type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Acidaminococcus spBV3L6 Cas12a protein amino acid sequence depicted in FIG. 2. In some cases, a suitable type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Francisella novicida U112 Cas12a protein amino acid sequence depicted in FIG. 2. In some cases, a suitable type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Porphyromonas macacae Cas12a protein amino acid sequence depicted in FIG. 2. In some cases, a suitable type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Moraxella bovoculi 237 Cas12a protein amino acid sequence depicted in FIG. 2. In some cases, a suitable type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Moraxella bovoculi AAX08_00205 Cas12a protein amino acid sequence depicted in FIG. 2. In some cases, a suitable type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Moraxella bovoculi AAX11_00205 Cas12a protein amino acid sequence depicted in FIG. 2. In some cases, a suitable type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Thiomicrospira sp.XSS Cas12a protein amino acid sequence depicted in FIG. 2. In some cases, a suitable type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Butyrivibrio sp. NC3005 Cas12a protein amino acid sequence depicted in FIG. 2. In some cases, a suitable type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the AACCas12b amino acid sequence depicted in FIG. 2.

In some cases, a type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with a Cas12b protein (e.g., a Cas protein depicted in FIG. 2). For example, in some cases, a type V CRISPR/Cas effector protein comprises an amino acid sequence having 50% or more sequence identity (e.g., 60% or more, 70% or more, 80% or more, 85% or more, 90% or more, 95% or more, 97% or more, 98% or more, 99% or more, or 100% sequence identity) with a Cas12b protein (e.g., a Cas12b protein depicted in FIG. 2). In some cases, a type V CRISPR/Cas effector protein comprises an amino acid sequence having 80% or more sequence identity (e.g., 85% or more, 90% or more, 95% or more, 97% or more, 98% or more, 99% or more, or 100% sequence identity) with a Cas12b protein (e.g., a Cas12b protein depicted in FIG. 2). In some cases, a type V CRISPR/Cas effector protein comprises an amino acid sequence having 90% or more sequence identity (e.g., 95% or more, 97% or more, 98% or more, 99% or more, or 100% sequence identity) with a Cas12b protein (e.g., a Cas12b protein depicted in FIG. 2). In some cases, a type V CRISPR/Cas effector protein comprises a Cas12b amino acid sequence depicted in FIG. 2.

In some cases, a suitable type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12b amino acid sequence depicted in FIG. 2A.

In some cases, a suitable type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12b amino acid sequence depicted in FIG. 2B.

In some cases, a suitable type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12b amino acid sequence depicted in FIG. 2C.

In some cases, a suitable type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12b amino acid sequence depicted in FIG. 2D.

In some cases, a suitable type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12b amino acid sequence depicted in FIG. 2E.

In some cases, a suitable type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12b amino acid sequence depicted in FIG. 2F.

In some cases, a suitable type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12b amino acid sequence depicted in FIG. 2G.

In some cases, a suitable type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12b amino acid sequence depicted in FIG. 2H.

In some cases, a suitable type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12b amino acid sequence depicted in FIG. 2I.

In some cases, a suitable type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12b amino acid sequence depicted in FIG. 2J.

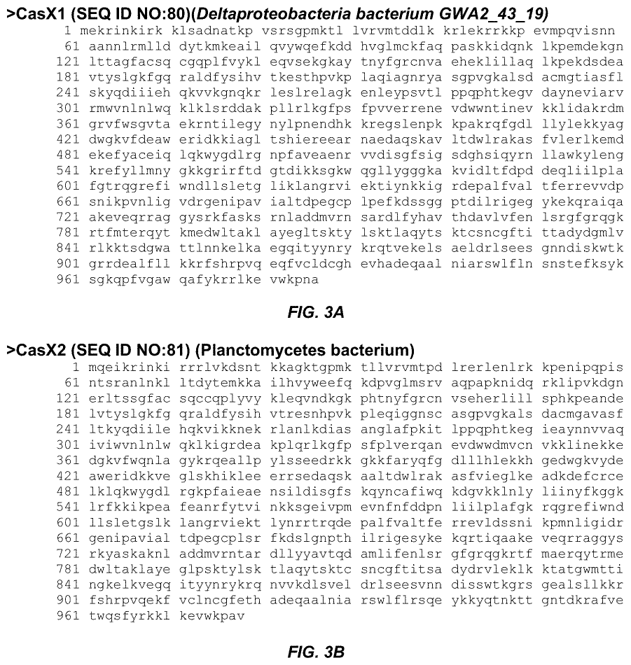

In some cases, a suitable type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12e amino acid sequence depicted in FIG. 3A.

In some cases, a suitable type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with the Cas12e amino acid sequence depicted in FIG. 3B.

In some cases, a type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with a Cas12, C2c4, C2c8, C2c5, C2c10, or C2c9 protein. For example, in some cases, a type V CRISPR/Cas effector protein comprises an amino acid sequence having 50% or more sequence identity (e.g., 60% or more, 70% or more, 80% or more, 85% or more, 90% or more, 95% or more, 97% or more, 98% or more, 99% or more, or 100% sequence identity) with a Cas12, C2c4, C2c8, C2c5, C2c10, or C2c9 protein. In some cases, a type V CRISPR/Cas effector protein comprises an amino acid sequence having 80% or more sequence identity (e.g., 85% or more, 90% or more, 95% or more, 97% or more, 98% or more, 99% or more, or 100% sequence identity) with a Cas12, C2c4, C2c8, C2c5, C2c10, or C2c9 protein. In some cases, a type V CRISPR/Cas effector protein comprises an amino acid sequence having 90% or more sequence identity (e.g., 95% or more, 97% or more, 98% or more, 99% or more, or 100% sequence identity) with a Cas12, C2c4, C2c8, C2c5, C2c10, or C2c9 protein. In some cases, a type V CRISPR/Cas effector protein comprises a Cas12, C2c4, C2c8, C2c5, C2c10, or C2c9 amino acid sequence.

In some cases, a type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with a Cas12, C2c4, C2c8, or C2c5 protein. For example, In some cases, a type V CRISPR/Cas effector protein comprises an amino acid sequence having 50% or more sequence identity (e.g., 60% or more, 70% or more, 80% or more, 85% or more, 90% or more, 95% or more, 97% or more, 98% or more, 99% or more, or 100% sequence identity) with a Cas12, C2c4, C2c8, or C2c5 protein. In some cases, a type V CRISPR/Cas effector protein comprises an amino acid sequence having 80% or more sequence identity (e.g., 85% or more, 90% or more, 95% or more, 97% or more, 98% or more, 99% or more, or 100% sequence identity) with a Cas12, C2c4, C2c8, or C2c5 protein. In some cases, a type V CRISPR/Cas effector protein comprises an amino acid sequence having 90% or more sequence identity (e.g., 95% or more, 97% or more, 98% or more, 99% or more, or 100% sequence identity) with a Cas12, C2c4, C2c8, or C2c5protein. In some cases, a type V CRISPR/Cas effector protein comprises a Cas12, C2c4, C2c8, or C2c5 amino acid sequence.

In some cases, a type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with a C2c4, C2c8, or C2c5 protein. For example, in some cases, a type V CRISPR/Cas effector protein comprises an amino acid sequence having 50% or more sequence identity (e.g., 60% or more, 70% or more, 80% or more, 85% or more, 90% or more, 95% or more, 97% or more, 98% or more, 99% or more, or 100% sequence identity) with a C2c4, C2c8, or C2c5 protein. In some cases, a type V CRISPR/Cas effector protein comprises an amino acid sequence having 80% or more sequence identity (e.g., 85% or more, 90% or more, 95% or more, 97% or more, 98% or more, 99% or more, or 100% sequence identity) with a C2c4, C2c8, or C2c5 protein. In some cases, a type V CRISPR/Cas effector protein comprises an amino acid sequence having 90% or more sequence identity (e.g., 95% or more, 97% or more, 98% or more, 99% or more, or 100% sequence identity) with a C2c4, C2c8, or C2c5 protein. In some cases, a type V CRISPR/Cas effector protein comprises a C2c4, C2c8, or C2c5 amino acid sequence.

In some cases, a type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with a Cas12, C2c10, or C2c9 protein. For example, In some cases, a type V CRISPR/Cas effector protein comprises an amino acid sequence having 50% or more sequence identity (e.g., 60% or more, 70% or more, 80% or more, 85% or more, 90% or more, 95% or more, 97% or more, 98% or more, 99% or more, or 100% sequence identity) with a Cas12, C2c10, or C2c9 protein. In some cases, a type V CRISPR/Cas effector protein comprises an amino acid sequence having 80% or more sequence identity (e.g., 85% or more, 90% or more, 95% or more, 97% or more, 98% or more, 99% or more, or 100% sequence identity) with a Cas12, C2c10, or C2c9 protein. In some cases, a type V CRISPR/Cas effector protein comprises an amino acid sequence having 90% or more sequence identity (e.g., 95% or more, 97% or more, 98% or more, 99% or more, or 100% sequence identity) with a Cas12, C2c10, or C2c9 protein. In some cases, a type V CRISPR/Cas effector protein comprises a Cas12, C2c10, or C2c9 amino acid sequence.

In some cases, a type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with a C2c10 or C2c9 protein. For example, In some cases, a type V CRISPR/Cas effector protein comprises an amino acid sequence having 50% or more sequence identity (e.g., 60% or more, 70% or more, 80% or more, 85% or more, 90% or more, 95% or more, 97% or more, 98% or more, 99% or more, or 100% sequence identity) with a C2c10 or C2c9 protein. In some cases, a type V CRISPR/Cas effector protein comprises an amino acid sequence having 80% or more sequence identity (e.g., 85% or more, 90% or more, 95% or more, 97% or more, 98% or more, 99% or more, or 100% sequence identity) with a C2c10 or C2c9 protein. In some cases, a type V CRISPR/Cas effector protein comprises an amino acid sequence having 90% or more sequence identity (e.g., 95% or more, 97% or more, 98% or more, 99% or more, or 100% sequence identity) with a C2c10 or C2c9 protein. In some cases, a type V CRISPR/Cas effector protein comprises a C2c10 or C2c9 amino acid sequence.

In some cases, a subject type V CRISPR/Cas effector protein (e.g., a Cas12 protein such as Cas12a, Cas12b, Cas12c, Cas12d, Cas12e) is fused to (conjugated to) a heterologous polypeptide. In some cases, a heterologous polypeptide (a fusion partner) provides for subcellular localization, i.e., the heterologous polypeptide contains a subcellular localization sequence (e.g., a nuclear localization signal (NLS) for targeting to the nucleus, a sequence to keep the fusion protein out of the nucleus, e.g., a nuclear export sequence (NES), a sequence to keep the fusion protein retained in the cytoplasm, a mitochondrial localization signal for targeting to the mitochondria, a chloroplast localization signal for targeting to a chloroplast, an endoplasmic reticulum (ER) retention signal, and the like). In some cases, a type V CRISPR/Cas effector protein (e.g., a Cas12 protein) does not include a NLS so that the protein is not targeted to the nucleus (which can be advantageous, e.g., when it desirable to cleave non-target ssDNAs in the cytosol). In some cases, the heterologous polypeptide can provide a tag (i.e., the heterologous polypeptide is a detectable label) for ease of tracking and/or purification (e.g., a fluorescent protein, e.g., a green fluorescent protein (GFP), a yellow fluorescent protein (YFP), a red fluorescent protein (RFP), a cyan fluorescent protein (CFP), mCherry, tdTomato, and the like; a histidine tag, e.g., a 6× His tag; a hemagglutinin (HA) tag; a FLAG tag; a Myc tag; and the like).

In some cases, a type V CRISPR/Cas effector protein (e.g., a Cas12 protein such as Cas12a, Cas12b, Cas12c, Cas12d, Cas12e) includes (is fused to) a nuclear localization signal (NLS) (e.g., in some cases 2 or more, 3 or more, 4 or more, or 5 or more NLSs). Thus, in some cases, a type V CRISPR/Cas effector protein includes one or more NLSs (e.g., 2 or more, 3 or more, 4 or more, or 5 or more NLSs). In some cases, one or more NLSs (2 or more, 3 or more, 4 or more, or 5 or more NLSs) are positioned at or near (e.g., within 50 amino acids of) the N-terminus and/or the C-terminus. In some cases, one or more NLSs (2 or more, 3 or more, 4 or more, or 5 or more NLSs) are positioned at or near (e.g., within 50 amino acids of) the N-terminus. In some cases, one or more NLSs (2 or more, 3 or more, 4 or more, or 5 or more NLSs) are positioned at or near (e.g., within 50 amino acids of) the C-terminus. In some cases, one or more NLSs (3 or more, 4 or more, or 5 or more NLSs) are positioned at or near (e.g., within 50 amino acids of) both the N-terminus and the C-terminus. In some cases, an NLS is positioned at the N-terminus and an NLS is positioned at the C-terminus.

In some cases, a type V CRISPR/Cas effector protein (e.g., a Cas12 protein such as Cas12a, Cas12b, Cas12c, Cas12d, Cas12e) includes (is fused to) between 1 and 10 NLSs (e.g., 1-9, 1-8, 1-7, 1-6, 1-5, 2-10, 2-9, 2-8, 2-7, 2-6, or 2-5 NLSs). In some cases, a type V CRISPR/Cas effector protein includes (is fused to) between 2 and 5 NLSs (e.g., 2-4, or 2-3 NLSs).

Non-limiting examples of NLSs include an NLS sequence derived from: the NLS of the SV40 virus large T-antigen, having the amino acid sequence PKKKRKV (SEQ ID NO:30); the NLS from nucleoplasmin (e.g., the nucleoplasmin bipartite NLS with the sequence KRPAATKKAGQAKKKK (SEQ ID NO:31)); the c-myc NLS having the amino acid sequence PAAKRVKLD (SEQ ID NO:32) or RQRRNELKRSP (SEQ ID NO:33); the hRNPA1 M9 NLS having the sequence NQSSNFGPMKGGNFGGRSSGPYGGGGQYFAKPRNQGGY (SEQ ID NO:34); the sequence RMRIZFKNKGKDTAELRRRRVEVSVELRKAKKDEQILKRRNV (SEQ ID NO:35) of the IBB domain from importin-alpha; the sequences VSRKRPRP (SEQ ID NO:36) and PPKKARED (SEQ ID NO:37) of the myoma T protein; the sequence PQPKKKPL (SEQ ID NO:38) of human p53; the sequence SALI AP (SEQ ID NO:39) of mouse c-abl IV; the sequences DRLRR (SEQ ID NO:40) and PKQKKRK (SEQ ID NO:41) of the influenza virus NS1; the sequence RKLKKKIKKL (SEQ ID NO:42) of the Hepatitis virus delta antigen; the sequence REKKKFLKRR (SEQ ID NO:43) of the mouse Mx1 protein; the sequence KRKGDEVDGVDEVAKKKSKK (SEQ ID NO:44) of the human poly(ADP-ribose) polymerase; and the sequence RKCLQAGMNLEARKTKK (SEQ ID NO:45) of the steroid hormone receptors (human) glucocorticoid. In general, NLS (or multiple NLSs) are of sufficient strength to drive accumulation of the protein in a detectable amount in the nucleus of a eukaryotic cell. Detection of accumulation in the nucleus may be performed by any suitable technique.

A Type V CRISPR/Cas effector protein binds to target DNA at a target sequence defined by the region of complementarity between the DNA-targeting RNA and the target DNA. As is the case for many CRISPR/Cas endonucleases, site-specific binding (and/or cleavage) of a double stranded target DNA occurs at locations determined by both (i) base-pairing complementarity between the guide RNA and the target DNA; and (ii) a short motif (referred to as the protospacer adjacent motif (PAM)) in the target DNA.

In some cases, the PAM for a Type V CRISPR/Cas effector protein is immediately 5′ of the target sequence (e.g., of the non-complementary strand of the target DNA—the complementary strand hybridizes to the guide sequence of the guide RNA while the non-complementary strand does not directly hybridize with the guide RNA and is the reverse complement of the non-complementary strand). In some cases (e.g., when Cas12a or Cas12b as described herein is used), the PAM sequence is 5′-TTN-3′. In some cases, the PAM sequence is 5′-TTTN-3.′ (e.g., see FIG. 2V).

In some cases, different Type V CRISPR/Cas effector proteins (i.e., Type V CRISPR/Cas effector proteins from various species) may be advantageous to use in the various provided methods in order to capitalize on a desired feature (e.g., specific enzymatic characteristics of different Type V CRISPR/Cas effector proteins). Type V CRISPR/Cas effector proteins from different species may require different PAM sequences in the target DNA. Thus, for a particular Type V CRISPR/Cas effector protein of choice, the PAM sequence requirement may be different than the 5′-TTN-3′ or 5′-TTTN-3′ sequence described above. Various methods (including in silico and/or wet lab methods) for identification of the appropriate PAM sequence are known in the art and are routine, and any convenient method can be used.

A nucleic acid molecule (e.g., a natural crRNA) that binds to a type V CRISPR/Cas effector protein (e.g., a Cas12 protein such as Cas12a, Cas12b, Cas12c, Cas12d, Cas12e), forming a ribonucleoprotein complex (RNP), and targets the complex to a specific target sequence within a target DNA is referred to herein as a “guide RNA.” It is to be understood that in some cases, a hybrid DNA/RNA can be made such that a guide RNA includes DNA bases in addition to RNA bases—but the term “guide RNA” is still used herein to encompass such hybrid molecules. A subject guide RNA includes a guide sequence (also referred to as a “spacer”) (that hybridizes to target sequence of a target DNA) and a constant region (e.g., a region that is adjacent to the guide sequence and binds to the type V CRISPR/Cas effector protein). A “constant region” can also be referred to herein as a “protein-binding segment.” In some cases, e.g., for Cas12a, the constant region is 5′ of the guide sequence.

The guide sequence has complementarity with (hybridizes to) a target sequence of the target DNA. In some cases, the guide sequence is 15-28 nucleotides (nt) in length (e.g., 15-26, 15-24, 15-22, 15-20, 15-18, 16-28, 16-26, 16-24, 16-22, 16-20, 16-18, 17-26, 17-24, 17-22, 17-20, 17-18, 18-26, 18-24, or 18-22 nt in length). In some cases, the guide sequence is 18-24 nucleotides (nt) in length. In some cases, the guide sequence is at least 15 nt long (e.g., at least 16, 18, 20, or 22 nt long). In some cases, the guide sequence is at least 17 nt long. In some cases, the guide sequence is at least 18 nt long. In some cases, the guide sequence is at least 20 nt long.

In some cases, the guide sequence has 80% or more (e.g., 85% or more, 90% or more, 95% or more, or 100% complementarity) with the target sequence of the target DNA. In some cases, the guide sequence is 100% complementary to the target sequence of the target DNA. In some cases, the target DNA includes at least 15 nucleotides (nt) of complementarity with the guide sequence of the guide RNA.

Examples of constant regions for guide RNAs that can be used with a type V CRISPR/Cas effector protein (e.g., a Cas12 protein such as Cas12a, Cas12b, Cas12c, Cas12d, Cas12e) are presented in FIG. 2.

In some cases, a subject guide RNA includes a nucleotide sequence having 70% or more identity (e.g., 80% or more, 85% or more, 90% or more, 95% or more, 98% or more, 99% or more, or 100% identity) with any one of the crRNA repeat sequences set forth in FIG. 2. In some cases, a subject guide RNA includes a nucleotide sequence having 90% or more identity (e.g., 95% or more, 98% or more, 99% or more, or 100% identity) with any one of the crRNA repeat sequences set forth in FIG. 2. In some cases, a subject guide RNA includes a crRNA nucleotide sequence set forth in FIG. 2.

In some cases, the guide RNA includes a double stranded RNA duplex (dsRNA duplex). In some cases, a guide RNA includes a dsRNA duplex with a length of from 2 to 12 bp (e.g., from 2 to 10 bp, 2 to 8 bp, 2 to 6 bp, 2 to 5 bp, 2 to 4 bp, 3 to 12 bp, 3 to 10 bp, 3 to 8 bp, 3 to 6 bp, 3 to 5 bp, 3 to 4 bp, 4 to 12 bp, 4 to 10 bp, 4 to 8 bp, 4 to 6 bp, or 4 to 5 bp). In some cases, a guide RNA includes a dsRNA duplex that is 2 or more bp in length (e.g., 3 or more, 4 or more, 5 or more, 6 or more, or 7 or more bp in length). In some cases, a guide RNA includes a dsRNA duplex that is longer than the dsRNA duplex of a corresponding wild type guide RNA. In some cases, a guide RNA includes a dsRNA duplex that is shorter than the dsRNA duplex of a corresponding wild type guide RNA.

In some cases, the constant region of a guide RNA is 15 or more nucleotides (nt) in length (e.g., 18 or more, 20 or more, 21 or more, 22 or more, 23 or more, 24 or more, 25 or more, 26 or more, 27 or more, 28 or more, 29 or more, 30 or more, 31 or more nt, 32 or more, 33 or more, 34 or more, or 35 or more nt in length). In some cases, the constant region of a guide RNA is 18 or more nt in length.

In some cases, the constant region of a guide RNA has a length in a range of from 12 to 100 nt (e.g., from 12 to 90, 12 to 80, 12 to 70, 12 to 60, 12 to 50, 12 to 40, 15 to 100, 15 to 90, 15 to 80, 15 to 70, 15 to 60, 15 to 50, 15 to 40, 20 to 100, 20 to 90, 20 to 80, 20 to 70, 20 to 60, 20 to 50, 20 to 40, 25 to 100, 25 to 90, 25 to 80, 25 to 70, 25 to 60, 25 to 50, 25 to 40, 28 to 100, 28 to 90, 28 to 80, 28 to 70, 28 to 60, 28 to 50, 28 to 40, 29 to 100, 29 to 90, 29 to 80, 29 to 70, 29 to 60, 29 to 50, or 29 to 40 nt). In some cases, the constant region of a guide RNA has a length in a range of from 28 to 100 nt. In some cases, the region of a guide RNA that is 5′ of the guide sequence has a length in a range of from 28 to 40 nt.

In some cases, the constant region of a guide RNA is truncated relative to (shorter than) the corresponding region of a corresponding wild type guide RNA. In some cases, the constant region of a guide RNA is extended relative to (longer than) the corresponding region of a corresponding wild type guide RNA. In some cases, a subject guide RNA is 30 or more nucleotides (nt) in length (e.g., 34 or more, 40 or more, 45 or more, 50 or more, 55 or more, 60 or more, 65 or more, 70 or more, or 80 or more nt in length). In some cases, the guide RNA is 35 or more nt in length.

As noted above, a method of the disclosure involves contacting the sample with a type V CRISPR/Cas effector protein and one or more guide RNAs, where the contacting step generates a PAM-distal cleavage product comprising a 5′ overhang; and ligating a double-stranded nucleic acid adapter to the cleavage product, to generate a ligation product. The double-stranded nucleic acid adapter comprises a 5′ overhang that comprises a stretch of from 3 to 15 contiguous nucleotides that are complementary to a contiguous stretch of nucleotides of the same length in the 5′ overhang of the PAM-distal cleavage product. The ligation product includes the target DNA, which can be sequenced.

An adapter nucleic acid includes any nucleic acid having a sequence, at least a portion of which is known, that can be joined to a target polynucleotide. Adapter nucleic acids can comprise DNA, RNA, nucleotide analogues, non-canonical nucleotides, labeled nucleotides, modified nucleotides, or combinations thereof. Adapter nucleic acids can be single-stranded, double-stranded, or partial duplex. In general, a partial-duplex adapter comprises one or more single-stranded regions and one or more double-stranded regions. A partial-duplex adapter can be described as a “double-stranded nucleic acid adapter comprising a 5′ overhang” (i.e., a 5′ single-stranded overhang).

An adapter nucleic acid for use in a method of the disclosure is double stranded, and comprises a 5′ overhang that comprises a stretch of from 3 to 15 contiguous nucleotides that are complementary to a contiguous stretch of nucleotides of the same length in the 5′ overhang of the PAM-distal cleavage product. In some cases, the 5′ overhang has a length of from 3 nucleotides to 20 nucleotides; for example, the 5′ overhang of the adapter nucleic acid can have a length of 3 nucleotides (nt), 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, or 20 nt. In some cases, the 5′ overhang has a length of from 8 nucleotides to 10 nucleotides. In some cases, the 5′ overhang has a length of 8 nucleotides. In some cases, the 5′ overhang has a length of 9 nucleotides. In some cases, the 5′ overhang has a length of 10 nucleotides. The 5′ overhang of the adapter nucleic acid can comprise a stretch of 3, 4, 5, 6, 7, 8, 9, 10, 11, 12, 13, 14, or 15 contiguous nucleotides that are complementary to a contiguous stretch of nucleotides of the same length in the 5′ overhang of the PAM-distal cleavage product. In some cases, the 5′ overhang of the adapter nucleic acid comprises a stretch of from 5 contiguous nucleotides to 10 contiguous nucleotides that are complementary to a contiguous stretch of nucleotides of the same length in the 5′ overhang of the PAM-distal cleavage product. In some cases, the 5′ overhang of the adapter nucleic acid comprises a stretch of from 8 contiguous nucleotides to 10 contiguous nucleotides that are complementary to a contiguous stretch of nucleotides of the same length in the 5′ overhang of the PAM-distal cleavage product. In some cases, the 5′ overhang of the adapter nucleic acid has a length of from 8 nucleotides to 10 nucleotides; and comprises a stretch of from 8 contiguous nucleotides to 10 contiguous nucleotides that are complementary to a contiguous stretch of nucleotides of the same length in the 5′ overhang of the PAM-distal cleavage product.

The total length of the adapter nucleic acid (including the 5′ overhang) can be from about 10 nucleotides to 100 nucleotides. For example, the total length of the adapter nucleic acid (including the 5′ overhang) can be from about 10 nucleotides (nt) to 15 nt, from 15 nt to 20 nt, from 20 nt to 25 nt, from 25 nt to 30 nt, from 30 nt to 40 nt, from 40 nt to 50 nt, from 50 nt to 60 nt, from 60 nt to 70 nt, from 70 nt to 80 nt, from 80 nt to 90 nt, or from 90 nt to 100 nt. In some cases, the total length of the adapter nucleic acid (including the 5′ overhang) is from about 10 nt to about 25 nt. In some cases, the total length of the adapter nucleic acid (including the 5′ overhang) is from about 15 nt to about 20 nt. In some cases, the total length of the adapter nucleic acid (including the 5′ overhang) is from about 15 nt to about 25 nt. In some cases, the total length of the adapter nucleic acid (including the 5′ overhang) is from about 15 nt to about 30 nt. In some cases, the total length of the adapter nucleic acid (including the 5′ overhang) is from about 25 nt to about 30 nt. In some cases, the total length of the adapter nucleic acid (including the 5′ overhang) is from about 20 nt to about 50 nt. In some cases, the total length of the adapter nucleic acid (including the 5′ overhang) is from about 25 nt to about 50 nt.

In some cases, the adapter nucleic acid comprises, in addition to the 5′ overhang discussed above, a 3′ overhang. In some cases, the adapter nucleic acid comprises, in addition to the 5′ overhang discussed above, a 3′ adenosine overhang.

The adapter nucleic acid is ligated to the PAM-distal cleavage product, to generate a ligation product comprising the adapter and the PAM-distal cleavage product. In some cases, the ligation product is further ligated to one or more additional adapters, e.g., an adapter that provides a bar code, an adapter that allows for next-generation sequencing, and the like. For example, the one or more additional adapters can include a nucleotide sequence specific for coupling to a sequencing platform; such an adapter may also include a barcode sequence. In some cases, the additional adapter comprises a nucleotide sequence that is at least 70% identical to a support-bound oligonucleotide conjugated to a solid support; in some cases, the solid support is coupled to a sequencing platform. In some cases, the additional adapter comprises a binding site for a sequencing primer. Ligation of various adapters to a type V CRISPR/Cas effector polypeptide cleavage product is depicted schematically in FIG. 1.

Double-stranded adapters can comprise two separate oligonucleotides hybridized to one another (also referred to as an “oligonucleotide duplex”), and hybridization may leave one or more blunt ends, one or more 3′ overhangs, one or more 5′ overhangs, one or more bulges resulting from mismatched and/or unpaired nucleotides, or any combination of these. In some embodiments, a single-stranded adapter comprises two or mores sequences that are able to hybridize with one another. When two such hybridizable sequences are contained in a single-stranded adapter, hybridization yields a hairpin structure (hairpin adapter). When two hybridized regions of an adapter are separated from one another by a non-hybridized region, a “bubble” structure results. Adapters comprising a bubble structure can consist of a single adapter oligonucleotide comprising internal hybridizations, or may comprise two or more adapter oligonucleotides hybridized to one another. Internal sequence hybridization, such as between two hybridizable sequences in an adapter, can produce a double-stranded structure in a single-stranded adapter oligonucleotide. Adapters of different kinds can be used in combination, such as a hairpin adapter and a double-stranded adapter, or adapters of different sequences. Hybridizable sequences in a hairpin adapter may or may not include one or both ends of the oligonucleotide. When neither of the ends are included in the hybridizable sequences, both ends are “free” or “overhanging.” When only one end is hybridizable to another sequence in the adapter, the other end forms an overhang, such as a 3′ overhang or a 5′ overhang. When both the 5′-terminal nucleotide and the 3′-terminal nucleotide are included in the hybridizable sequences, such that the 5′-terminal nucleotide and the 3′-terminal nucleotide are complementary and hybridize with one another, the end is referred to as “blunt.”

Adapters can contain one or more of a variety of sequence elements, including but not limited to, one or more amplification primer annealing sequences or complements thereof, one or more sequencing primer annealing sequences or complements thereof, one or more barcode sequences, one or more common sequences shared among multiple different adapters or subsets of different adapters, one or more restriction enzyme recognition sites, one or more overhangs complementary to one or more target polynucleotide overhangs, one or more probe binding sites (e.g. for attachment to a sequencing platform, such as a flow cell for massive parallel sequencing, such as developed by Illumina, Inc.), one or more random or near-random sequences (e.g. one or more nucleotides selected at random from a set of two or more different nucleotides at one or more positions, with each of the different nucleotides selected at one or more positions represented in a pool of adapters comprising the random sequence), and combinations thereof.

Two or more sequence elements can be non-adjacent to one another (e.g. separated by one or more nucleotides), adjacent to one another, partially overlapping, or completely overlapping. For example, an amplification primer annealing sequence can also serve as a sequencing primer annealing sequence. Sequence elements can be located at or near the 3′ end, at or near the 5′ end, or in the interior of the adapter oligonucleotide. When an adapter oligonucleotide is capable of forming secondary structure, such as a hairpin, sequence elements can be located partially or completely outside the secondary structure, partially or completely inside the secondary structure, or in between sequences participating in the secondary structure. For example, when an adapter oligonucleotide comprises a hairpin structure, sequence elements can be located partially or completely inside or outside the hybridizable sequences (the “stem”), including in the sequence between the hybridizable sequences (the “loop”). In some cases, the first adapter oligonucleotides in a plurality of first adapter oligonucleotides having different barcode sequences comprise a sequence element common among all first adapter oligonucleotides in the plurality. In some cases, all second adapter oligonucleotides comprise a sequence element common among all second adapter oligonucleotides that is different from the common sequence element shared by the first adapter oligonucleotides. A difference in sequence elements can be any such that least a portion of different adapters do not completely align, for example, due to changes in sequence length, deletion or insertion of one or more nucleotides, or a change in the nucleotide composition at one or more nucleotide positions (such as a base change or base modification). In some cases, an adapter oligonucleotide comprises a 5′ overhang, a 3′ overhang, or both that is complementary to one or more target polynucleotides. Complementary overhangs can be one or more nucleotides in length, including but not limited to 1, 2, 3, 4, 5, 6, 7, 8, 9,10, 11, 12, 13, 14, 15, or more nucleotides in length. Complementary overhangs may comprise a fixed sequence. Complementary overhangs may comprise a random sequence of one or more nucleotides, such that one or more nucleotides are selected at random from a set of two or more different nucleotides at one or more positions, with each of the different nucleotides selected at one or more positions represented in a pool of adapters with complementary overhangs comprising the random sequence. In some cases, an adapter overhang is complementary to a target polynucleotide overhang produced by restriction endonuclease digestion. In some embodiments, an adapter overhang consists of an adenine or a thymine.

In some cases, the nucleotide sequence is determined using next generation sequencing.

The term “next generation sequencing” (NGS) refers to the so-called highly parallelized methods of performing nucleic acid sequencing and comprises the sequencing-by-synthesis or sequencing-by-ligation platforms currently employed by Illumina, Life Technologies, Pacific Biosciences and Roche, etc. Next generation sequencing methods also include, but are not limited to, nanopore sequencing methods such as those offered by Oxford Nanopore, and electronic detection-based methods such as the Ion Torrent technology commercialized by Life Technologies.

As would be apparent to those skilled in the art, the ligation product may be amplified using primers that hybridize to the adapter present in the ligation product, thereby producing amplification products. In some cases, the primers used to amplify the fragments have a 5′ tail that provides compatibility with a particular sequencing platform. In certain cases, one or more of the primers used in this step may additionally contain a sample identifier (e.g., a bar code). If the primers have a sample identifier, then products from different samples can be pooled prior to sequencing. In some cases, this amplifying step may comprise appending a sample identifier sequence to the amplified fragments.

As would be apparent to those skilled in the art, the adapters and/or the primers used for amplification may be compatible with use in a next generation sequencing platform, e.g., Illumina's reversible terminator method, Roche's pyrosequencing method (454), Life Technologies' sequencing by ligation (the SOLiD platform), Life Technologies' Ion Torrent platform or Oxford Nanopore's MinIon system. Examples of such methods are described in the following references: Margulies et al. (Nature 2005 437: 376-80); Ronaghi et al. (Analytical Biochemistry 1996 242: 84-9); Shendure (Science 2005 309: 1728); Imelfort et al. (Brief Bioinform. 2009 10:609-18); Fox et al. (Methods Mol Biol. 2009; 553:79-108); Appleby et al. (Methods Mol Biol. 2009; 513:19-39) and Morozova (Genomics. 2008 92:255-64), which are incorporated by reference for the general descriptions of the methods and the particular steps of the methods, including all starting products, reagents, and final products for each of the steps. The present method may be used on any sequencing platform, including those that are based on sequencing-by-synthesis (i.e., by extending a primer that is hybridized to a template).

The DNA sequencing technology can utilize the Ion Torrent sequencing platform, which pairs semiconductor technology with a sequencing chemistry to directly translate chemically encoded information (A, C, G, T) into digital information (0, 1) on a semiconductor chip. Without wishing to be bound by theory, when a nucleotide is incorporated into a strand of DNA by a polymerase, a hydrogen ion is released as a byproduct. The Ion Torrent platform detects the release of the hydrogen atom as a change in pH. A detected change in pH can be used to indicate nucleotide incorporation. The Ion Torrent platform comprises a high-density array of micro-machined wells to perform this biochemical process in a massively parallel way. Each well holds a different library member, which may be clonally amplified. Beneath the wells is an ion-sensitive layer and beneath that an ion sensor. The platform sequentially floods the array with one nucleotide after another. When a nucleotide, for example a C, is added to a DNA template and is then incorporated into a strand of DNA, a hydrogen ion will be released. The charge from that ion will change the pH of the solution, which can be identified by Ion Torrent's ion sensor. If the nucleotide is not incorporated, no voltage change will be recorded and no base will be called. If there are two identical bases on the DNA strand, the voltage will be double, and the chip will record two identical bases called. Direct identification allows recordation of nucleotide incorporation in seconds. Library preparation for the Ion Torrent platform generally involves ligation of two distinct adaptors at both ends of a DNA fragment.

The DNA sequencing technology can utilize an Illumina sequencing platform, which generally employs cluster amplification of library members onto a flow cell and a sequencing-by-synthesis approach. Cluster-amplified library members are subjected to repeated cycles of polymerase-directed single base extension. Single-base extension can involve incorporation of reversible-terminator dNTPs, each dNTP labeled with a different removable fluorophore. The reversible-terminator dNTPs are generally 3′ modified to prevent further extension by the polymerase. After incorporation, the incorporated nucleotide can be identified by fluorescence imaging. Following fluorescence imaging, the fluorophore can be removed and the 3′ modification can be removed resulting in a 3′ hydroxyl group, thereby allowing another cycle of single base extension. Library preparation for the Illumina platform generally involves ligation of two distinct adaptors at both ends of a DNA fragment.

The DNA sequencing technology that is used can be the Helicos True Single Molecule Sequencing (tSMS), which can employ sequencing-by-synthesis technology. In the tSMS technique, a polyA adaptor can be ligated to the 3′ end of DNA fragments. The adapted fragments can be hybridized to poly-T oligonucleotides immobilized on the tSMS flow cell. The library members can be immobilized onto the flow cell at a density of about 10⁸ templates/cm². The flow cell can be then loaded into an instrument, e.g., HeliScope™ sequencer, and a laser can illuminate the surface of the flow cell, revealing the position of each template. A CCD camera can map the position of the templates on the flow cell surface. The library members can be subjected to repeated cycles of polymerase-directed single base extension. The sequencing reaction begins by introducing a DNA polymerase and a fluorescently labeled nucleotide. The polymerase can incorporate the labeled nucleotides to the primer in a template directed manner. The polymerase and unincorporated nucleotides can be removed. The templates that have directed incorporation of the fluorescently labeled nucleotide can be discerned by imaging the flow cell surface. After imaging, a cleavage step can remove the fluorescent label, and the process can be repeated with other fluorescently labeled nucleotides until a desired read length is achieved. Sequence information can be collected with each nucleotide addition step.

The DNA sequencing technology can utilize a SOLiD™ technology (Applied Biosystems). The SOLiD platform generally utilizes a sequencing-by-ligation approach. Library preparation for use with a SOLiD platform generally comprises ligation of adapters to the 5′ and 3′ ends of the DNA fragments (e.g., ligation products) to be sequenced to generate a fragment library. Alternatively, internal adapters can be introduced by ligating adapters to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations can be prepared in microreactors containing beads, primers, template, and PCR components. Following PCR, the templates can be denatured. Beads can be enriched for beads with extended templates. Templates on the selected beads can be subjected to a 3′ modification that permits bonding to a glass slide. The sequence can be determined by sequential hybridization and ligation of partially random oligonucleotides with a central determined base (or pair of bases) that is identified by a specific fluorophore. After a color is recorded, the ligated oligonucleotide can be removed and the process can then be repeated.

The DNA sequencing technology can utilize a single molecule, real-time (SMRT™) sequencing platform (Pacific Biosciences). In SMRT sequencing, the continuous incorporation of dye-labeled nucleotides can be imaged during DNA synthesis. Single DNA polymerase molecules can be attached to the bottom surface of individual zero-mode wavelength identifiers (ZMW identifiers) that obtain sequence information while phospholinked nucleotides are being incorporated into the growing primer strand. A ZMW generally refers to a confinement structure which enables observation of incorporation of a single nucleotide by DNA polymerase against a background of fluorescent nucleotides that rapidly diffuse in an out of the ZMW on a microsecond scale. By contrast, incorporation of a nucleotide generally occurs on a milliseconds timescale. During this time, the fluorescent label can be excited to produce a fluorescent signal, which is detected. Detection of the fluorescent signal can be used to generate sequence information. The fluorophore can then be removed, and the process repeated. Library preparation for the SMRT platform generally involves ligation of hairpin adaptors to the ends of DNA fragments.

The DNA sequencing technology can utilize nanopore sequencing (e.g. as described in Soni G V and Meller A. Clin Chem 53: 1996-2001 (2007)). Nanopore sequencing DNA analysis techniques are being industrially developed by a number of companies, including Oxford Nanopore Technologies (Oxford, United Kingdom). Nanopore sequencing is a single-molecule sequencing technology whereby a single molecule of DNA is sequenced directly as it passes through a nanopore. A nanopore can be a small hole, of the order of 1 nanometer in diameter. Immersion of a nanopore in a conducting fluid and application of a potential (voltage) across can result in a slight electrical current due to conduction of ions through the nanopore. The amount of current which flows is sensitive to the size and shape of the nanopore and to occlusion by, e.g., a DNA molecule. As a DNA molecule passes through a nanopore, each nucleotide on the DNA molecule obstructs the nanopore to a different degree, changing the magnitude of the current through the nanopore in different degrees. Thus, this change in the current as the DNA molecule passes through the nanopore represents a reading of the DNA sequence.

The DNA sequencing technology can utilize a chemical-sensitive field effect transistor (chemFET) array (e.g., as described in U.S. Patent Application Publication No. 20090026082). In one example of the technique, DNA molecules can be placed into reaction chambers, and the template molecules can be hybridized to a sequencing primer bound to a polymerase. Incorporation of one or more triphosphates into a new nucleic acid strand at the 3′ end of the sequencing primer can be discerned by a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

In some embodiments, a method of the disclosure comprises detecting a target DNA in a sample by use of a lateral flow assay based device. Lateral flow assay (LFA) based devices are among very rapidly growing strategies for qualitative and quantitative analysis. Lateral flow assays are performed over a strip, different parts of which are assembled on a plastic backing. These parts include a sample application pad, a conjugate pad, a nitrocellulose membrane and an adsorption pad. The nitrocellulose membrane is further divided into test and control lines. Pre-immobilized reagents at different parts of the strip become active upon flow of liquid sample. Lateral flow assays combine unique advantages of biorecognition probes and chromatography. Lateral flow assays basically combine a number of variants such as formats, biorecognition molecules, labels, detection systems and application.

Strips used for lateral flow assays contain four main components: a sample application pad, a conjugate pad, nitrocellulose membranes, and an adsorbent pad.

Sample application pad: The sample application pad is made of cellulose and/or glass fiber. The sample is applied on this pad to start the assay. Its function is to transport the sample to other components of lateral flow test strip (LFTS). The sample pad should be capable of transportation of the sample in a smooth, continuous and homogenous manner. The sample application pads are sometimes designed to pretreat the sample before its transportation. This pretreatment may include separation of sample components, removal of interfering agents, adjustment of pH, etc.

Conjugate pad: The conjugate pad is the place where labeled bio recognition molecules are dispensed. The material of conjugate pad should immediately release labeled conjugate upon contact with moving liquid sample. The labeled conjugate should stay stable over entire life span of the lateral flow strip. Any variations in dispensing, drying or release of conjugate can change results of the assay significantly. Poor preparation of labeled conjugate can adversely affect sensitivity of assay. Glass fiber, cellulose, poly-esters and some other materials are used to make conjugate pad for the lateral flow assay. The nature of the conjugate pad material has an effect on release of labeled conjugate and sensitivity of assay.

Nitrocellulose membrane: The Nitrocellulose membrane is highly important in determining sensitivity of the lateral flow assay. Nitrocellulose membranes are available in different grades. Test and control lines are drawn over this piece of membrane. An ideal membrane should provide support and good binding to capture probes (antibodies, aptamers etc.). Nonspecific adsorption over test and control lines may affect results of the assay significantly, thus a good membrane will be characterized by lesser non-specific adsorption in the regions of test and control lines. Wicking rate of nitrocellulose membrane can influence assay sensitivity. These membranes are easy to use, inexpensive, and offer high affinity for proteins and other biomolecules. Proper dispensing of bioreagents, drying and blocking play a role in improving sensitivity of assay.

Adsorbent pad: The adsorbent pad works as sink at the end of the strip. It also helps in maintaining the flow rate of the liquid over the membrane and stops back flow of the sample. The adsorbent capacity to hold liquid can play an important role in results of assay. All these components are fixed or mounted over a backing card.

Various formats can be adopted into the lateral flow assay, including the sandwich format, the competitive format and the multiplex detection format.

Sandwich Format: In a typical sandwich format, label (enzymes or nanoparticles or fluorescent dyes) coated antibody or aptamer is immobilized at the conjugate pad. This is a temporary adsorption which can be flushed away by flow of any buffer solution. A primary antibody or aptamer against target analyte is immobilized over test line. A secondary antibody or probe against labeled conjugate antibody/aptamer is immobilized at control zone. Sample containing the analyte is applied to the sample application pad and it subsequently migrates to the other parts of strip. At the conjugate pad, target analyte is captured by the immobilized labeled antibody or aptamer conjugate and results in the formation of labeled antibody conjugate/analyte complex. This complex now reaches the nitrocellulose membrane and moves under capillary action. At the test line, labeled antibody conjugate/analyte complex is captured by another antibody which is primary to the analyte. The analyte becomes sandwiched between the labeled and primary antibodies forming a labeled antibody conjugate/analyte/primary antibody complex. Excess labeled antibody conjugate will be captured at a control zone by a secondary antibody. Buffer or excess solution goes to absorption pad. The intensity of color at the test line corresponds to the amount of target analyte and is measured with an optical strip reader or visually inspected. Appearance of color at control line ensures that a strip is functioning properly.

Competitive format: A competitive format suits best for low molecular weight compounds which cannot bind two antibodies simultaneously. Absence of color at test line is an indication for the presence of analyte while appearance of color both at test and control lines indicates a negative result. The competitive format has two layouts. In the first layout, solution containing target analyte is applied onto the sample application pad and prefixed labeled biomolecule (antibody/aptamer) conjugate gets hydrated and starts flowing with the moving liquid. The test line contains pre-immobilized antigen (same analyte to be detected) which binds specifically to label conjugate. Control line contains pre-immobilized secondary antibody which has the ability to bind with labeled antibody conjugate. When liquid sample reaches at the test line, pre-immobilized antigen will bind to the labeled conjugate in case target analyte in sample solution is absent or present in such a low quantity that some sites of labeled antibody conjugate were vacant. Antigen in the sample solution and the one which is immobilized at test line of strip compete to bind with labeled conjugate. In another layout, labeled analyte conjugate is dispensed at conjugate pad while a primary antibody to analyte is dispensed at the test line. After application of analyte solution, a competition takes place between analyte and labeled analyte to bind with primary antibody at test line.

Multiplex detection: Multiplex detection format is used for detection of more than one target species and assay is performed over the strip containing test lines equal to number of target species to be analyzed. It is highly desirable to analyze multiple analytes simultaneously under same set of conditions. Multiplex detection format is very useful in clinical diagnosis where multiple analytes which are inter-dependent in deciding about the stage of a disease are to be detected. Lateral flow strips for this purpose can be built in various ways, i.e., by increasing length and test lines on conventional strip, making other structures like stars or T-shapes.

Various biorecognition molecules can be used with the lateral flow assay, including antibodies, aptamers, and molecular beacons.

Antibodies: Antibodies are employed as biorecognition molecules on the test and control lines of lateral flow strip and they bind to target analyte through immunochemical interactions. Resulting assay is known as lateral flow immunochromatographic assay (LFIA). Antibodies are available against common contaminants but they can also be synthesized against specific target analytes. An antibody which specifically binds to a certain target analyte is known as primary antibody but the one which is used to bind a target containing designs, formats and applications of lateral flow assay antibody or another antibody is known as secondary antibody.

Aptamers: Aptamers are the artificial nucleic acids and their discovery was reported by two groups in 1990. Aptamers have very high association constants and can bind selectively with a variety of target analytes. Organic molecules having molecular weights in the range of 100-10,000 Da are outstanding targets for aptamers. Because of their unique affinity toward target molecules, very closely related interferences can be differentiated. They are preferred over antibodies due to many features which include easy production process, simple labeling process, amplification after selection, straightforward structure modifications, unmatched stability, reproducibility and versatility of closely located quencher.

Molecular beacons: Molecular beacons can bind with high specificity and selectivity to nucleic acid sequences, toxins, proteins and other target molecules. Molecular beacons are composed of 15-30 base pairs in loop which are complimentary to target analyte and 4-6 base pairs at double stranded stem. Molecular beacons are being used in messenger RNA detection, intercellular imaging, protein and small molecule analysis, biosensors, biochip development, single nucleotide polymorphism and gene expression studies.

The list of materials that can be used as a label in a lateral flow assay is extensive and includes gold nanoparticles, colored latex beads, magnetic particles, carbon nanoparticles, selenium nanoparticles, silver nanoparticles, quantum dots, up converting phosphors, organic fluorophores, textile dyes, enzymes, liposomes and others. Any material that is used as a label should be detectable at very low concentrations and it should retain its properties upon conjugation with biorecognition molecules. This conjugation is also expected not to change the features of the bio-recognition probes. The ease in conjugation with biomolecules and stability over longer period of time are desirable features for a good label. Concentrations of labels down to 10-9M are optically detectable. After the completion of assay, some labels generate direct signals (as color from gold colloidal) while others require additional steps to produce analytical signals (as enzymes produce detectable product upon reaction with suit-able substrate). Hence the labels which give direct signal are preferable in LFA because of less time consumption and reduced procedure.

Colloidal gold nanoparticles are the most commonly used labels in LFA. Colloidal gold is inert and gives very perfect spherical particles. These particles have very high affinity toward biomolecules and can be easily functionalized. Optical properties of gold nanoparticles are dependent on size and shape. Size of particles can be tuned by use of suitable chemical additives. Their unique features include environment friendly preparation, high affinity toward proteins and biomolecules, enhanced stability, exceptionally higher values for charge transfer and good optical signaling. Optical properties of gold nanoparticle enhance sensitivity of analysis in LFA. Sensitivity is a function of molar absorption coefficient and accumulation of gold nanoparticles on target molecule. Optical signal of gold nanoparticles in colorimetric LFA can be amplified by deposition of silver, gold nanoparticles and enzymes.

Use of magnetic particles as colored labels in LFA has been reported by number of researchers. Colored magnetic particles produce color at the test line which is measured by an optical strip reader but magnetic signals coming from magnetic particles can also be used as detection signals and recorded by a magnetic assay reader. It has been reported that magnetic signals are stable for longer time compared to optical signals and they enhance sensitivity of LFA by 10 to 1000 folds

Fluorescent molecules are widely used in LFA as labels and the amount of fluorescence is used to quantitate the concentration of analyte in the sample. Detection of proteins is accomplished by using organic fluorophores such as rhodamine as labels in LFA. High photostability and brightness are required for LFAs.

Quantum dots are also used in LFAs. These semiconducting particles are not only water soluble but can also be easily combined with biomolecules because of closeness in dimensions. Owing to their unique optical properties, quantum dots have come up as a substitute to organic fluorescent dyes. Like gold nanoparticles QDs show size dependent optical properties and a broad spectrum of wavelengths can be monitored. Single light source is sufficient to excite quantum dots of all different sizes. QDs have high photostability and absorption coefficients. They can retain their fluorescent properties within the cells and bodies of organisms and less susceptible to metabolic degradation because of their inorganic nature.

Upconverting phosphors (UCP) are also labels which find use in LFAs. UPA labels are characterized by their excitation in infra-red region and emission in high energy visible region. Compared to other fluorescent materials, they have a unique advantage of not showing any auto fluorescence. Because of their excitation in IR regions, they do not photo degrade biomolecules. A major advantage lies in their production from easily available bulk materials. UCP particles were found to show size dependent sensitivity and specificity for detection of antibodies using LFA in sera of patients.

Enzymes are also employed as labels in LFA. But they increase one step in LFA which is application of suitable substrate after complete assay. This substrate will produce color at test and control lines as a result of enzymatic reaction. Horse-radish peroxidase labeled antibody conjugates can be used for detection of primary animal IgGs. In case of enzymes, selection of suitable enzyme substrate combination is one necessary requirement in order to get a colored product for strip reader or electroactive product for electrochemical detection. In other words, sensitivity of detection is dependent on the enzyme/substrate combination. Enhanced LFA sensitivity was observed when enzyme loaded gold nanoparticles were used as a label.

Colloidal carbon is comparatively inexpensive LFA label and its production can be easily scaled up. Because of their black color, carbon NPs can be easily detected with high sensitivity. Colloidal carbon can be functionalized with a large variety of biomolecules for detection of low and high molecular weight analytes. Carbon black nanoparticles showed very low detection limits compared to other labels. The sensitivity of LFA employing colloidal carbon is reported to be comparable with ELISA assay.

In case of gold nanoparticles or other color producing labels, qualitative or semi-quantitative analysis can be done by visual inspection of colors at test and control lines. The major advantage of visual inspection is rapid qualitative answer in “Yes” or “NO”. Such quick replies about presence of an analyte in clinical analysis have very high importance. Such tests can help doctors or other investigators to make an immediate decision, e.g., situations where test results from central labs cannot be waited for because of huge time consumption. But for quantification, optical strip readers are employed for measurement of the intensity of colors produced at test and control lines of strip. This is achieved by inserting the strips into a strip reader and intensities are recorded simultaneously by imaging software. Optical images of the strips can also be recorded with a camera and then processed by using a suitable software. Such systems use monochromatic light and wavelength of light can be adjusted to get a good contrast among test and control lines and background. Automated systems have advantages over manual imaging and processing in terms of time consumption, interpretation of results and adjustment of variables. In case of fluorescent labels, a fluorescence strip reader is used to record fluorescence intensity of test and control lines. Fluorescence brightness of a test line increases with an analyte's concentration in the sample. Magnetic strip readers and electrochemical detectors are also reported as detection systems in LFTS but they are not as common. Selection of detector is mainly determined by the label employed in analysis.

LFA strips give qualitative or semi-quantitative results which can be observed by naked eyes. Conventional LFAs are normally qualitative and give answers as a ‘yes’ or ‘no’ result. A good LFA biosensor should have the following properties: biocompatibility, high specificity, high sensitivity, rapidity of analysis, reproducibility/precision of results, wide working range of analysis, accuracy of analysis, high through-put, compactness, low cost, simplicity of operation, portability, flexibility in configuration, possibility of miniaturization, potential of mass production and on-site detection.

In some cases, a method of the disclosure comprises detecting a target DNA in a sample by amplifying target nucleic acids using one or more universal primers of Table 1, followed by use of CRISPR/Cas system. Type V CRISPR/Cas proteins, e.g., Cas12 proteins such as Cpf1 (Cas12a) and C2c1 (Cas12b) can promiscuously cleave non-targeted single stranded DNA (ssDNA) once activated by detection of a target DNA (double or single stranded). Once a type V CRISPR/Cas effector protein (e.g., a Cas12 protein such as Cas12a, Cas12b, Cas12c, Cas12d, Cas12e) is activated by a guide RNA, which occurs when the guide RNA hybridizes to a target sequence of a target DNA (i.e., the sample includes the targeted DNA), the protein becomes a nuclease that promiscuously cleaves ssDNAs (i.e., the nuclease cleaves non-target ssDNAs, i.e., ssDNAs to which the guide sequence of the guide RNA does not hybridize). Thus, when the target DNA is present in the sample (e.g., in some cases above a threshold amount), the result is cleavage of ssDNAs in the sample, which can be detected using any convenient detection method (e.g., using a labeled single stranded detector DNA). For example, in one embodiment, the sample is first contact with one or more universal primers of Table 1 to amplify nucleic acids of any pending target organisms in the sample, the amplified nucleic acids are then contacted with a Type V CRISPR/Cas system comprising a guide RNA that is specific for a sub-species of organism/nucleic acid in the amplified sample. In this way, specific identification of a sub-species of organisms can be identified in the population of DNA present in the sample.

The method includes, for example, amplifying DNA in a sample, using universal primers from Table 1 specific for bacterial species (e.g., one or more of SEQ ID Nos: 1-7 and/or 8). The amplified DNA will comprise all amplified 16S bacterial sequences in the sample. The amplified DNA is then contacted with a Type V CRISPR/Cas system in combination with guide RNA that are specific for one or more species of bacterial 16S nucleic acids in the sample to identify the presence of a particular species or sub-taxonomic category of bacteria. The particular “read out” from the Type V CRISPR/Cas method is further described below.

The method includes, for example, amplifying DNA in a sample, using universal primers from Table 1 specific for Babesia species (e.g., one or more of SEQ ID Nos: 9-14 and/or 15). The amplified DNA will comprise all amplified 18S Babesia sequences in the sample. The amplified DNA is then contacted with a Type V CRISPR/Cas system in combination with guide RNA that are specific for one or more species of Babesia 18S nucleic acids in the sample to identify the presence of a particular species or sub-taxonomic category of Babesia. The particular “read out” from the Type V CRISPR/Cas method is further described below.

The method includes, for example, amplifying DNA in a sample, using universal primers from Table 1 specific for Mycobacterium species (e.g., one or more of SEQ ID Nos: 16-22 and/or 23). The amplified DNA will comprise rpoB and/or hsp65 Mycobacterium sequences in the sample. The amplified DNA is then contacted with a Type V CRISPR/Cas system in combination with guide RNA that are specific for one or more species of Mycobacterium rpoB and/or hsp65 nucleic acids in the sample to identify the presence of a particular species or sub-taxonomic category of Mycobacterium. The particular “read out” from the Type V CRISPR/Cas method is further described below.

The method includes, for example, amplifying DNA in a sample, using universal primers from Table 1 specific for Fungal species (e.g., one or more of SEQ ID Nos: 24-28 and/or 29). The amplified DNA will comprise Fungal ribosomal sequences in the sample. The amplified DNA is then contacted with a Type V CRISPR/Cas system in combination with guide RNA that are specific for one or more species of fungal ribosomal nucleic acids in the sample to identify the presence of a particular species or sub-taxonomic category of fungus (fungi). The particular “read out” from the Type V CRISPR/Cas method is further described below.

Provided are compositions and methods for detecting a target DNA (double stranded or single stranded) in a sample. In some cases, a detector DNA is used that is single stranded (ssDNA) and does not hybridize with the guide sequence of the guide RNA (i.e., the detector ssDNA is a non-target ssDNA). Such methods can include (a) contacting the sample with: (i) a type V CRISPR/Cas effector protein (e.g., a Cas12 protein); (ii) a guide RNA comprising: a region that binds to the type V CRISPR/Cas effector protein, and a guide sequence that hybridizes with the target DNA; and (iii) a detector DNA that is single stranded and does not hybridize with the guide sequence of the guide RNA; and (b) measuring a detectable signal produced by cleavage of the single stranded detector DNA by the type V CRISPR/Cas effector protein, thereby detecting the target DNA. As noted above, once a subject Type V CRISPR/Cas effector protein (e.g., a Cas12 protein such as Cas12a, Cas12b, Cas12c, Cas12d, Cas12e) is activated by a guide RNA, which occurs when the sample includes a target DNA to which the guide RNA hybridizes (i.e., the sample includes the targeted target DNA), the Type V CRISPR/Cas effector protein (e.g., a Cas12 protein such as Cas12a, Cas12b, Cas12c, Cas12d, Cas12e) is activated and functions as an endoribonuclease that non-specifically cleaves ssDNAs (including non-target ssDNAs) present in the sample. Thus, when the targeted target DNA is present in the sample (e.g., in some cases above a threshold amount), the result is cleavage of ssDNA (including non-target ssDNA) in the sample, which can be detected using any convenient detection method (e.g., using a labeled detector ssDNA).

Also provided are compositions and methods for cleaving single stranded DNAs (ssDNAs) (e.g., non-target ssDNAs). Such methods can include contacting a population of nucleic acids, wherein said population comprises a target DNA and a plurality of non-target ssDNAs, with: (i) a type V CRISPR/Cas effector protein; and (ii) a guide RNA comprising: a region that binds to the type V CRISPR/Cas effector protein, and a guide sequence that hybridizes with the target DNA, wherein the type V CRISPR/Cas effector protein cleaves non-target ssDNAs of said plurality. Such a method can be used, e.g., to cleave foreign ssDNAs (e.g., viral DNAs) in a cell.

The guide RNA can be provided as RNA or as a nucleic acid encoding the guide RNA (e.g., a DNA such as a recombinant expression vector). The Type V CRISPR/Cas effector protein (e.g., a Cas12 protein such as Cas12a, Cas12b, Cas12c, Cas12d, Cas12e) can be provided as a protein or as a nucleic acid encoding the protein (e.g., an mRNA, a DNA such as a recombinant expression vector). In some cases, two or more (e.g., 3 or more, 4 or more, 5 or more, or 6 or more) guide RNAs can be provided by (e.g., using a precursor guide RNA array, which can be cleaved by the Type V CRISPR/Cas effector protein into individual (“mature”) guide RNAs).

In some cases (e.g., when contacting with a guide RNA and a Type V CRISPR/Cas effector protein (e.g., a Cas12 protein such as Cas12a, Cas12b, Cas12c, Cas12d, Cas12e)), the sample, after universal primer amplification, is contacted for 2 hours or less (e.g., 1.5 hours or less, 1 hour or less, 40 minutes or less, 30 minutes or less, 20 minutes or less, 10 minutes or less, or 5 minutes or less, or 1 minute or less) prior to the measuring step. For example, in some cases, the sample, after universal primer amplification, is contacted for 40 minutes or less prior to the measuring step. In some cases, the sample is contacted for 20 minutes or less prior to the measuring step. In some cases, the sample, after universal primer amplification, is contacted for 10 minutes or less prior to the measuring step. In some cases, the sample, after universal primer amplification, is contacted for 5 minutes or less prior to the measuring step. In some cases, the sample is contacted for 1 minute or less prior to the measuring step. In some cases, the sample, after universal primer amplification, is contacted for from 50 seconds to 60 seconds prior to the measuring step. In some cases, the sample, after universal primer amplification, is contacted for from 40 seconds to 50 seconds prior to the measuring step. In some cases, the sample, after universal primer amplification, is contacted for from 30 seconds to 40 seconds prior to the measuring step. In some cases, the sample, after universal primer amplification, is contacted for from 20 seconds to 30 seconds prior to the measuring step. In some cases, the sample, after universal primer amplification, is contacted for from 10 seconds to 20 seconds prior to the measuring step.

In some cases, the threshold of detection, for a subject method of detecting a target DNA in a sample, is 10 nM or less. The term “threshold of detection” is used herein to describe the minimal amount of target DNA that must be present in a sample in order for detection to occur. Thus, as an illustrative example, when a threshold of detection is 10 nM, then a signal can be detected when a target DNA is present in the sample at a concentration of 10 nM or more. In some cases, a method of the disclosure has a threshold of detection of 5 nM or less. In some cases, a method of the disclosure has a threshold of detection of 1 nM or less. In some cases, a method of the disclosure has a threshold of detection of 0.5 nM or less. In some cases, a method of the disclosure has a threshold of detection of 0.1 nM or less. In some cases, a method of the disclosure has a threshold of detection of 0.05 nM or less. In some cases, a method of the disclosure has a threshold of detection of 0.01 nM or less. In some cases, a method of the disclosure has a threshold of detection of 0.005 nM or less. In some cases, a method of the disclosure has a threshold of detection of 0.001 nM or less. In some cases, a method of the disclosure has a threshold of detection of 0.0005 nM or less. In some cases, a method of the disclosure has a threshold of detection of 0.0001 nM or less. In some cases, a method of the disclosure has a threshold of detection of 0.00005 nM or less. In some cases, a method of the disclosure has a threshold of detection of 0.00001 nM or less. In some cases, a method of the disclosure has a threshold of detection of 10 pM or less. In some cases, a method of the disclosure has a threshold of detection of 1 pM or less. In some cases, a method of the disclosure has a threshold of detection of 500 fM or less. In some cases, a method of the disclosure has a threshold of detection of 250 fM or less. In some cases, a method of the disclosure has a threshold of detection of 100 fM or less. In some cases, a method of the disclosure has a threshold of detection of 50 fM or less. In some cases, a method of the disclosure has a threshold of detection of 500 aM (attomolar) or less. In some cases, a method of the disclosure has a threshold of detection of 250 aM or less. In some cases, a method of the disclosure has a threshold of detection of 100 aM or less. In some cases, a method of the disclosure has a threshold of detection of 50 aM or less. In some cases, a method of the disclosure has a threshold of detection of 10 aM or less. In some cases, a method of the disclosure has a threshold of detection of 1 aM or less.

In some cases, the threshold of detection (for detecting the target DNA in a subject method), is in a range of from 500 fM to 1 nM (e.g., from 500 fM to 500 pM, from 500 fM to 200 pM, from 500 fM to 100 pM, from 500 fM to 10 pM, from 500 fM to 1 pM, from 800 fM to 1 nM, from 800 fM to 500 pM, from 800 fM to 200 pM, from 800 fM to 100 pM, from 800 fM to 10 pM, from 800 fM to 1 pM, from 1 pM to 1 nM, from 1 pM to 500 pM, from 1 pM to 200 pM, from 1 pM to 100 pM, or from 1 pM to 10 pM) (where the concentration refers to the threshold concentration of target DNA at which the target DNA can be detected). In some cases, a method of the disclosure has a threshold of detection in a range of from 800 fM to 100 pM. In some cases, a method of the disclosure has a threshold of detection in a range of from 1 pM to 10 pM. In some cases, a method of the disclosure has a threshold of detection in a range of from 10 fM to 500 fM, e.g., from 10 fM to 50 fM, from 50 fM to 100 fM, from 100 fM to 250 fM, or from 250 fM to 500 fM.

In some cases, the minimum concentration at which a target DNA can be detected in a sample is in a range of from 500 fM to 1 nM (e.g., from 500 fM to 500 pM, from 500 fM to 200 pM, from 500 fM to 100 pM, from 500 fM to 10 pM, from 500 fM to 1 pM, from 800 fM to 1 nM, from 800 fM to 500 pM, from 800 fM to 200 pM, from 800 fM to 100 pM, from 800 fM to 10 pM, from 800 fM to 1 pM, from 1 pM to 1 nM, from 1 pM to 500 pM, from 1 pM to 200 pM, from 1 pM to 100 pM, or from 1 pM to 10 pM). In some cases, the minimum concentration at which a target DNA can be detected in a sample is in a range of from 800 fM to 100 pM. In some cases, the minimum concentration at which a target DNA can be detected in a sample is in a range of from 1 pM to 10 pM.

In some cases, the threshold of detection (for detecting the target DNA in a subject method), is in a range of from 1 aM to 1 nM (e.g., from 1 aM to 500 pM, from 1 aM to 200 pM, from 1 aM to 100 pM, from 1 aM to 10 pM, from 1 aM to 1 pM, from 100 aM to 1 nM, from 100 aM to 500 pM, from 100 aM to 200 pM, from 100 aM to 100 pM, from 100 aM to 10 pM, from 100 aM to 1 pM, from 250 aM to 1 nM, from 250 aM to 500 pM, from 250 aM to 200 pM, from 250 aM to 100 pM, from 250 aM to 10 pM, from 250 aM to 1 pM, from 500 aM to 1 nM, from 500 aM to 500 pM, from 500 aM to 200 pM, from 500 aM to 100 pM, from 500 aM to 10 pM, from 500 aM to 1 pM, from 750 aM to 1 nM, from 750 aM to 500 pM, from 750 aM to 200 pM, from 750 aM to 100 pM, from 750 aM to 10 pM, from 750 aM to 1 pM, from 1 fM to 1 nM, from 1 fM to 500 pM, from 1 fM to 200 pM, from 1 fM to 100 pM, from 1 fM to 10 pM, from 1 fM to 1 pM, from 500 fM to 500 pM, from 500 fM to 200 pM, from 500 fM to 100 pM, from 500 fM to 10 pM, from 500 fM to 1 pM, from 800 fM to 1 nM, from 800 fM to 500 pM, from 800 fM to 200 pM, from 800 fM to 100 pM, from 800 fM to 10 pM, from 800 fM to 1 pM, from 1 pM to 1 nM, from 1 pM to 500 pM, from 1 pM to 200 pM, from 1 pM to 100 pM, or from 1 pM to 10 pM) (where the concentration refers to the threshold concentration of target DNA at which the target DNA can be detected). In some cases, a method of the disclosure has a threshold of detection in a range of from 1 aM to 800 aM. In some cases, a method of the disclosure has a threshold of detection in a range of from 50 aM to 1 pM. In some cases, a method of the disclosure has a threshold of detection in a range of from 50 aM to 500 fM.

In some cases, the minimum concentration at which a target DNA can be detected in a sample is in a range of from 1 aM to 1 nM (e.g., from 1 aM to 500 pM, from 1 aM to 200 pM, from 1 aM to 100 pM, from 1 aM to 10 pM, from 1 aM to 1 pM, from 100 aM to 1 nM, from 100 aM to 500 pM, from 100 aM to 200 pM, from 100 aM to 100 pM, from 100 aM to 10 pM, from 100 aM to 1 pM, from 250 aM to 1 nM, from 250 aM to 500 pM, from 250 aM to 200 pM, from 250 aM to 100 pM, from 250 aM to 10 pM, from 250 aM to 1 pM, from 500 aM to 1 nM, from 500 aM to 500 pM, from 500 aM to 200 pM, from 500 aM to 100 pM, from 500 aM to 10 pM, from 500 aM to 1 pM, from 750 aM to 1 nM, from 750 aM to 500 pM, from 750 aM to 200 pM, from 750 aM to 100 pM, from 750 aM to 10 pM, from 750 aM to 1 pM, from 1 fM to 1 nM, from 1 fM to 500 pM, from 1 fM to 200 pM, from 1 fM to 100 pM, from 1 fM to 10 pM, from 1 fM to 1 pM, from 500 fM to 500 pM, from 500 fM to 200 pM, from 500 fM to 100 pM, from 500 fM to 10 pM, from 500 fM to 1 pM, from 800 fM to 1 nM, from 800 fM to 500 pM, from 800 fM to 200 pM, from 800 fM to 100 pM, from 800 fM to 10 pM, from 800 fM to 1 pM, from 1 pM to 1 nM, from 1 pM to 500 pM, from 1 pM to 200 pM, from 1 pM to 100 pM, or from 1 pM to 10 pM). In some cases, the minimum concentration at which a target DNA can be detected in a sample is in a range of from 1 aM to 500 pM. In some cases, the minimum concentration at which a target DNA can be detected in a sample is in a range of from 100 aM to 500 pM.

In some cases, a subject composition or method exhibits an attomolar (aM) sensitivity of detection. In some cases, a subject composition or method exhibits a femtomolar (fM) sensitivity of detection. In some cases, a subject composition or method exhibits a picomolar (pM) sensitivity of detection. In some cases, a subject composition or method exhibits a nanomolar (nM) sensitivity of detection.

The disclosure provides a kit for carrying out a method of the disclosure (e.g., a method of characterizing a target DNA present in a sample).

In some cases, a kit of the disclosure comprises: A) one or more universal primers provided in Table 1; B) a type V CRISPR/Cas effector protein; C) one or more guide RNAs, where the one or more guide RNAs comprise: i) a region that binds to the type V CRISPR/Cas effector protein; and ii) a guide sequence that hybridizes with the target DNA; and C) a double-stranded nucleic acid adapter, where the adapter comprises a 5′ overhang that comprises a stretch of from 3 to 15 contiguous nucleotides that are complementary to a contiguous stretch of nucleotides of the same length in the 5′ overhang of a PAM-distal cleavage product generated by action of the type V CRISPR/Cas effector protein and the one or more guide RNAs on the target DNA.

In some cases, the kit also includes one or more reagents for determining the nucleotide sequence of a ligation product formed by ligating the adapter and the PAM-distal cleavage product.

In some cases, one or more components of a kit of the disclosure is lyophilized.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### EXAMPLES

RNA guides are designed to detect specific targets and are complexed with a CRISPR effector protein in the DETECTR assay. By pooling multiple RNA guides that targets different sequences, it is possible to detect multiple targets in the same reaction. It is also possible to detect a specific target with high sensitivity by pooling guides that targets different sequence segments of the same targets.

16S rRNA gene detection has been widely used in bacteria detection and identification. 16S rRNA genes from multiple species in a sample can be amplified with universal primer sets in the highly conserved region. The DETECTR assay targets the species- or genus-specific variable regions is then used to identify specific species or genius.

### Example 1

Guide Pooling for Detection of Borrelia Strains

The example here describes the screening of Borrelia specific guide RNAs.

Material and Reagents

RNA guides: 19 RNA guides have been designed to target 16S rRNA genes of Borrelia species, B. burgdorferi and/or B. miyamotoi.

Targets: Universal PCR Amplification Products and Double-Stranded Gene Fragment

Universal PCR amplification products: Amplicons were generated with a universal primer set (see, e.g., Table 1) (A35) of bacterial 16S rRNA genes from the following samples: (i) Borrelia culture diluted at various levels, 10⁻³ to 10⁻¹¹, in human plasma; (ii) Zymo standards: ZymoBIOMICS™ Microbial Community Standard containing Psuedomonas aeruginosa, E. coli, Salmonella enterica, Lactobacillus fermentum, Enterococcus faecalis, Staph aureus, Listeria monocytogenes, Bacillus subtilis, Saccharomyces cerevisiae, Cryptococcus neoformans; (iii) NOPS3: Negative control organisms including Rhodobacter sphaeroides, Aspergillus oryzae, Koi herpesvirus F347, Neospora caninum Nc-1, Streptococcus uberis 0140J, Pichia farinose; (iv) Other negative controls: negative plasma, no primer control, H2O.

Gene fragment: B. burgdorferi 16S fragment (“guide” 91 variant), 1607 bp.

Cas protein: Cas12M21

Reporter: The reporter is an 8-mer ssDNA with a FAM-labeled 5′ end and Iowa Black FQ-labeled 3′ end.

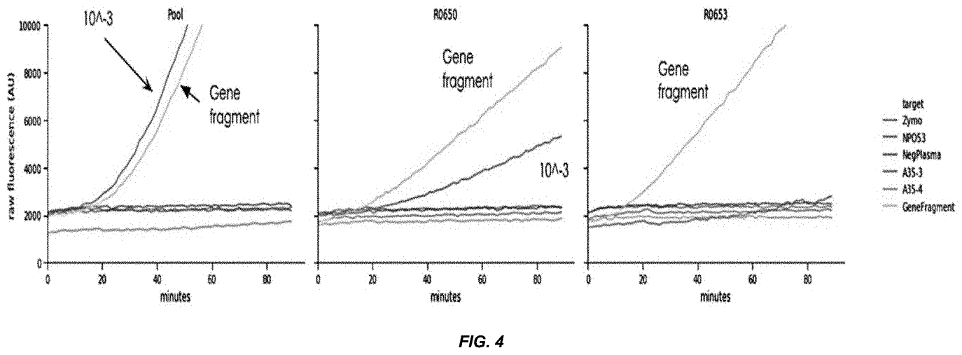

Guide pool and individual Borrelia guides that has high degree of homology (R0650) or no homology (R0653) to the conserved region of 16S rRNA genes in the Zymo standards were screened against the negative controls and diluted Borrelia culture samples. FIG. 4 shows the kinetic curve of the DETECTR reaction. There is no cross reactivity from the individual guides or guide pool to the Zymo controls or other negative controls (NPO3, negative plasma). The guides are active towards Borrelia target. Guide pooling increases the DETECTR signal, comparing to the individual guides.

### Example 2

Two sets of PCR Samples Using Different Universal Primer Sets (“D1” and “A35”) were Tested in the DETECTR Assay with Borrelia Guide Pool

In “D1” set, the Borrelia culture were diluted 10⁻³, 10⁻⁵, 10⁻⁷, in plasma. The negative control included negative plasma and H₂O.

In “A35” set, the Borrelia culture were diluted 10⁻³, 10⁻⁴, 10⁻⁵, 10⁻⁶, 10⁻⁷, 10⁻⁸, 10⁻⁹, 10⁻¹⁰, 10⁻¹¹ in plasma. The negative control included Zymo standards, NPO3, negative plasma, H2O, and no primer control.

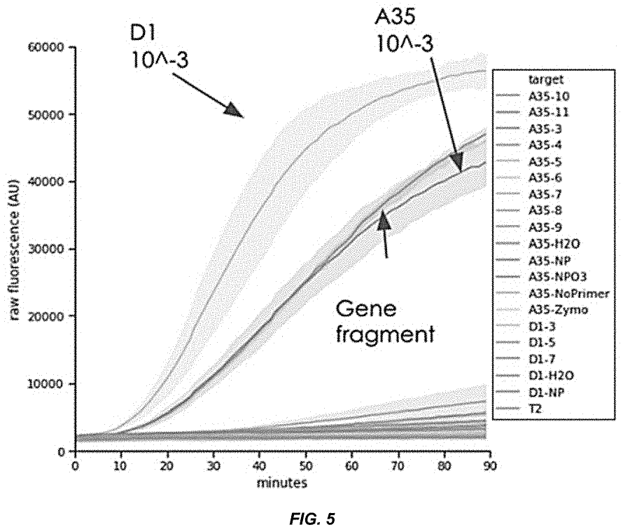

FIG. 5 is a graph that shows the kinetic curve of the DETECTR assay. In both D1 and A35 sets, 10⁻³ dilution samples are detected with the Borrelia guide pool. All negative control samples have no Borrelia signal in the DETECTR assay.

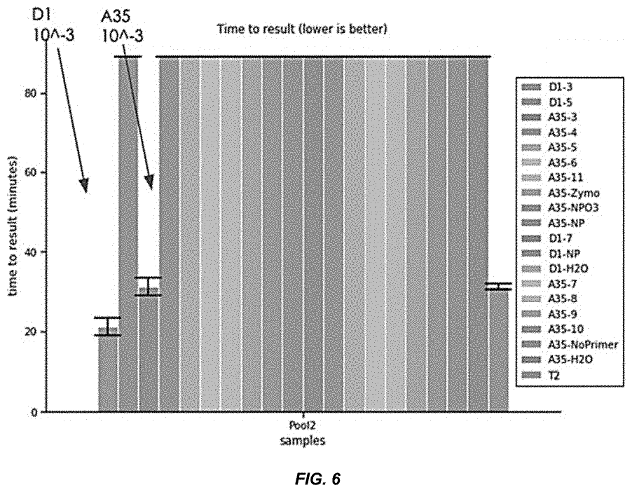

FIG. 6 shows the Time to results of the samples in the DETECTR assay. The threshold is dynamic set as 20% of the max fluorescence of the experiments, ˜12000 RFU. It takes ˜20 minutes to detect D1 10⁻³ dilution samples and ˜30 min to detect A35 10⁻³ dilution samples and 100 pM gene fragment.

Both experiments demonstrate the guide pool detect specific Borrelia signal without generating signal from co-amplified.

Example of high degree of homology of Borrelia guide to the conserved region of 16S rRNA genes (SEQ ID Nos: 46-51):

| | |
|---|---|
| Bacillus_subtilis_16S_1 | GACGGGGGCCCGCACAAGCGGTGGAGCATGTGGTTTAATTCGAAGCAACGCGAAGAACCT |
| Enterococcus_faecalis_16S_1 | GACGGGGGCCCGCACAAGCGGTGGAGCATGTGGTTTAATTCGAAGCAACGCGAAGAACCT |
| Escherichia_coli_16S_1 | GACGGGGGCCCGCACAAGCGGTGGAGCATGTGGTTTAATTCGATGCAACGCGAAGAACCT |
| Lactobacillus_fermentum_16S_1 | GACGGGGGCCCCCACAAGCGGTGGAGCATGTGGTTTAATTCGAAGCTACGCGAAGAACCT |
| Listersa_monocytogenes_16S_1 | GACGGGGGCCCGCACAAGCGGTGGAGCATGTGGTTTAATTCGAAGCAACGCGAAGAACCT |
| Pseudomnas_aeruginosa_16S_1 | GACGGGGGCCCGCACAAGCGGTGGAGCATGTGGTTTAATTCGAAGCAACCCGAAGAACCT |
| Salsonella_enterica_16S_1 | GACGGGGGCCCGCAGAAGCGGTGGAGCATGTGGTTTAATTCGATGCAACGCGAAGAACCT |
| Staphylococcus_aureus_16S_1 | GACGGGGACCCGCACAAGCGGTGGAGCATGTGGTTTAATTCGAAGCAACGCGAAGAACCT |
| burgdorferi fragment (reversed) | GACGGGGGCCCGCACAAGCGGTGGAGCATGTGGTTTAATTCGATGATACGCGAGGAACCT |
| B.miyamoti 16S | GACGGGGGCCCGCACAAGCGGTGGAGCATGTGGTTTAATTCGATGATACGCGAGGAACCT |
| guide 84 | -------------------------------------ATTCGATGATACGCGAGGAA--- |

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method for characterizing a target DNA present in a sample, the method comprising:
A) amplifying nucleic acids in the sample using at least one universal primer for a particular taxonomic rank of a desired organism to be detected to obtain amplified target DNA;
B) contacting the sample with:
a type V CRISPR/Cas effector protein; and
one or more guide RNAs, wherein the one or more guide RNAs comprise: i) a region that binds to the type V CRISPR/Cas effector protein; and ii) a guide sequence that hybridizes with the amplified target DNA,
a plurality of detector DNAs;
wherein said contacting generates a protospacer adjacent motif (PAM)-distal cleavage product comprising a 5′ overhang; and optionally
C) ligating a double-stranded nucleic acid adapter to the cleavage product, wherein the adapter comprises a 5′overhang that comprises a stretch of from 3 to 15 contiguous nucleotides that are complementary to a contiguous stretch of nucleotides of the same length in the 5′ overhang of the PAM-distal cleavage product, wherein said ligating generates a ligation product comprising the adapter and the PAM-distal cleavage product; and
determining the nucleotide sequence of the PAM-distal cleavage product present in the ligation product.

2. The method of claim 1, wherein the type V CRISPR/Cas effector protein is a Cas12 protein.

3. The method of claim 1, wherein the type V CRISPR/Cas effector protein is selected from the group consisting of a Cas12a (Cpf1) protein, a Cas12b (C2c1) protein, a Cas12d protein and a Cas14a protein.

4. (canceled)

5. (canceled)

6. (canceled)

7. The method of claim 1, wherein the amplified target DNA is single stranded.

8. The method of claim 1, wherein the amplified target DNA is double stranded.

9. The method of claim 1, wherein the amplified target DNA is selected from the group consisting of bacterial DNA, mycobacterium DNA, Babesia DNA, and fungal DNA.

10. (canceled)

11. (canceled)

12. (canceled)

13. The method of claim 1, wherein the at least one universal primer is selected from oligonucleotides having the sequence of SEQ ID NOs:1-7, or 8, or any two or more of SEQ ID NOs:1-8.

14. The method of claim 13, wherein the method is used to detect bacteria.

15. The method of claim 1, wherein the at least one universal primer is selected from oligonucleotides having the sequence of SEQ ID NOs:9-14, or 15, or any two or more of SEQ ID NOs:9-15.

16. The method of claim 15, wherein the method is used to detect babesia.

17. The method of claim 1, wherein the at least one universal primer is selected from oligonucleotides having the sequence of SEQ ID NOs:16-22, or 23, or any two or more of SEQ ID NOs:16-23.

18. The method of claim 17, wherein the method is used to detect mycobacteria.

19. The method of claim 1, wherein the at least one universal primer is selected from oligonucleotides having the sequence of SEQ ID NOs:24-28, or 29, or any two or more of SEQ ID NOs:24-29.

20. The method of claim 19, wherein the composition is used to detect fungi.

21. The method of claim 1, wherein the guide sequence of the guide RNA hybridizes to a sub-taxonomic classification of the particular taxonomic rank in the amplified target DNA.

22. The method of claim 1, wherein the method comprises contacting the sample with 2 or more guide RNAs, wherein the 2 or more guide RNAs differ from one another in the guide sequence.

23. The method of claim 22, comprising contacting the sample with 2 to 10 guide RNAs.

24. The method of claim 1, wherein the sample is a cell-free sample.

25. The method of claim 1, wherein the sample is blood, serum, plasma, bronchoalveolar lavage, sputum, urine, cerebrospinal fluid, feces, or a biopsy sample.

26. The method of claim 1, wherein said amplifying comprises isothermal amplification.

27. The method of claim 1, wherein said amplification comprises contacting the sample with 1 or more pairs of forward and reverse primers, wherein at least one primer is selected from primers having the sequence of SEQ ID NO:1-28 or 29.

28. The method of claim 1, wherein the adapter comprises a 3′ deoxyadenosine overhang.

29. The method of claim 1, wherein sequence determination is carried out by nanopore sequencing.

30. The method of claim 1, wherein the target DNA is present in the sample at a concentration as low as 200 fM.

31. The method of claim 1, wherein the detector DNA is single stranded and does not hybridize with the guide sequence of the guide RNA; and measuring a detectable signal produced by cleavage of the detector DNA by the type V CRISPR/Cas effector protein, thereby detecting the target DNA.

32. The method of claim 31, wherein the detector DNA comprises a fluorescence-emitting dye pair.

33. The method of claim 32, wherein the fluorescence-emitting dye pair is a fluorescence resonance energy transfer (FRET) pair.

34. The method of claim 33, wherein the fluorescence-emitting dye pair is a quencher/fluor pair.

35. The method of claim 1, wherein the detector DNA comprises a modified nucleobase, a modified sugar moiety, and/or a modified nucleic acid linkage.

36. A kit for characterizing a target DNA present in a sample, the system comprising:
A) one or more universal primers or primer pairs provided in SEQ ID NO: 1-28 and 29;
B) a type V CRISPR/Cas effector protein;
C) one or more guide RNAs, wherein the one or more guide RNAs comprise: i) a region that binds to the type V CRISPR/Cas effector protein; and ii) a guide sequence that hybridizes with the target DNA;
D) a plurality of detector DNAs; and optionally
E) a double-stranded nucleic acid adapter, wherein the adapter comprises a 5′overhang that comprises a stretch of from 3 to 15 contiguous nucleotides that are complementary to a contiguous stretch of nucleotides of the same length in the 5′ overhang of protospacer adjacent motif (PAM)-distal cleavage product generated by action of the type V CRISPR/Cas effector protein and the one or more guide RNAs on the target DNA.

37. The kit of claim 36, further comprising one or more reagents for determining the nucleotide sequence of a ligation product formed by ligating the adapter and the PAM-distal cleavage product.

38. The kit of claim 36, further comprising one or more reagents for amplifying the target DNA.
